(19) United States
(12) Patent Application Publication
(10) Pub. No.: US 2024/0285637 A1
(43) Pub. Date: Aug. 29, 2024

(54) PHOSPHODIESTERASE INHIBITORS FOR THE MITIGATION OF FRAGILE X SYNDROME SYMPTOMS

(71) Applicant: THE ROYAL INSTITUTION FOR THE ADVANCEMENT OF LEARNING/MCGILL UNIVERSITY, Montreal (CA)

(72) Inventor: Derek BOWIE, Verdun (CA)

(21) Appl. No.: 18/573,562

(22) PCT Filed: Jul. 8, 2022
(86) PCT No.: PCT/CA2022/000034

§ 371 Date: Dec. 22, 2023

Related U.S. Application Data
(60) Provisional application No. 63/219,524, filed on Jul. 8, 2021.

(51) Int. Cl. A61K 31/519; A61K 31/44; A61P 25/28

(52) U.S. Cl. CPC A61K 31/519; A61K 31/44; A61P 25/28

(57) ABSTRACT

The present disclosure concerns agents and therapeutic methods of mitigating at least one symptom of Fragile X syndrome (FXS) GRIN disorder, SynGAPI intellectual disability or Phelan-McDermid syndrome. The agents are inhibitors of one or more phosphodiesterase capable of hydrolyzing cGMP and optionally cAMP. In some embodiments, the agents can be inhibitors of a phosphodiesterase 1, 2, 5 or 10. The present disclosure also concerns a screening method for identifying test agents capable of mitigating at least one symptom of FXS. The screening methods determines that the test agent (or the combination of test agents) is useful when it is capable of increasing the activity of the neuronal nitric oxide synthase. Preferably, a mGluR5 blocking agent is combined with the phosphodiesterase inhibitors for the treatments described herein.

### CROSS-REFERENCE TO RELATED APPLICATIONS

This application claims priority from U.S. provisional application No. 63/219,524 filed Jul. 8, 2021 which is incorporated herein by reference in its entirety.

### TECHNOLOGICAL FIELD

The present invention pertains to agents that can mitigate one or more symptoms of Fragile X Syndrome as well as methods for identifying same.

### BACKGROUND

Fragile-X syndrome (FXS) is a neurodevelopmental disorder characterized by intellectual disabilities that range from mild to severe symptoms. Apart from intellectual impairment, individuals with FXS display typical physical features such as an elongated face, protruding ears and enlarged testes. They also tend to exhibit various behavioural, social and emotional challenges. Almost half of individuals with FXS have features associated with autism. In Canada, FXS affects 1/2500 to 1/4000 males and 1/2000 to 1/8000 females. Currently, there is no cure or specific treatment for this disorder.

Genetic studies have established that FXS results from elongation of the CGG trinucleotide repeat of the Fragile X messenger ribonucleoprotein 1 gene (Fmr1) located on the X-chromosome. The length of the CGG repeat determines the severity of the condition. In the most severe cases, the CGG repeat prevents any expression of Fragile X Messenger Ribonucleoprotein (FMRP), the gene product of Fmr1. FMRP is a RNA-binding protein that binds as many as 400 different brain mRNA transcripts and is essential for normal brain development.

FXS has been associated with a number of defects in the brain including deficits in signaling by glutamatergic and GABAergic neurotransmitters. Previous work has also established defects in serotonergic and muscarinic cholinergic transmission in FXS, as well as voltage-gated K-channels. One of the more promising hypothesis of FXS, the metabotropic glutamate receptor (mGluR) hypothesis, posits that the disease is caused by excessive local protein synthesis due to stimulation of the Group I mGluRs: mGluR1 and mGluR5. Indeed, many core features of FXS can be linked to exaggerated signaling by Group I mGluRs. However, despite promising preclinical findings, mGluR antagonists and GABAR agonists, have not shown clinical efficacy. This highlights the knowledge gap in our understanding the molecular basis of FXS.

It would be highly desirable to be provided with an effective therapeutic agent which can be used to mitigate one or more symptom of FXS. It would also be highly desirable to be provided with a screening method for identifying useful therapeutic agents to mitigate one or more symptom of FXS.

### BRIEF SUMMARY

The present disclosure concerns inhibitors of cGMP-hydrolyzing phosphodiesterases, and other agents, for the mitigation of one or more symptom of Fragile X syndrome (FXS).

According to a first aspect, the present disclosure provides a method of mitigating at least one symptom of FXS, in an individual in need thereof, that comprises administering to the individual a therapeutically effective amount of one or more inhibitors of the one or more phosphodiesterase capable of hydrolyzing cGMP. In some embodiments, the one or more phosphodiesterase comprises a cGMP-selective phosphodiesterase. In other embodiments, the one or more phosphodiesterase comprises phosphodiesterase 5 (PDE5). In still other embodiments, the PDE5 inhibitor comprises sildenafil or a pharmaceutically acceptable salt thereof. In additional embodiments, the one or more inhibitor of the one or more phosphodiesterase is selected from sildenafil, avanafil, tadalafil, vardenafil, udenafil, mirodenafil, iodenafil, zaprinast, icariin, and pharmaceutically acceptable salts thereof. In yet other embodiments, the one or more phosphodiesterase, that is capable of hydrolyzing cGMP, comprises a phosphodiesterase that is further capable of hydrolyzing cAMP. In other embodiments, the one or more phosphodiesterase comprises phosphodiesterase 1 (PDE1) 2 (PDE2) and/or 10 (PDE10). In further embodiments, the method further comprises administering a therapeutically effective amount of a mGluR5 blocking agent. In yet further embodiments, the mGluR5 blocking agent is an antagonist of mGluR5 or a negative allosteric modulator of mGluR5 and can be selected from the group consisting of 2-Methyl-6-(phenylethynyl)-pyridine (MPEP), methyl (3aR,4S,7aR)-4-hydroxy-4-[2-(3-methylphenyl)ethynyl]octahydro-1H-indole-1-carboxylate (mavoglurant), N-(3-Chlorophenyl)-N′-(1-methyl-4-oxo-4,5-dihydro-1H-imidazol-2-yl)urea (fenobam), 3-((2-Methyl-1,3-thiazol-4-yl)ethynyl)pyridine (MTEP), 6-methyl-2-(phenylazo)-3-pyridinol (SIB-1757), (E)-2-methyl-6-(2-phenylethenyl)pyridine (SIB-1893), basimglurant (2-chloro-4-{2-[1-(4-fluorophenyl)-2,5-dimethyl-1H-imidazol-4-yl]ethynyl}pyridine), 6-Fluoro-2-(4-(pyridin-2-yl)but-3-yn-1-yl)imidazo(1,2-a)pyridine (dipraglurant), 3-fluoro-5-[3-(5-fluoropyridin-2-yl)-1,2,4-oxadiazol-5-yl]benzonitrile (AZD 9272), 2-[(3-Fluorophenyl)ethynyl]-4,6-dimethyl-3-pyridinamine (raseglurant), N-(5-Fluoropyridin-2-yl)-6-methyl-4-(pyrimidin-5-yloxy)picolinamide (VU0424238), GRN-529 ([4-(Difluoromethoxy)-3-[2-(2-pyridinyl)ethynyl]phenyl](5,7-dihydro-6H-pyrrolo[3,4-b]pyridin-6-yl)-methanone), (6-Bromopyrazolo[1,5-a]pyrimidin-2-yl)[(1R)-1-methyl-3,4-dihydro-2(1H)-isoquinolinyl]methanone (remeglurant), (2S)-2-Amino-2-[(1S,2S)-2-carboxycycloprop-1-yl]-3-(xanth-9-yl)propanoic acid (LY-341495), GET73 (4-methoxy-N-[[4-(trifluoromethyl)phenyl]methyl]butanamide), arbaclofen ((3R)-4-amino-3-(4-chlorophenyl)butanoic acid), HTL-0014242 ((3-Chloro-5-[6-(5-fluoropyridin-2-yl)pyrimidin-4-yl]benzonitrile)), 2-chloro-N-[2-methoxy-4-(pyridin-2-yldiazenyl)phenyl]benzamide (Alloswitch1), PAM12,4-chloro-N-(6-(pyrimidin-5-yloxy)pyrazin-2-yl)picolinamide (VU-0431316), N-(4,4-dimethylcyclohexyl)pyrido[1′,2′:1,5]pyrazolo[4,3-d]pyrimidin-4-amine (VU-0467558), VU-0463841 (1-(5-chloropyridin-2-yl)-3-(3-cyano-5-fluorophenyl)urea), AP-612, LCGM-10, (3-fluorophenyl)[2-(5-fluoropyridin-2-yl)]-6,7-dihydoro[1,3]oxazolo[4,5-c]pyridin-5(4H)-yl]methanone (DSR-98776), EPX-105287, (αS)-α-Amino-α-[(1R,2R)-2-carboxycyclopropyl]-9H-xanthene-9-propanoic acid (LY-344545), MRZ-8676 (6,6-dimethyl-2-(2-phenylethynyl)-7,8-dihydroquinolin-5-one), 3-((4-(4-chlorophenyl)-7-fluoroquinolin-3-yl)sulfonyl)benzonitrile (RGH-618), 5-(3-chlorophenyl)-3-[(1R)-1-[(4-methyl-5-pyridin-4-yl-1,2,4-triazol-3-yl)oxy]ethyl]-1,2-oxazole (AZD-2066), AZD-2516, AZD-6538 (6-[5-(3-cyano-5-fluorophenyl)-1,2,4-oxadiazol-3-yl]pyridine-3-carbonitrile), and (RS)-α-methyl-4-carboxyphenylglycine ((RS)-MCPG). In still other embodiments, the method comprises administering an effective amount of at least two phosphodiesterase inhibitors to the individual. In some embodiments, when compared to at least one brain region in a control individual, the method is capable of facilitating in at least one brain region of the individual: a) intrinsic plasticity via a sodium channel; b) vasodilation; and/or c) GABAergic inhibitory synaptic plasticity. In some embodiments, the brain region is the cerebellum. In some embodiments, the individual is a human. In other embodiments, the individual is a child. In yet other embodiments, the individual is a baby. In some embodiments, the individual, has been diagnosed with FXS. In other embodiments, the at least one symptom of FXS comprises: a) hyperactivity; b) male aggression; c) anxiety; d) a learning deficit; e) a memory deficit; f) a sensory deficit; g) a sleep abnormality; and/or h) a repetitive behaviour.

According to a second aspect, the present disclosure provides the use of one or more inhibitor of the one or more phosphodiesterase capable of hydrolyzing cGMP for mitigating at least one symptom of FXS, in an individual in need thereof. The present disclosure also provides the use of one or more inhibitor of the one or more phosphodiesterase capable of hydrolyzing cGMP in the preparation of a medicament for mitigating at least one symptom of FXS, in an individual in need thereof. The present disclosure further comprises one or more inhibitor of the one or more phosphodiesterase capable of hydrolyzing cGMP for mitigating at least one symptom of FXS, in an individual in need thereof. In some embodiments, the one or more phosphodiesterase comprises a cGMP-selective phosphodiesterase. In other embodiments, the one or more phosphodiesterase comprises phosphodiesterase 5 (PDE5). In still other embodiments, the PDE5 inhibitor comprises sildenafil or a pharmaceutically acceptable salt thereof. In additional embodiments, the one or more inhibitor of the one or more phosphodiesterase is selected from sildenafil, avanafil, tadalafil, vardenafil, udenafil, mirodenafil, iodenafil, zaprinast, icarlin, and pharmaceutically acceptable salts thereof. In yet other embodiments, the one or more phosphodiesterase, that is capable of hydrolyzing cGMP, comprises a phosphodiesterase that is further capable of hydrolyzing cAMP. In other embodiments, the one or more phosphodiesterase comprises phosphodiesterases 1 (PDE1) 2 (PDE2) and/or 10 (PDE10). In still other embodiments at least two phosphodiesterase inhibitors are used. In further embodiments, the method further comprises administering a therapeutically effective amount of a mGluR5 blocking agent. In yet further embodiments, the mGluR5 blocking agent is an antagonist of mGluR5 or a negative allosteric modulator of mGluR5 and can be selected from the group consisting of 2-Methyl-6-(phenylethynyl)-pyridine (MPEP), methyl (3aR,4S,7aR)-4-hydroxy-4-[2-(3-methylphenyl)ethynyl]octahydro-1H-indole-1-carboxylate (mavoglurant), N-(3-Chlorophenyl)-N′-(1-methyl-4-oxo-4,5-dihydro-1H-imidazol-2-yl)urea (fenobam), 3-((2-Methyl-1,3-thiazol-4-yl)ethynyl)pyridine (MTEP), 6-methyl-2-(phenylazo)-3-pyridinol (SIB-1757), (E)-2-methyl-6-(2-phenylethenyl)pyridine (SIB-1893), basimglurant (2-chloro-4-{2-[1-(4-fluorophenyl)-2,5-dimethyl-1H-imidazol-4-yl]ethynyl}pyridine), 6-Fluoro-2-(4-(pyridin-2-yl)but-3-yn-1-yl)imidazo(1,2-a)pyridine (dipraglurant), 3-fluoro-5-[3-(S-fluoropyridin-2-yl)-1,2,4-oxadiazol-5-yl]benzonitrile (AZD 9272), 2-[(3-Fluorophenyl)ethynyl]-4,6-dimethyl-3-pyridinamine (raseglurant), N-(5-Fluoropyridin-2-yl)-6-methyl-4-(pyrimidin-5-yloxy)picolinamide (VU0424238), GRN-529 ([4-(Difluoromethoxy)-3-[2-(2-pyridinyl)ethynyl]phenyl](5,7-dihydro-6H-pyrrolo[3,4-b]pyridin-6-yl)-methanone), (6-Bromopyrazolo[1,5-a]pyrimidin-2-yl)[(1R)-1-methyl-3,4-dihydro-2(1H)-isoquinolinyl]methanone (remeglurant), (2S)-2-Amino-2-[(1S,2S)-2-carboxycycloprop-1-yl]-3-(xanth-9-yl)propanoic acid (LY-341495), GET73 (4-methoxy-N-[[4-(trifluoromethyl)phenyl]methyl]butanamide), arbaclofen ((3R)-4-amino-3-(4-chlorophenyl)butanoic acid), HTL-0014242 ((3-Chloro-5-[6-(5-fluoropyridin-2-yl)pyrimidin-4-yl]benzonitrile)), 2-chloro-N-[2-methoxy-4-(pyridin-2-yldiazenyl)phenyl]benzamide (Alloswitch1), PAM12,4-chloro-N-(6-(pyrimidin-5-yloxy)pyrazin-2-yl)picolinamide (VU-0431316), N-(4,4-dimethyloyclohexyl)pyrido[1′,2′:1,5]pyrazolo[4,3-d]pyrimidin-4-amine (VU-0467668), VU-0463841 (1-(5-chloropyridin-2-yl)-3-(3-cyano-5-fluorophenyl)urea), AP-612, LCGM-10, (3-fluorophenyl)[2-(5-fluoropyridin-2-yl)]-6,7-dihydoro[1,3]oxazolo[4,5-c]pyridin-5(4H)-yl]methanone (DSR-98776), EPX-105287, (αS)-α-Amino-α-[(1R,2R)-2-carboxycyclopropyl]-9H-xanthene-9-propanoic acid (LY-344545), MRZ-8676 (6,6-dimethyl-2-(2-phenylethynyl)-7,8-dihydroquinolin-5-one), 3-((4-(4-chlorophenyl)-7-fluoroquinolin-3-yl)sulfonyl)benzonitrile (RGH-618), 5-(3-chlorophenyl)-3-[(1R)-1-[(4-methyl-5-pyridin-4-yl-1,2,4-triazol-3-yl)oxy]ethyl]-1,2-oxazole (AZD-2066), AZD-2516, AZD-6538 (6-[5-(3-cyano-5-fluorophenyl)-1,2,4-oxadiazol-3-yl]pyridine-3-carbonitrile), and (RS)-α-methyl-4-carboxyphenylglycine ((RS)-MCPG). In some embodiments, when compared to at least one brain region in a control individual, the one or more phosphodiesterase inhibitors are capable of facilitating in at least one brain region of the individual: a) intrinsic plasticity via a sodium channel; b) vasodilation; and/or c) GABAergic inhibitory synaptic plasticity. In some embodiments, the brain region is the cerebellum. In some embodiments, the individual is a human. In other embodiments, the individual is a child. In yet other embodiments, the individual is a baby. In some embodiments, the individual, has been diagnosed with FXS. In other embodiments, the at least one symptom of FXS comprises: a) hyperactivity; b) male aggression; c) anxiety; d) a learning deficit; e) a memory deficit; f) a sensory deficit; g) a sleep abnormality; and/or h) a repetitive behaviour.

In a third aspect, the present disclosure provides a method of determining the usefulness of a test agent in the mitigation of a symptom of FXS. The method comprises contacting the test agent with a test cell capable of expressing neuronal nitric oxide synthase (nNOS), measuring a test level of activity of nNOS in the presence of the test agent, and determining that the test agent is useful if the test level of activity of nNOS is higher than a control level of activity obtained from a control cell. In some embodiments, the test agent is capable of inhibiting the activity of at least one phosphodiesterase capable of hydrolyzing cGMP. In other embodiments, the at least one phosphodiesterase comprises a selective cGMP phosphodiesterase. In further embodiments, the at least one phosphodiesterase comprises PDE 5. In other embodiments, the at least one phosphodiesterase, which is capable of hydrolyzing cGMP, is further capable of hydrolyzing cAMP. In yet other embodiments, the at least one phosphodiesterase comprises PDE1, PDE2 and/or PDE10. In some embodiments, the test cell is capable of expressing the N-methyl-D-aspartate receptor (NMDAR) and the method comprises measuring a test level of activity of the NMDAR in the presence of the test agent and determining that the test agent is useful if the level of activity of the NMDAR is lower than a control level of activity obtained from a control cell. In other embodiments, the method further comprises: a) contacting the test agent with a test brain sample comprising the test cell in order to obtain a treated brain sample; b) measuring, in the treated brain sample, one or more of the following to obtain test values: i) intrinsic plasticity via a sodium channel; ii) a degree of vasodilation; and/or iii) a level of GABAergic inhibitory synaptic plasticity; c) comparing the at least one test value obtained in (b) with the corresponding at least one control value obtained with a control brain sample comprising the control cell; and d) determining that the test agent is useful if the one or more test value is increased with respect to the one or more control value. Additionally, the test brain sample and the control brain sample are derived from an individual having FXS or an animal model of FXS. In some embodiments, the animal model is a mouse model. In further embodiments, the mouse model comprises a homozygous deletion of the Fmr1 gene. In some embodiments, the test brain sample and the control brain sample are derived from the same individual having FXS or the same animal model of FXS. In some embodiments, the method comprises measuring intrinsic plasticity via a sodium channel by determining the action current of cell-attached recordings. In other embodiments, the method comprises measuring the degree of vasodilation by determining the size and/or volume of cerebral blood vessels. In other embodiments, the method comprises measuring GABAergic inhibitory synaptic plasticity with current-clamp recordings. In yet other embodiments, the method comprises measuring GABAergic inhibitory synaptic plasticity with voltage-clamp recordings.

In a fourth aspect, the present disclosure provides a method of mitigating at least one symptom of Fragile X syndrome, GRIN disorder, SynGAP1 intellectual disability or Phelan-McDermid syndrome in an individual in need thereof, the method comprising administering a therapeutically effective amount of one or more inhibitor of one or more phosphodiesterase to the individual to mitigate the at least one symptom, wherein the one or more phosphodiesterase is capable of hydrolyzing cGMP. The present disclosure also provides the use of one or more inhibitor of the one or more phosphodiesterase capable of hydrolyzing cGMP in the preparation of a medicament for mitigating at least one symptom of FXS, in an individual in need thereof. The present disclosure further comprises one or more inhibitor of the one or more phosphodiesterase capable of hydrolyzing cGMP for mitigating at least one symptom of FXS, in an individual in need thereof. In some embodiments, the one or more phosphodiesterase comprises a cGMP-selective phosphodiesterase. In other embodiments, the one or more phosphodiesterase comprises phosphodiesterase 5 (PDE5). In still other embodiments, the PDE5 inhibitor comprises sildenafil or a pharmaceutically acceptable salt thereof. In additional embodiments, the one or more inhibitor of the one or more phosphodiesterase is selected from sildenafil, avanafil, tadalafil, vardenafil, udenafil, mirodenafil, iodenafil, zaprinast, icariin, and pharmaceutically acceptable salts thereof. In yet other embodiments, the one or more phosphodiesterase, that is capable of hydrolyzing cGMP, comprises a phosphodiesterase that is further capable of hydrolyzing cAMP. In other embodiments, the one or more phosphodiesterase comprises phosphodiesterases 1 (PDE1) 2 (PDE2) and/or 10 (PDE10). In still other embodiments at least two phosphodiesterase inhibitors are used. In further embodiments, the method further comprises administering a therapeutically effective amount of a mGluR5 blocking agent. In yet further embodiments, the mGluR5 blocking agent is an antagonist of mGluR5 or a negative allosteric modulator of mGluR5 and can be selected from the group consisting of 2-Methyl-6-(phenylethynyl)-pyridine (MPEP), methyl (3aR,4S,7aR)-4-hydroxy-4-[2-(3-methylphenyl)ethynyl]octahydro-1H-indole-1-carboxylate (mavoglurant), N-(3-Chlorophenyl)-N′-(1-methyl-4-oxo-4,5-dihydro-1H-imidazol-2-yl)urea (fenobam), 3-((2-Methyl-1,3-thiazol-4-yl)ethynyl)pyridine (MTEP), 6-methyl-2-(phenylazo)-3-pyridinol (SIB-1757), (E)-2-methyl-6-(2-phenylethenyl)pyridine (SIB-1893), basimglurant (2-chloro-4-[2-[1-(4-fluorophenyl)-2,5-dimethyl-1H-imidazol-4-yl]ethynyl)pyridine), 6-Fluoro-2-(4-(pyridin-2-yl)but-3-yn-1-yl)imidazo(1,2-a)pyridine (dipraglurant), 3-fluoro-5-[3-(5-fluoropyridin-2-yl)-1,2,4-oxadiazol-5-yl]benzonitrile (AZD 9272), 2-[(3-Fluorophenyl)ethynyl]-4,6-dimethyl-3-pyridinamine (raseglurant), N-(5-Fluoropyridin-2-yl)-6-methyl-4-(pyrimidin-5-yloxy)picolinamide (VU0424238), GRN-529 ([4-(Difluoromethoxy)-3-[2-(2-pyridinyl)ethynyl]phenyl](5,7-dihydro-6H-pyrrolo[3,4-b]pyridin-6-yl)-methanone), (6-Bromopyrazolo[1,5-a]pyrimidin-2-yl)[(1R)-1-methyl-3,4-dihydro-2(1H)-isoquinolinyl]methanone (remeglurant), (2S)-2-Amino-2-[(1 S,2S)-2-carboxycycloprop-1-yl]-3-(xanth-9-yl)propanoic acid (LY-341495), GET73 (4-methoxy-N-[[4-(trifluoromethyl)phenyl]methyl]butanamide), arbaclofen ((3R)-4-amino-3-(4-chlorophenyl)butanoic acid), HTL-0014242 ((3-Chloro-5-[6-(5-fluoropyridin-2-yl)pyrimidin-4-yl]benzonitrile)), 2-chloro-N-[2-methoxy-4-(pyridin-2-yldiazenyl)phenyl]benzamide (Alloswitch1), PAM12,4-chloro-N-(6-(pyrimidin-5-yloxy)pyrazin-2-yl)picolinamide (VU-0431316), N-(4,4-dimethylcyclohexyl)pyrido[1′,2′:1,5]pyrazolo[4,3-d]pyrimidin-4-amine (VU-0467558), VU-0463841 (1-(5-chloropyridin-2-yl)-3-(3-cyano-5-fluorophenyl)urea), AP-612, LCGM-10, (3-fluorophenyl)[2-(5-fluoropyridin-2-yl)]-6,7-dihydoro[1,3]oxazolo[4,5-c]pyridin-5(4H)-yl]methanone (DSR-98776), EPX-105287, (αS)-α-Amino-α-[(1R,2R)-2-carboxycyclopropyl]-9H-xanthene-9-propanoic acid (LY-344545), MRZ-8676 (6,6-dimethyl-2-(2-phenylethynyl)-7,8-dihydroquinolin-5-one), 3-((4-(4-chlorophenyl)-7-fluoroquinolin-3-yl)sulfonyl)benzonitrile (RGH-618), 5-(3-chlorophenyl)-3-[(1R)-1-[(4-methyl-5-pyridin-4-yl-1,2,4-triazol-3-yl)oxy]ethyl]-1,2-oxazole (AZD-2066), AZD-2516, AZD-6538 (6-[5-(3-cyano-5-fluorophenyl)-1,2,4-oxadiazol-3-yl]pyridine-3-carbonitrile), and (RS)-α-methyl-4-carboxyphenylglycine ((RS)-MCPG).

In a fifth aspect, there is provided the use of a therapeutically effective amount of one or more inhibitor of one or more phosphodiesterase to mitigate the symptoms of, treat, and/or prevent Fragile X syndrome, GRIN disorder, SynGAP1 intellectual disability or Phelan-McDermid syndrome in an individual in need thereof, and the one or more phosphodiesterase is capable of hydrolyzing cGMP. In some embodiments, the use further comprises administering a therapeutically effective amount of a mGluR5 blocking agent to the individual in need thereof. The therapeutically effective amount can be formulated as a salt or in a pharmaceutical composition. The one or more inhibitor of one or more phosphodiesterase and the mGluR5 blocking agent can be manufactured as a medicament. The present disclosure also provides the use of one or more inhibitor of the one or more phosphodiesterase capable of hydrolyzing cGMP in the preparation of a medicament for mitigating at least one symptom of FXS, in an individual in need thereof. The present disclosure further comprises one or more inhibitor of the one or more phosphodiesterase capable of hydrolyzing cGMP for mitigating at least one symptom of FXS, in an individual in need thereof. In some embodiments, the one or more phosphodiesterase comprises a cGMP-selective phosphodiesterase. In other embodiments, the one or more phosphodiesterase comprises phosphodiesterase 5 (PDE5). In still other embodiments, the PDE5 inhibitor comprises sildenafil or a pharmaceutically acceptable salt thereof. In additional embodiments, the one or more inhibitor of the one or more phosphodiesterase is selected from sildenafil, avanafil, tadalafil, vardenafil, udenafil, mirodenafil, iodenafil, zaprinast, icariin, and pharmaceutically acceptable salts thereof. In yet other embodiments, the one or more phosphodiesterase, that is capable of hydrolyzing cGMP, comprises—a phosphodiesterase that is further capable of hydrolyzing cAMP. In other embodiments, the one or more phosphodiesterase comprises phosphodiesterases 1 (PDE1) 2 (PDE2) and/or 10 (PDE10). In still other embodiments at least two phosphodiesterase inhibitors are used. In further embodiments, the method further comprises administering a therapeutically effective amount of a mGluR5 blocking agent. In yet further embodiments, the mGluR5 blocking agent is an antagonist of mGluR5 or a negative allosteric modulator of mGluR5 and can be selected from the group consisting of 2-Methyl-6-(phenylethynyl)-pyridine (MPEP), methyl (3aR,4S,7aR)-4-hydroxy-4-[2-(3-methylphenyl)ethynyl]octahydro-H-indole-1-carboxylate (mavoglurant), N-(3-Chlorophenyl)-N′-(1-methyl-4-oxo-4,5-dihydro-1H-imidazol-2-yl)urea (fenobam), 3-((2-Methyl-1,3-thiazol-4-yl)ethynyl)pyridine (MTEP), 6-methyl-2-(phenylazo)-3-pyridinol (SIB-1757), (E)-2-methyl-6-(2-phenylethenyl)pyridine (SIB-1893), basimglurant (2-chloro-4-{2-[1-(4-fluorophenyl)-2,5-dimethyl-1H-imidazol-4-yl]ethynyl}pyridine), 6-Fluoro-2-(4-(pyridin-2-yl)but-3-yn-1-yl)imidazo(1,2-a)pyridine (dipraglurant), 3-fluoro-5-[3-(5-fluoropyridin-2-yl)-1,2,4-oxadiazol-5-yl]benzonitrile (AZD 9272), 2-[(3-Fluorophenyl)ethynyl]-4,6-dimethyl-3-pyridinamine (raseglurant), N-(5-Fluoropyridin-2-yl)-6-methyl-4-(pyrimidin-5-yloxy)picolinamide (VU0424238), GRN-529 ([4-(Difluoromethoxy)-3-[2-(2-pyridinyl)ethynyl]phenyl](5,7-dihydro-6H-pyrrolo[3,4-b]pyridin-6-yl)-methanone), (6-Bromopyrazolo[1,5-a]pyrimidin-2-yl)[(1R)-1-methyl-3,4-dihydro-2(1H)-isoquinolinyl]methanone (remeglurant), (2S)-2-Amino-2-[(1S,2S)-2-carboxycycloprop-1-yl]-3-(xanth-9-yl)propanoic acid (LY-341495), GET73 (4-methoxy-N-[[4-(trifluromethyl)phenyl]methyl]butanamide), arbaclofen ((3R)-4-amino-3-(4-chlorophenyl)butanoic acid), HTL-0014242 ((3-Chloro-5-[6-(5-fluoropyridin-2-yl)pyrimidin-4-yl]benzonitrile)), 2-chloro-N-[2-methoxy-4-(pyridin-2-yldiazenyl)phenyl]benzamide (Alloswitch1), PAM12,4-chloro-N-(6-(pyrimidin-5-yloxy)pyrazin-2-yl)picolinamide (VU-0431316), N-(4,4-dimethylcyclohexyl)pyrido[1,2′:1,5]pyrazolo(4,3-d]pyrimidin-4-amine (VU-0467558), VU-0463841 (1-(5-chloropyridin-2-yl)-3-(3-cyano-5-fluorophenyl)urea), AP-612, LCGM-10, (3-fluorophenyl)[2-(5-fluoropyridin-2-yl)]-6,7-dihydoro[1,3]oxazolo[4,5-c]pyridin-5(4H)-yl]methanone (DSR-98776), EPX-105287, (αS)-α-Amino-α-[(1R,2R)-2-carboxycyclopropyl]-9H-xanthene-9-propanoic acid (LY-344545), MRZ-8676 (6,6-dimethyl-2-(2-phenylethynyl)-7,8-dihydroquinolin-5-one), 3-((4-(4-chlorophenyl)-7-fluoroquinolin-3-yl)sulfonyl)benzonitrile (RGH-618), 5-(3-chlorophenyl)-3-[(1R)-1-[(4-methyl-5-pyridin-4-yl-1,2,4-triazol-3-yl)oxy]ethyl]-1,2-oxazole (AZD-2066), AZD-2516, AZD-6538 (6-[5-(3-cyano-5-fluorophenyl)-1,2,4-oxadiazol-3-yl]pyridine-3-carbonitrile), and (RS)-α-methyl-4-carboxyphenylglycine ((RS)-MCPG).

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1. Schematic summarizing the signaling pathways triggered by N-methyl-D-aspartate receptors (NMDARs) expressed by molecular layer interneurons (stellate) (schematic adapted from Attwell, D., Buchan, A. M., Charpak, S., Lauritzen, M., MacVicar, B. A., & Newman, E. A. (2010). Glial and neuronal control of brain blood flow. Nature, 468(7321), 232-243.).

FIG. 2. Schematic of excitatory and inhibitory axons innervating cerebellar stellate cells and the positions of the stimulating and recording electrodes used for patch-clamping experiments.

FIG. 3A. Voltage-clamp records of stellate cells from wild-type mice following stimulation of parallel fibers (PFs) with a single stimulus sufficient to activate synaptic AMPAR responses.

FIG. 3B. Voltage-clamp records of stellate cells from Fmr1 KO mice (FXS model) following stimulation of PFs with a single stimulus sufficient to activate synaptic AMPAR responses.

FIG. 3C. Direct comparison of the amplitude and the decay kinetics of wild-type and Fragile-X AMPAR responses shown in FIG. 3A and FIG. 3B respectively. For the wild-type stellate cells, the Tfast was 1.88±0.3 and for the FXS stellate cells, the Tfast was 2.27±0.3.

FIG. 3D. Voltage-clamp records of stellate cells from wild-type (WT) mice that have been treated with 10 μM GYKI 53655 (to block AMPA currents) and subjected to either a single stimulus, to activate synaptic AMPAR responses, or a train of high frequency stimulation (HFS), to activate extrasynaptic NMDAR responses.

FIG. 3E. Voltage-clamp records of stellate cells from Fmr1 KO mice that have been treated with 10 μM GYKI 53655 (to block AMPA currents) and subjected to either a single stimulus, to activate synaptic AMPAR responses, or a train of high frequency stimulation (HFS), to activate extrasynaptic NMDAR responses.

FIG. 4A. Sample of action currents in cell-attached recordings from WT stellate cells in the presence of bicuculline collected at the beginning of the experiment (i.e. baseline).

FIG. 4B. Cell-attached recording from WT stellate cells during HFS of PFs which follows on from FIG. 4A.

FIG. 4C. Action currents in cell-attached recordings from WT stellate cells 25 minutes after HFS in FIG. 4B. Compared to data in FIG. 4A, the frequency of spontaneous action potentials has increased.

FIG. 4D. Action currents in cell-attached recordings from WT stellate cells pre-incubated with 10 μM (2R)-amino-5-phosphonovaleric acid (APV) (to block NMDA receptors) in the presence of bicuculline collected at the beginning of the experiment (i.e. baseline).

FIG. 4E. Cell-attached recordings from WT stellate cells pre-incubated with 10 μM APV during HFS of PFs which follows on from FIG. 4D.

FIG. 4F. Action currents in cell-attached recordings from WT stellate cells pre-incubated with 10 μM APV and 25 minutes after HFS (shown in FIG. 4E). Note that there is no increase in action potential firing revealing that the increase in excitability is due to the activation of NMDA receptors.

FIG. 4G. Action currents in cell-attached recordings from stellate cells of FMR1 KO mice in the presence of bicuculline collected at the beginning of the experiment (i.e. baseline).

FIG. 4H. Cell-attached recordings from stellate cells of FMR1 KO mice during HFS of PFs which follows on from FIG. 4G.

FIG. 4I. Action current in cell-attached recordings from FMR1 stellate cells 25 minutes after HFS (shown in FIG. 4H). Note that HFS of stellate cells lacking FMRP is unable to induce intrinsic plasticity.

FIG. 4J. Bar graph summarizing the firing rates from multiple WT stellate cells, in the presence of 10 μM bicuculline at baseline and 25 minutes after HFS of PFs. Data has been normalized to the baseline firing rates.

FIG. 4K. Bar graph summarizing the firing rates of WT stellate cells pre-incubated with 10 μM APV and in the presence of 10 μM bicuculline. Data are from baseline and 25 minutes after HFS of PFs. As before, data are normalized to the baseline firing rates in each condition.

FIG. 4L. Bar graph summarizing the firing rates of Fmr1 KO stellate cells pre-incubated with 10 μM APV and in the presence of 10 μM bicuculline. Data are from the baseline and 25 minutes after HFS of PFs. Data are normalized to the baseline firing rates in each condition.

FIG. 4M. Graph of the time course showing the effect of HFS on PF of stellate cells excitability for the three groups (WT, WT+APV, FMR1%). * indicates p≤0.05 and n.s. indicates p>0.05.

FIG. 4N. Summary bar graph showing the stellate cell basal firing rates of the three groups of FIG. 4M. * indicates p≤0.05 and n.s. indicates p>0.05.

FIG. 5A. Image of a capillary in the molecular layer of a cerebellar brain section of WT Fmr1 mice that were untreated (baseline).

FIG. 5B. Image of a capillary in the molecular layer of a cerebellar brain section of Fmr1 KO mice that were untreated (baseline).

FIG. 5C. Image of a capillary in the molecular layer of a cerebellar brain section of WT mice that were treated with 75 nM of the thromboxane A2 agonist U46619.

FIG. 5D. Image of a capillary in the molecular layer of a cerebellar brain section of Fmr1 KO mice that were treated with 75 nM of the thromboxane A2 agonist U46619.

FIG. 5E. Image of a capillary in the molecular layer of a cerebellar brain section of WT mice that were treated with 75 nM of U46616 followed by a 5-minute bath in 50 μM.

FIG. 5F. Image of a capillary in the molecular layer of a cerebellar brain section of Fmr1 KO mice that were treated with 75 nM of U46616 followed by a 5-minute bath in 50 μM NMDA.

FIG. 5G. The vasoconstriction of middle cerebral arteries (MCA) or posterior cerebral arteries (PCA) isolated from WT mice (n=9) and Fmr1 KO mice (n=4) in response to increasing concentrations of U44619, applied extraluminally, compared to baseline. * indicates p≤0.05.

FIG. 5H. Time course of vasodilation in different, individual capillaries from the molecular layer of the WT and Fmr1 KO mouse cerebellum. Pre-constriction is achieved by bath application of U44619 with subsequent bath application of 50 μM NMDA for 5 minutes. Single images of different time points of the experiment are shown in FIGS. 5A-5F. * indicates p≤0.05.

FIG. 5I. Graph showing the amount of dilation and constriction of cerebellar capillaries in brain sections from WT mice and Fmr1 KO mice. Slices were first treated with 75 nm U46619 to induce a contriction followed by 50 μM NMDA to promote dilation. Experiments were performed in the presence or absence of 100 μM sildenafil. * indicates p≤0.05.

FIG. 5J. Box plot showing a comparison of the resting diameter of the blood vessels measured in acutely isolated brains slices taken from the cerebellum and somatosensory cortex.

FIG. 5K. Graph showing a comparison of the vascular reactivity properties of blood vessels in the mouse cerebellum and cortex. Measurements of the degree of vasodilation observed in response to bath application of NMDA to blood vessels in the cerebellum and somatosensory cortex are shown. Note that the degree of vasodilation was similar in each case and that it was blocked by bath application of the neurotoxin, tetrodotoxin (TTX), demonstrating that NMDA-induced vasodilation is due to its actions on a neuron.

FIG. 5L. Graph showing a comparison of the degree of vasodilation induced by bath application of NMDA under different conditions to blood vessels in the cerebellum.

FIG. 5M. Graph showing a comparison of the degree of vasodilation induced by bath application of NMDA under different conditions to blood vessels in the cortex.

FIG. 5N. Graph showing a comparison of the degree of vasodilation observed in wildtype and Fmr1 KO blood vessels under different conditions in the cerebellum.

FIG. 5O. Graph showing a comparison of the degree of vasodilation observed in wildtype and Fmr1 KO blood vessels under different conditions in the cortex.

FIG. 6. Schematic of the nitric oxide/cGMP signaling pathway that promotes the recruitment of α3-GABARs into inhibitory synapses of cerebellar stellate cells.

FIG. 7A. Current clamp recordings of PF-evoked synaptic events in WT cerebellar brain slices, taken at the beginning of the experiment (i.e. baseline) and 25 mins after PF HFS.

FIG. 7B. Current clamp recordings of PF-evoked synaptic events in untreated Fmr1 KO cerebellar brain slices, taken at the beginning of the experiment (i.e. baseline) and 25 mins after PF HFS.

FIG. 7C. Current clamp recordings of PF-evoked synaptic events in Fmr1 KO cerebellar brain slices treated with 100 μM sildenafil, taken at the beginning of the experiment (i.e. baseline) and 25 mins after PF HFS.

FIG. 7D. Time course showing the effect of PF HFS on the dual excitatory/inhibitory postsynaptic potentials (EPSP) amplitudes in WT mice, untreated Fmr1 KO mice and Fmr1 KO mice treated with 100 μM sildenafil.

FIG. 7E. Summary bar graph showing peak EPSP amplitudes in WT and Fmr1 KO mice, in the presence or absence of 100 μM sildenafil.

FIG. 8A. HFS stimulation protocol and changes in membrane potential shown in FIG. 8B.

FIG. 8B. Voltage-clamp recordings of pharmacologically-isolated GABAA receptor-mediated membrane currents from cerebellar stellate cells of WT mice at baseline and 25 mins later in the recording (control).

FIG. 8C. Voltage-clamp recordings of pharmacologically-isolated GABAA receptor-mediated membrane currents from cerebellar stellate cells of WT mice that were subjected to HFS of PFs that was not paired with membrane depolarization (−60 mV HFS).

FIG. 8D. Voltage-clamp recordings of pharmacologically-isolated GABAA receptor-mediated membrane currents from cerebellar stellate cells of WT mice that were subject to HFS of PFs that was paired with depolarization (+40 mV HFS).

FIG. 8E. Voltage-clamp recordings of pharmacologically-isolated GABAA receptor-mediated membrane currents from cerebellar stellate cells of WT mice that were subject to HFS of PFs that was paired with depolarization (+40 mV HFS) in the presence of the calcium chelating agent 1,2-bis(o-aminophenoxy)ethane-N,N,N′,N′-tetraacetic acid) (BAPTA).

FIG. 8F. Voltage-clamp recordings of pharmacologically-isolated GABAA receptor mediated membrane currents from cerebellar stellate cells of Fmr1 KO mice subjected to HFS of PFs paired with depolarization (+40 mV HFS),

FIG. 8G. Voltage-clamp recordings of pharmacologically-isolated GABAA receptor mediated membrane currents from cerebellar stellate cells of Fmr1 KO mice treated with 100 μM sildenafil only.

FIG. 8H. Voltage clamp recordings of pharmacologically-isolated GABAA receptor mediated membrane currents from cerebellar stellate cells of Fmr1 KO mice subject to HFS of PFs paired with depolarization in the presence of 100 μM sildenafil.

FIG. 8I. Summary graph showing evoked peak inhibitory post-synaptic current (IPSC) amplitudes in WT and Fmr1 KO mice under different conditions.

FIG. 9A. Action currents recorded from WT stellate cells in the presence of 10 μM bicuculline.

FIG. 9B. Cell-attached recordings from WT stellate cells during HFS of PFs following on from FIG. 9A.

FIG. 9C. Action currents in cell-attached recordings from WT stellate cells 25 minutes after HFS (shown in FIG. 9B).

FIG. 9D. Action currents recorded from untreated WT stellate cell at baseline where GABAR inhibition is present.

FIG. 9E. Cell-attached recordings during HFS of PFs onto WT stellate cells where GABAR inhibition is present.

FIG. 9F. Action currents in cell-attached recordings from WT stellate cells 25 minutes after HFS (shown in FIG. 9F) when GABAR inhibition is present.

FIG. 9G. Action currents recorded from untreated Fmr1 KO stellate cells at baseline where GABAR inhibition is present.

FIG. 9H. Cell-attached recordings during HFS of PFs onto Fmr1 KO stellate cells where GABAR inhibition is present.

FIG. 9I. Action currents in cell-attached recordings from Fmr1 KO stellate cells 25 minutes after HFS (shown in FIG. 9I) when GABAR inhibition is present.

FIG. 9J. Action currents recorded from Fmr1 KO stellate cells in the presence of 100 μM sildenafil when GABAR inhibition is present.

FIG. 9K. Cell-attached recordings during HFS of PFs onto Fmr1 KO stellate cells (shown in FIG. 9K) where GABAR inhibition is present.

FIG. 9L. Action currents in cell-attached recordings from Fmr1 KO stellate cells 25 minutes after HFS (shown in FIG. 9L) when GABAR inhibition is present.

FIG. 9M. Time course showing the effect of PF HFS on WT stellate cell excitability (i.e. action current frequency) in the presence of 10 μM bicuculline.

FIG. 9N. Time course showing the effect of PF HFS on stellate cells excitability in WT mice when GABAR inhibition is present.

FIG. 9O. Time course showing the effect of PF HFS on stellate cells excitability in Fmr1 KO mice when GABAR inhibition is present.

FIG. 9P. Time course showing the effect of PF HFS on stellate cell excitability in Fmr1 KO mice, in the presence of 100 μM sildenafil when GABAR inhibition is present.

FIG. 10A. Schematic of the prepulse inhibition (PPI) behavioral assay set with mice.

FIG. 10B. Bar graph showing the deficits in prepulse inhibition (PPI) in Fmr1 KO mice rescued by administration of 7.5 mg/kg sildenafil. Results are shown, from left to right, for the WT mouse (in the absence or the presence of sildenafil) as well as the Fmr1^{-/-} mouse (in the absence of presence of sildenafil). * indicates p≤0.05.

FIG. 10C. Bar graph showing the amplitude of prepulse inhibition (PPI) in both WT and Fmr1 KO mice in the different conditions tested. Results are shown, from left to right, for the WT mouse (in the absence or the presence of sildenafil) as well as the Fmr1^{-/-} mouse (in the absence of presence of sildenafil). * indicates p≤0.05.

FIG. 10D. Schematic of the open field locomotion test.

FIG. 10E. Deficits in locomotion in Fmr1 KO mice were rescued by administration of 7.5 mg/kg sildenafil. Data are shown for the WT mouse (in the absence or the presence of sildenafil) as well as the Fmr1/mouse (in the absence of presence of sildenafil). * indicates p≤0.05.

FIG. 10F. Bar graph showing the total locomotion measured for the WT and Fmr1 KO mice shown in FIG. 10E. Results of the open field locomotion test are provided as locomotion time (sec) that the mouse spent moving (y-axis) as a function of the time or duration of the experiment (x-axis).

FIG. 11A. RNA-seq data showing the relative expression of PDE5a isoform transcripts in NOS1+ neurons compiled using the mousebrain.org online public database.

FIG. 11B. RNA-seq data showing the relative expression of PDE2a isoform transcripts in NOS1+ neurons compiled using the mousebrain.org online public database.

FIG. 11C, RNA-seq data showing the relative expression of PDE10a isoform transcripts in NOS1+ neurons compiled using the mousebrain.org online public database.

FIG. 11D. RNA-seq data showing the relative expression of PDE1a & PDE1b isoform transcripts in NOS1-neurons compiled using the mousebrain.org online public database.

FIG. 12A. Voltage-clamp recordings of pharmacologically-isolated GABA-A receptor mediated synaptic events that were evoked in cerebellar stellate cells using a minimal stimulation protocol.

FIG. 12B. Voltage-clamp recordings of pharmacologically-isolated GABA-A receptor mediated synaptic events that were evoked with minimal stimulation following high frequency stimulation (HFS) of parallel fibers.

FIG. 12C. Example of peak GABAR-evoked responses during a 5 minute period (cell #20200220p1) using the minimal stimulation protocol. Note that the stimulation protocol elicited both failures and synaptic events.

FIG. 12D. Example of peak GABAR-evoked responses (cell #20200220p1) observed after HFS. Note that there are fewer event failures and more evoked events which can be explained by the occurrence of silent GABAergic synapses.

FIG. 12E. Graph summarizing the failure rate (%) for each recording from WT STELLATEs at baseline and after HFS. Note that the number of event failures decreased in all cells after HFS. The mean value is indicated by a star.

FIG. 12F. Graph of the failure rate as percent of baseline of each WT cell plotted against the change in failure rate following HFS. The mean is denoted by a star which illustrates that the initial baseline failure rate did not impact the increase in synaptic connectivity. The mean value is indicated by a star.

FIG. 13A. Frequency histogram at the baseline before HFS (data from experiment shown in FIG. 12A). The graph illustrates the most commonly occurring events are under −100 pA. The graph was fit with three Gaussian functions.

FIG. 13B. Square root of the frequency histogram at the baseline before HFS (cells from FIG. 12A). The graph illustrates full range of amplitudes across all cells (up to −2000 pA).

FIG. 13C. Frequency histogram post-HFS (data from experiment shown in FIG. 12B), The graph illustrates the most commonly occurring events are under −100 pA under the baseline condition. The graph was fit with three Gaussian functions.

FIG. 13D. Square root of the frequency histogram post-HFS (data from experiment shown in FIG. 12B). The graph illustrates full range of amplitudes across all cells (up to −2000 pA).

FIG. 13E. Graph showing the decay kinetics from all synaptic events (from FIG. 13A) measured at baseline plotted against their amplitude. The graph emphasizes the most commonly occurring events up to −500 pA in amplitude.

FIG. 13F. Graph showing the decay kinetics from all synaptic events (from FIG. 13A) measured at baseline plotted against their amplitude. The graph illustrates the full range of amplitudes and decay kinetics recorded.

FIG. 13G. Graph showing the decay kinetics from all synaptic events (from FIG. 13B) measured post-HSF plotted against their amplitude. The graph emphasizes the most commonly occurring events up to −500 pA in amplitude,

FIG. 13H. Graph showing the decay kinetics from all synaptic events (from FIG. 13B) measured post-HSF plotted against their amplitude. The graph illustrates the full range of amplitudes and decay kinetics recorded.

FIG. 14A. Graph showing the time latency of evoked synaptic events occurred within 0.5 to 5 ms prior to HFS.

FIG. 14B. Graph showing the time latency of evoked synaptic events occurred within 0.5 to 5 ms after HFS.

FIG. 14C. Plot of the peak response amplitudes before HFS in WT mice.

FIG. 14D. Plot of the peak response amplitudes after HFS in WT mice. Note that there are fewer large amplitude events.

FIG. 15. Schematic illustrating the co-existence of inhibitory long-term potentiation (iLTP) and inhibitory synapse long-term depression (iLTD) at inhibitory synapses of WT cerebellar stellate cells.

FIG. 16A. Voltage-clamp recordings of a raw trace of a stellate cell from an α3 KO mouse (cell no. 20200820p1) at the baseline condition.

FIG. 16B. Voltage-clamp recordings of a raw trace of a stellate cell from an α3 KO mouse (cell no. 20200820p1) post-HSF.

FIG. 16C. Scatter plot illustrating the failure rate of GABAergic transmission (henceforth “the failure rate”) with an example of a cell (cell no. 20200904p1) at baseline from α3 KO mouse (from FIG. 16A).

FIG. 16D. Scatter plot illustrating the failure rate with an example of a cell (cell no. 20200904p1) post-HSF from α3 KO mouse (from FIG. 16B).

FIG. 16E. Summary graph depicting the raw failure rate percentages for all cells at baseline and post-HFS in α3 KO mice. The mean is represented by a star. The graph shows that all cells increased their failure rate post-HFS.

FIG. 16F. Graph showing the initial failure rate of all cells compared to how much that cell changed post-HFS in α3 KO mice. The mean is represented by a star. This demonstrated that the baseline failure rate did not influence the outcome of the experiment.

FIG. 17A. The amplitudes from all synaptic events were plotted on a frequency histogram at baseline in α3 KO mice (from FIG. 16A). The most commonly occurring events are under −100 pA with the entire function fit by the sum of three Gaussian functions.

FIG. 17B. The amplitudes from all synaptic events were plotted on a frequency histogram at baseline in α3 KO mice (from FIG. 16A). The full range of events up to −1000 pA are shown.

FIG. 17C. The amplitudes from all synaptic events plotted on a frequency histogram post-HSF from α3 KO mice (from FIG. 16B). The most commonly occurring events were under −100 pA with the entire function fit by the sum of three Gaussian functions.

FIG. 17D. The amplitudes from all synaptic events were plotted on a frequency histogram post-HSF from α3 KO mice (from FIG. 16B). The full range of events up to −1000 pA are shown.

FIG. 17E. Scatter plot illustrating the decay kinetics for all synaptic events plotted against their amplitude at baseline from α3 KO mice (from FIG. 16A). The graph shows the most commonly occurring events have decay kinetics less than 20 ms.

FIG. 17F. Scatter plot illustrating the decay kinetics for all synaptic events plotted against their amplitude at baseline from α3 KO mice (from FIG. 16A). The graph shows the full range of amplitudes and decays measured.

FIG. 17G. Scatter plot illustrating the decay kinetics for all synaptic events plotted against their amplitude post-HSF from α3 KO mice (from FIG. 16B). The graph shows the most common events have decay kinetics less than 20 ms.

FIG. 17H. Scatter plot illustrating the decay kinetics for all synaptic events plotted against their amplitude post-HSF from α3 KO mice (from FIG. 16B). The graph shows the full range of amplitudes and decays measured.

FIG. 18. Schematic showing how α3 KO mice are characterized by a complete loss of iLTP which reveals more clearly the pronounced iLTD.

FIG. 19A. Graph showing a representative raw trace of voltage clamped inhibitory events from a Fmr1 KO stellate cell (cell no. 20210210p1) at baseline.

FIG. 19B. Graph showing a representative raw trace from a Fmr1 KO STELLATE cell (cell no. 20210210p1) post-HFS.

FIG. 19C. Scatter plot illustrating the failure rate for the cells of FIG. 19A at baseline.

FIG. 19D. Scatter plot illustrating the failure rate for the cells of FIG. 19B post-HFS.

FIG. 19E. Summary graph of the raw failure rate percentages for all cells at baseline compared to post-HFS. The mean is represented by a star. The graph shows that all cells increased their failure rate post-HFS.

FIG. 19F. Graph showing the initial failure rate at baseline for all cells compared to how much that cell changed post-HFS. The mean is represented by a star. This revealed that the baseline failure rate did not influence the degree to which cells changed post-HFS.

FIG. 20A. The amplitudes from all synaptic events from all cells (from FIG. 19A) were plotted on a frequency histogram during the baseline. The graph illustrates the most commonly occurring events are under −100 pA with the entire function fit by the sum of three Gaussian functions.

FIG. 20B. The amplitudes from all synaptic events from all Fmr1 KO cells (from FIG. 19A) were plotted on a frequency histogram during the baseline. The graph shows the full range of amplitudes across cells (up to −3500 pA).

FIG. 20C. The amplitudes from all synaptic events from all Fmr1 KO cells (from FIG. 19B) were plotted on a frequency histogram post-HSF. The graph illustrates the most events under −200 pA with the entire function fit by the sum of three Gaussian functions.

FIG. 20D. The amplitudes from all synaptic events from all cells (from FIG. 19B) were plotted on a frequency histogram post-HSF. The graph shows the full range of amplitudes across cells.

FIG. 20E. Scatter plot of the decay kinetics of all synaptic events from Fmr1 KO cells plotted against their amplitude at baseline. The graph illustrates the most commonly occurring events have decay kinetics of less than 20 ms.

FIG. 20F. Scatter plot of the decay kinetics of all synaptic events plotted against their amplitude at baseline. The plot illustrates the full range of amplitudes and decay kinetics observed.

FIG. 20G. Scatter plot of the decay kinetics of all synaptic events plotted against their amplitude post-HSF. The highlights the most commonly occurring events under −500 pA.

FIG. 20H. Scatter plot of the decay kinetics of all synaptic events plotted against their amplitude post-HSF. The plot illustrates the full range of amplitudes and decay kinetics observed.

FIG. 21A. Graph showing the time latency for all synaptic events measured and plotted at baseline in Fmr1 KO cells.

FIG. 21B. Graph showing the time latency for all synaptic events measured and plotted post-HSF in Fmr1 KO cells.

FIG. 21C. Graph showing the time latency of all synaptic events plotted against their amplitude at baseline in Fmr1 KO cells.

FIG. 21D. Graph showing the time latency of all synaptic events plotted against their amplitude post-HSF in Fmr1 KO cells.

FIG. 22. Schematic illustrating the mechanism of how Fmr1 KO mice lack iLTP but possess an enhanced iLTD.

FIG. 23A. Voltage-clamp recordings of raw GABAR synaptic events from a Fmr1 KO stellate cell (cell no. 20210430p1) in the presence of 10 μM external 2-Methyl-6-(phenylethynyl)-pyridine (MPEP) at baseline.

FIG. 23B. Voltage-clamp recordings of raw trace of a Fmr1 KO stellate cell (cell no. 20210430p1) in the presence of 10 μM external MPEP post-HFS.

FIG. 23C. Scatter plot illustrating the cells from FIG. 23A, at baseline, illustrating the failure rate.

FIG. 23D. Scatter plot illustrating the cells from FIG. 23B, post-HSF, illustrating the failure rate.

FIG. 23E. Summary graph of the raw failure rate percentages for all cells at baseline and post-HFS. The mean is represented by a star. On average, all cells displayed the little change in the failure rate before and after induction of HFS.

FIG. 23F. Graph showing the initial failure rate at baseline for each cell compared to how much that cell changed post-HFS. The mean is represented by a star. The graph demonstrates that the baseline failure rate did not influence the outcome of the experiment.

FIG. 24A. Frequency histogram plot of all the synaptic events at baseline (from FIG. 23A). The graph illustrates the most commonly occurring events under −100 pA with the entire function fit by the sum of three Gaussian functions.

FIG. 24B. Frequency histogram plot of all the synaptic events at baseline (from FIG. 23A). The graph shows the full range of amplitudes and decays measured.

FIG. 24C. Frequency histogram plot of all the synaptic events post-HSF (from FIG. 23B). The graph illustrates the most commonly occurring events under −100 pA with the entire function fit by the sum of three Gaussian functions.

FIG. 24D. Frequency histogram plot of all the synaptic events post-HSF (from FIG. 23B). The graph shows the full range of amplitudes and decay kinetics measured.

FIG. 24E. Graph showing the decay kinetics from all synaptic events plotted against their amplitude for the baseline condition. The graph illustrates the most commonly occurring events have decay kinetics less than 20 ms.

FIG. 24F. Graph showing the decay kinetics from all synaptic events plotted against their amplitude for the baseline condition. The graph shows the full range of amplitudes and decays measured.

FIG. 24G. Graph showing the decay kinetics from all synaptic events plotted against their amplitude post-HSF. The graph illustrates the most commonly occurring events have decay kinetics less than 20 ms.

FIG. 24H. Graph showing the decay kinetics from all synaptic events plotted against their amplitude post-HSF. The graph shows the full range of amplitudes and decays measured.

FIG. 25A. Bar graph showing the time latencies for all synaptic events measured and plotted at baseline (from FIG. 23A) in Fmr1 KO stellate cells in the presence of 10 μM external MPEP.

FIG. 25B. Bar graph showing the time latencies for all synaptic events measured and plotted post-HSF (from FIG. 23B) in Fmr1 KO stellate cells in the presence of 10 μM external MPEP.

FIG. 25C. Scatter plot showing the time latencies for all synaptic events measured and plotted at baseline (from FIG. 23A) in Fmr1 KO stellate cells in the presence of 10 μM external MPEP.

FIG. 25D. Scatter plot showing the time latencies for all synaptic events measured and plotted post-HSF (from FIG. 23B) in Fmr1 KO stellate cells in the presence of 10 μM external MPEP.

FIG. 26A. Voltage-clamp recordings of raw traces from a Fmr1 KO stellate cell (cell no. 20210706p3) in the presence of sildenafil prior to HFS.

FIG. 26B. Voltage-clamp recordings of raw traces from a Fmr1 KO stellate cell (cell no. 20210706p3) in the presence of sildenafil following to HFS.

FIG. 26C. Scatter plot of the response amplitudes showing the same cell as FIG. 26A prior to HFS.

FIG. 26D. Scatter plot of the response amplitudes showing for the same cell as FIG. 26B post-HFS.

FIG. 26E. Summary graph of failure rates for all cells at baseline and post-HFS (FIGS. 26A-26B). The mean is represented by a star showing that failure rates decreased in all cells.

FIG. 26F. Graph showing the initial failure rate at baseline in all cells compared to the change observed following HFS (from FIGS. 26A-26B). The mean is represented by a star,

FIG. 27A. Frequency histogram plot of the amplitudes from all synaptic events plotted at baseline (FIG. 26A). The graph illustrates that most events are under −100 pA with the entire function fit by the sum of three Gaussian functions.

FIG. 27B. Frequency histogram plot of the amplitudes from all synaptic events at baseline (FIG. 26A). The plot illustrates the full range of amplitudes observed.

FIG. 27C. Frequency histogram plot of the amplitudes from all synaptic events plotted post-HSF (FIG. 26B). The graph illustrates that most events under −100 pA with the entire function fit by the sum of three Gaussian functions.

FIG. 27D. Frequency histogram plot of amplitudes from all synaptic events plotted post-HSF (FIG. 26B). The graph shows the full range of amplitudes observed.

FIG. 27E. Graph showing the decay kinetics from all synaptic events plotted against their peak amplitude (cells from FIG. 26A) at baseline. The graph illustrates that most events have decay kinetics of less than 20 ms.

FIG. 27F. Graph showing the decay kinetics from all synaptic events plotted against their peak amplitude (cells from FIG. 26A) at baseline. The graph shows the full range of amplitudes and decay kinetics.

FIG. 27G. Graph showing the decay kinetics from all synaptic events plotted against their peak amplitude (cells from FIG. 26B) post-HSF. The graph reveals that some synaptic events have decay kinetics slower than 20 ms.

FIG. 27H. Graph showing the decay kinetics from all synaptic events plotted against their peak amplitude (cells from FIG. 26B) post-HSF. The graph shows the full range of amplitudes and decay kinetics.

FIG. 28A. Voltage-clamp recordings of raw traces from a Fmr1 KO stellate cell (cell no. 20210709p1) in the presence of both external MPEP and sildenafil at baseline.

FIG. 288. Voltage-clamp recordings of raw trace of a Fmr1 KO stellate cell (cell no. 20210709p1) in the presence of both external MPEP and sildenafil, post-HFS.

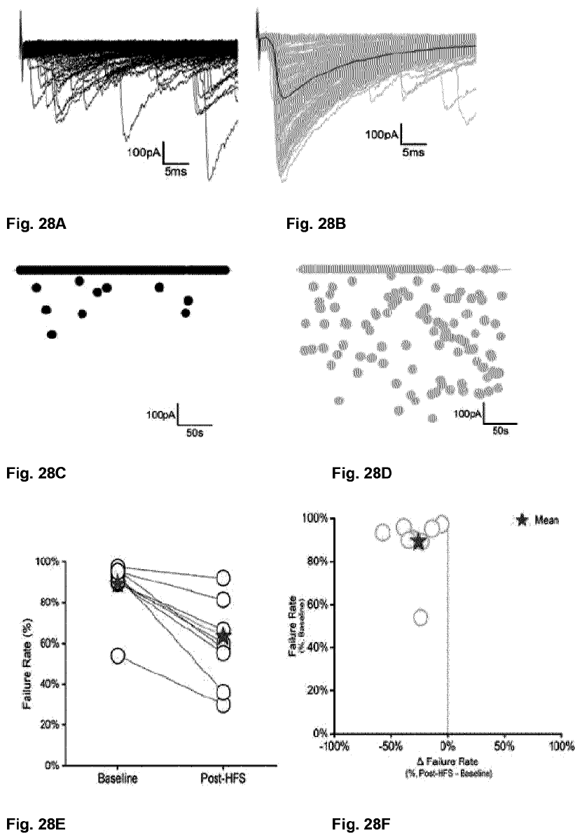

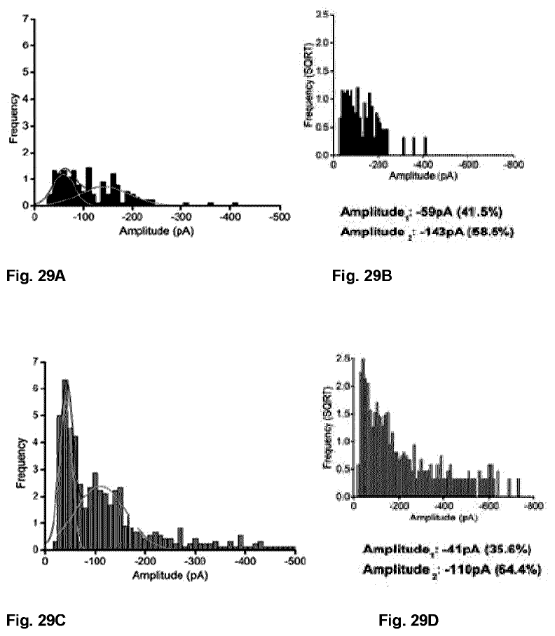

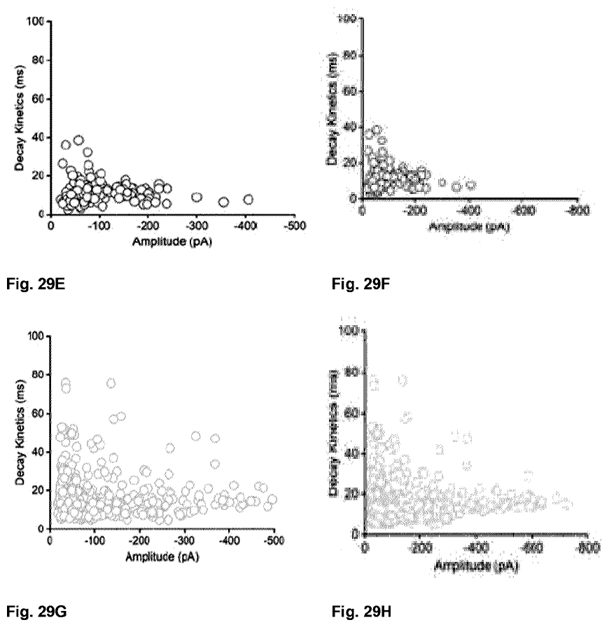

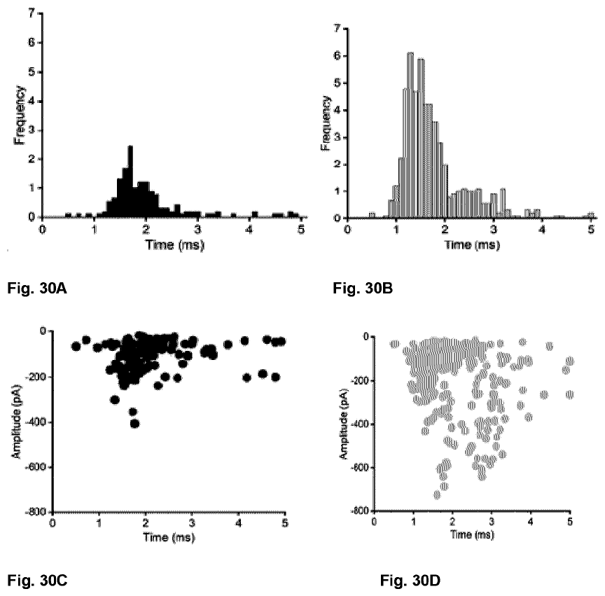

FIG. 28C. Scatter plot of the response amplitudes for the same cells as FIG. 28A at baseline.

FIG. 28D. Scatter plot of the response amplitudes for the same cells as FIG. 28B post-HSF.

FIG. 28E. Summary graph of failure rates for all cells at baseline and post-HFS (FIGS. 28C-28D). The mean is represented by a star showing that failure rates decreased in all cells.

FIG. 28F. Graph showing the initial failure rate at baseline in all cells compared to the change observed following HFS. The mean is represented by a red star. The graph confirms that the initial failure rate did not influence how much that cell would potentiate.

FIG. 29A. Frequency histogram plot of amplitudes of all synaptic events were plotted at baseline (cells from FIG. 28A). The graph shows that most events were under −100 pA in amplitude which was fitted by two Gaussians.

FIG. 29B. Frequency histogram plot of amplitudes of all synaptic events were plotted at baseline (cells from FIG. 28A). The graph shows the full range of amplitudes observed.

FIG. 29C. Frequency histogram plot of amplitudes of all synaptic events plotted post-HSF (cells from FIG. 28B) which was fitted by two Gaussians. The graph illustrates a rescue of the large amplitude synaptic events and promotion of the small amplitude events post-HFS.

FIG. 29D. Frequency histogram plot of amplitudes of all synaptic events plotted post-HSF (cells from FIG. 28B). The graph shows the full range of amplitudes observed.

FIG. 29E. Graph showing the decay kinetics of all synaptic events plotted against their peak amplitude (cells from FIG. 28A) at baseline. The graph illustrates that almost all events are under −500 pA.

FIG. 29F. Graph showing the decay kinetics from all synaptic events plotted against their peak amplitude (cells from FIG. 28A) at baseline. The graph shows the full range of amplitudes and decay kinetics.

FIG. 29G. Graph showing the decay kinetics from all synaptic events plotted against their peak amplitude (cells from FIG. 28B) post-HSF. The graph illustrates the events under −500 pA only.

FIG. 29H. Graph showing the decay kinetics from all synaptic events plotted against their peak amplitude (cells from FIG. 28B) post-HSF. The graph shows the full range of amplitudes and decay kinetics.

FIG. 30A. Graph showing the time latencies from all synaptic events measured and plotted at baseline (cells from FIG. 28A).

FIG. 30B. Graph showing the time latencies from all synaptic events measured and plotted post-HSF (cells from FIG. 28B).

FIG. 30C. Scatter plot of the response amplitudes for the same cell as FIG. 30A at baseline.

FIG. 30D. Scatter plot of the response amplitudes for the same cell as FIG. 30B post-HSF.

### DETAILED DESCRIPTION

The present disclosure is based on the understanding that the modulation of the signaling pathways triggered by NMDARs expressed by stellate cells can be beneficial for the mitigation of symptom(s) associated with FXS. As shown in FIG. 1, the synaptic release of the neurotransmitter, L-glutamate (L-Glu), activates postsynaptic NMDA receptors which transport external Ca²⁺ into the cytosol of WT stellate cells (i.e. neuron). Elevated Ca²⁺ stimulates a bifurcating pathway that activates neuronal nitric oxide synthase (nNOS), which converts arginine into nitric oxide (NO), but also activates CaM kinase II (not shown on FIG. 1). NO acts on guanylate cyclase (GC) to generate cGMP (not shown on FIG. 1) which promotes both the strengthening of GABAA receptor inhibitory synapses (iLTP) via protein kinase C (PKC). The NO generated by nNOS also causes vasodilation of nearby capillaries. Activated CaM kinase II acts on a separate pathway that leads to the modulation of voltage-gated Na⁺ channels to promote an increase in action potential firing in stellate cells.

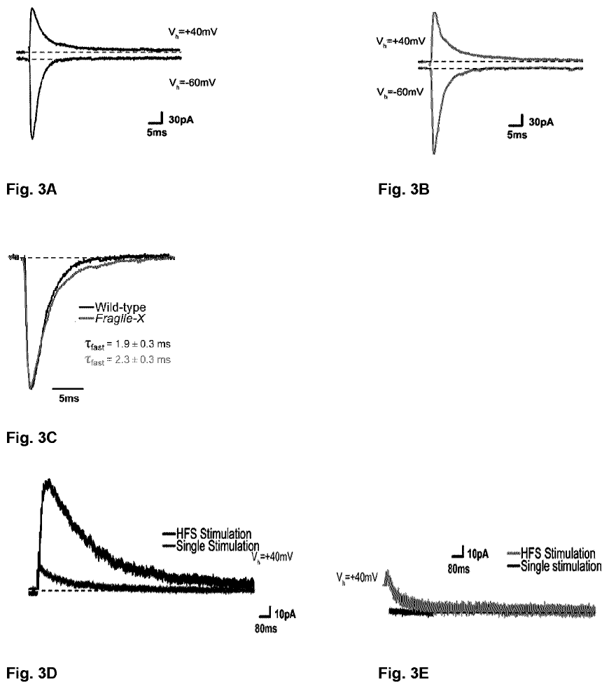

As shown in FIG. 3E, the NMDA receptor response is almost completely absent in stellate cells from Fmr1 KO mice (e.g., a mouse model of FXS). Consequently, there is insufficient activation of guanylate cyclase and CaM kinase II and thus, the strengthening of GABAA receptor plasticity, modulation of voltage-gated Na⁺ channels and the vasodilation of nearby capillaries is lost. As also shown in the Example, by inhibiting PDE5 that breakdowns cGMP, sildenafil restores the strengthening of GABAA receptor inhibitory synapses and triggers intrinsic plasticity in stellate cells of FXS mice.

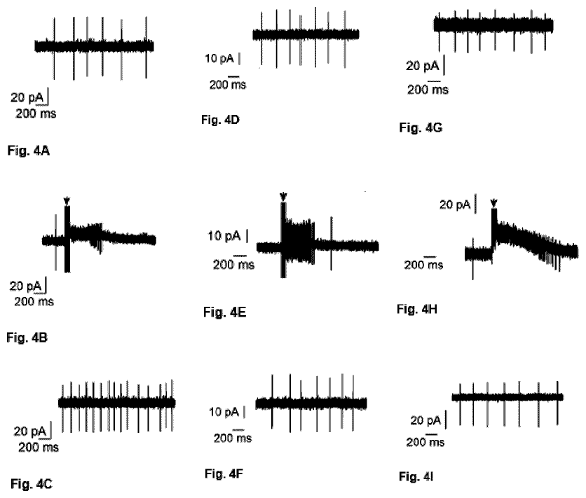

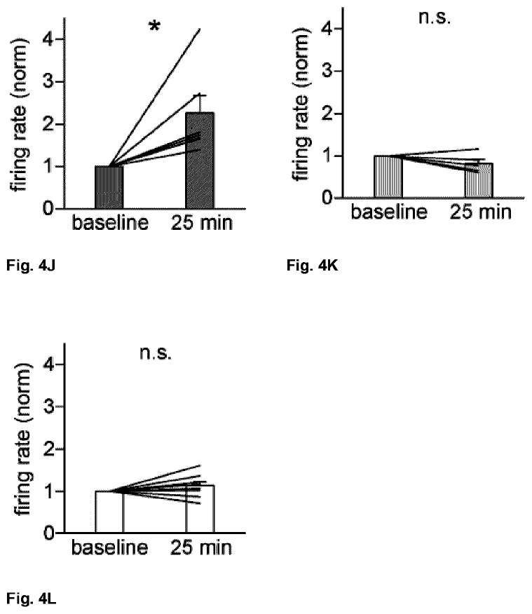

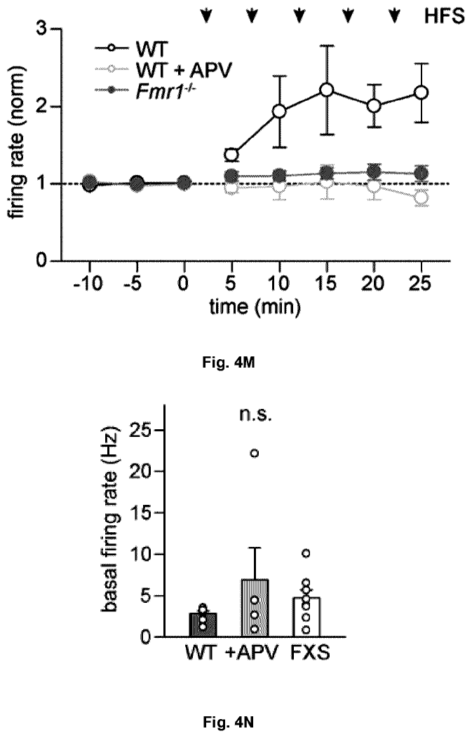

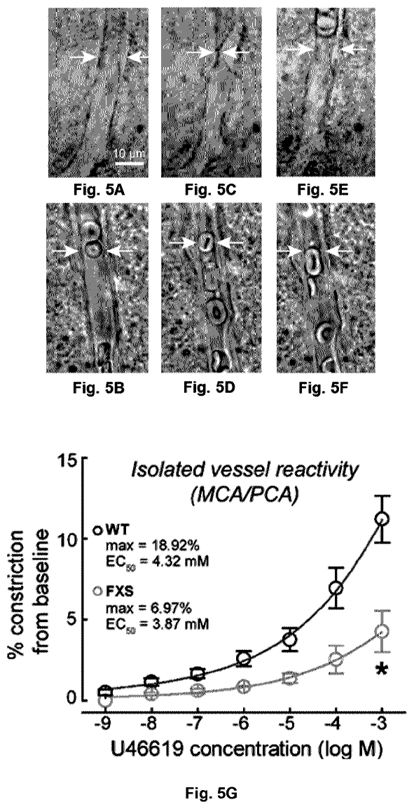

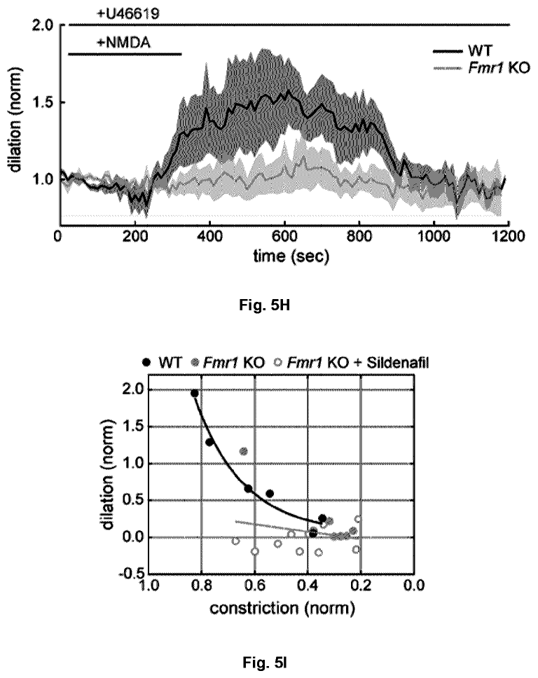

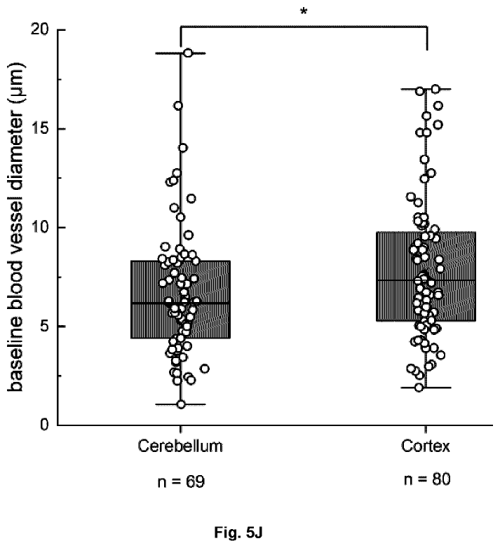

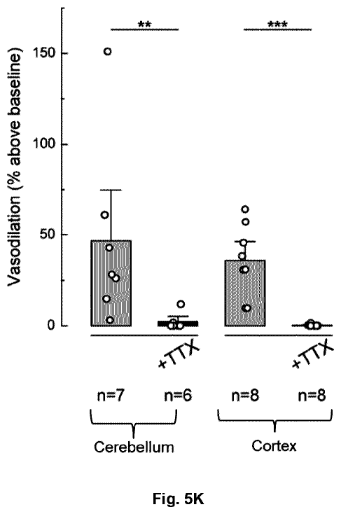

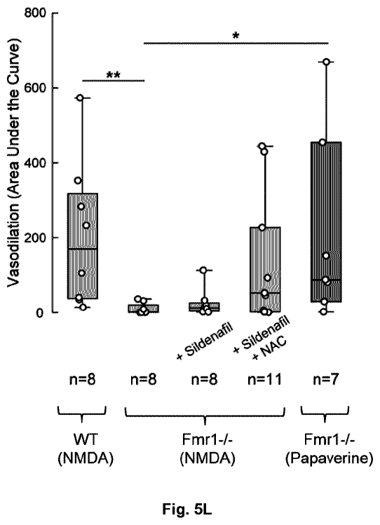

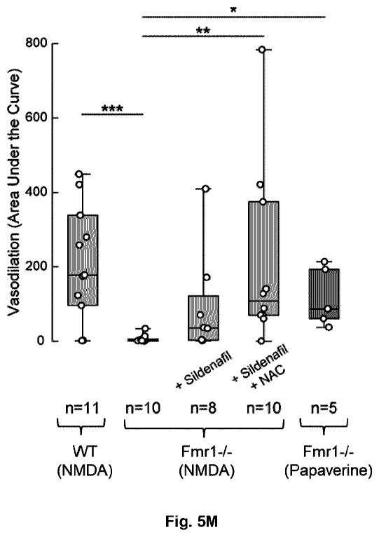

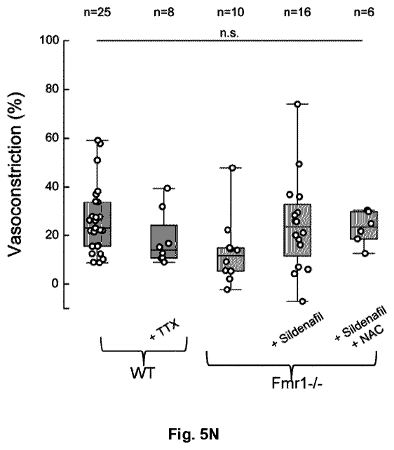

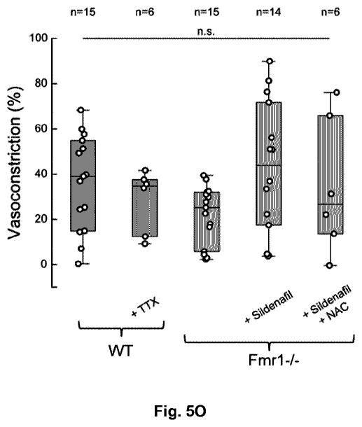

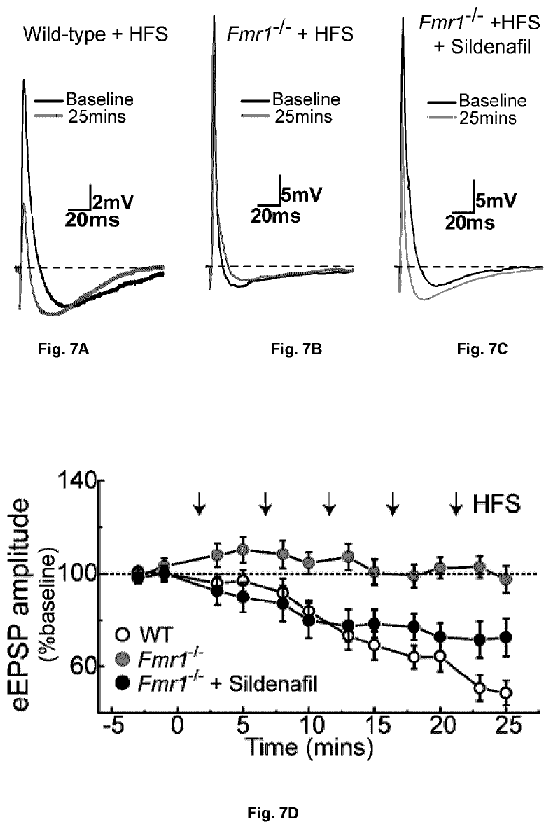

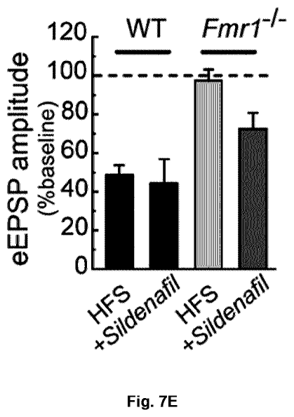

NMDAR-NO signaling is found throughout the developing and adult brain and plays important roles in the formation and development of synaptic organization and synaptic plasticity, strengthening inhibitory GABAergic synapses, and different behavioral traits such as learning and memory. In the Example, it is shown that FXS brain cells also have much diminished signaling by extrasynaptic NMDARs (see FIGS. 3A-3E). The weak NMDAR response in the FXS brain means that learning mechanisms driven by NMDARs in the brain are lost, namely intrinsic plasticity of neuronal firing (see FIGS. 4A-4N and Alexander & Bowie 2021) and long-term potentiation of inhibition (or iLTP) (see FIGS. 7A-7E and Larson et al. 2020). The lack of NMDA response also causes a loss of NO-mediated vasodilation of local capillaries (FIGS. 5A-5I). Given these findings, without wishing to be bound by theory, the prolongation of the half-life of cGMP in neuronal and vascular tissue of the FXS brain cells restores the downstream effects of NMDAR-NO signaling and mitigates symptoms related to NMDAR-NO signaling hypofunction therein.

### Abbreviations

ASR: acoustic startle response
APV: (2R)-amino-5-phosphonovaleric acid
AMPA: α-amino-3-hydroxy-5-methyl-4-isoxazolepropionic acid
AMPAR: AMPA receptor
BAPTA: 1,2-bis(o-aminophenoxy)ethane-N,N,N′,N′-tetraacetic acid)
CAMP: cyclic adenosine monophosphate
cGMP: cyclic guanosine monophosphate
eNOS: endothelium nitric oxide synthase
EPSP: excitatory postsynaptic potentials
FXS: Fragile X Syndrome
GABA: γ-aminobutyric acid
GABAR: γ-aminobutyric acid receptor
GABAA: γ-aminobutyric acid type A
GABAAR: γ-aminobutyric acid type A receptor
GC: granule cells
HFS: High frequency stimulation
iLTP: long-term potentiation of inhibition
I.P.: intraperitoneal
IPSC: inhibitory postsynaptic current
KO: Knock out
STELLATE: molecular layer interneuron
NMDAR: N-methyl-D-aspartate receptor
MPEP: 2-Methyl-6-(phenylethynyl)-pyridine
nNOS: neuronal nitric oxide synthase
PDE: phosphodiesterase
PF: parallel fiber
PPI: Prepulse inhibition
ROS: reactive oxygen species
WT: wild-type
   Phosphodiesterases that Degrade cGMP and their Inhibitors

The present disclosure thus provides one or more inhibitor of one or more phosphodiesterase (PDE) to mitigate one or more symptoms of FXS. The PDE that is being inhibited is capable of cGMP degradation and optionally of cAMP degradation. cGMP has been previously shown to play an important role in calcium homeostasis, signal transduction (e.g., glutaminergic, cholinergic and GABAergic) and other physiological responses in the brain (e.g., blood vessel dilation) (Domek-Łopacińska et al., 2005). Here, the present disclosure demonstrates that, surprisingly, many of these same processes and pathways are defective in FXS, including glutamatergic and GABAergic signaling and cerebral blood vessel dilation (leading to abnormal cerebral blood flow). The present disclosure provides, for the first time, a link between a hypofunction in cGMP signaling and FXS.

Phosphodiesterases are enzymes that are capable of hydrolyzing phosphodiester bonds. While there are several categories of phosphodiesterases, which can be differentiated based on the nature of the substrates that they target, those that degrade cyclic nucleotides, like cGMP and cAMP, are particularly important from a clinical standpoint, as they are often targets for pharmacological inhibition due to their unique tissue distribution, structural properties, and functional properties. In the context of the present disclosure, the phosphodiesterase that is being inhibited comprises a cyclic nucleotide phosphodiesterase and, specifically, those that hydrolyze cGMP.

At least eleven different gene families of PDEs have been identified and characterized in mammals (PDE1 to PDE11) based on their molecular sequence, kinetics, regulation and pharmacological characteristics. Some of these families have more than one member (i.e. isoform) each of which is encoded by different genes (e.g., PDE4A, PDE4B, PDE4C and PDE4D). The families themselves and the isoforms within the respective family have varying substrate preferences for cAMP and cGMP. PDE families 1, 2, 3 and 10 hydrolyze both cGMP and cAMP; PDE families 4, 7 and 8 preferentially cleave cAMP and PDE families 5, 6 and 9 specifically hydrolyze cGMP.

As used herein, the term “inhibitor of one or more phosphodiesterase” refers to small molecule compounds or biologics that reduce or prevent the breakdown of cGMP by PDEs in neurons, especially stellate cells, thereby inducing cGMP-dependent signaling pathways and physiological processes. In some embodiments, the inhibitor is able to mediate its therapeutic action in a neuron capable of expressing neuronal nitric oxide synthase. In some embodiments, the one or more inhibitor (after having been administered to the individual) is capable of facilitating in at least one brain region (the cerebellum for example): (a) intrinsic plasticity via a sodium channel; (b) vasodilation; and/or (c) GABAergic inhibitory synaptic plasticity. This facilitation can be observed when comparing the same brain region in a control individual. This control individual may be the individual prior to treatment. The control individual may also be a distinct individual (or a population of distinct individuals) having been diagnosed with FXS but not having been administered with the one or more PDE inhibitor. In some embodiments, the inhibitor or the combination of inhibitors is or comprises a non-selective phosphodiesterase inhibitor. In yet another embodiment, the inhibitor or the combination of inhibitors is or comprises a selective phosphodiesterase inhibitor.

Defective signaling pathways in FXS have been presently identified in the cerebellum. Although traditionally associated with motor function, the cerebellum has been found to be an important brain region in FXS since it has been strongly linked to many aspects of FXS and autistic disorders including eye-blink conditioning, disrupted dendritic spines and exaggerated synaptic plasticity, such as LTD. In addition, the cerebellum has an unappreciated role in guiding non-motor circuitry that influences cognitive development especially those concerned with cognition and affect. A novel plasticity mechanism was identified in the cerebellum by which reactive oxygen species (ROS) strengthen inhibitory GABAergic synapses of molecular layer interneurons (stellate cells) and granule cells (GCs). It was found that ROS-mediated synaptic plasticity is disrupted in these cell-types since disrupted levels of ROS are found in Fmr1 KO mice and FXS patients. The term “molecular layer interneuron” (MLI) refers to stellate cells. In the present disclosure all experiments were conducted with stellate cells. The broader term MLI, which also includes basket cells, in the present disclosure only refers to stellate cells.

The present disclosure investigated the stellate cells of the cerebellum. NMDARs act as a master switch to trigger a long-term increase in neuronal firing, by modifying voltage-gated Na⁺ channels, whilst strengthening inhibitory GABAergic synapses through the activity of neuronal nitric oxide synthase (nNOS) and cytosolic ROS. Since GCs are the only other nNOS positive (nNOS*) neurons found in the cerebellum, NMDARs of these cell types are expected to similarly promote intrinsic plasticity and strengthen GABAR synapses. Two important observations were made from the present experimental results. First, it was shown that in a preclinical model of Fragile X syndrome, the Fmr1 KO mouse, stellate cells exhibit marked deficits in both the long-term potentiation (iLTP) and depression (iLTD) of inhibitory GABAergic synapses. Second, it was shown that inhibition of phosphodiesterase 5 with sildenafil corrects deficits in iLTP and that inhibition of mGluR5 receptor signaling with MPEP corrects deficits in iLTD. The present disclosure therefore establishes a rationale for treating patients of FXS with a combination therapy of PDE inhibitors and a mGluR5 blocking agent.

In some embodiments, the one or more phosphodiesterase comprises at least one phosphodiesterase that is selective for cGMP (e.g. PDE 5, PDE6, and PDE 9). In embodiments in which the inhibitor or combination of inhibitors is intended to mediate their therapeutic actions on neurons that are capable of expressing/are expressing nNOS, the phosphodiesterase comprises PDE5. In some embodiments, the inhibitor comprises a PDE5 inhibitor alone or in combination with at least one of a PDE1, PDE2 or PDE10 inhibitor. Known non-selective PDE5 inhibitors include, without limitation, pentoxifylline (Trental®, Pentoxil) as well as its pharmaceutically acceptable salts. Known selective PDE5 inhibitors include, without limitation sildenafil (Viagara®), avanafil (Stendra®), tadalafil (Cialis®), vardenafil (Staxyn®, Levitra®), udenafil (Zydena®), mirodenafil (Mvix®), iodenafil, zaprinast, icariin as well as their pharmaceutically acceptable salts.

In some embodiments, the one or more phosphodiesterase comprises a phosphodiesterase capable of hydrolyzing both cGMP and cAMP (e.g. PDE 1, PDE 2, PDE 3 and PDE 10). In embodiments in which the inhibitor or combination of inhibitors is intended to mediate their therapeutic actions on neurons that are capable of expressing/are expressing nNOS, the phosphodiesterase comprises PDE1, PDE2 and PDE10. In some embodiments, the inhibitor comprises a PDE1 inhibitor alone or in combination with at least one of a PDE2, PDE5 or PDE10 inhibitor. Known non-selective inhibitors of PDE1 include, but are not limited to, dipyridamole (Persantine®). Known selective inhibitors of PDE1 include, but are not limited to, vinpocetine (Cavinton®) or its pharmaceutically acceptable salt. In some embodiments, the inhibitor comprises a PDE2 inhibitor alone or in combination with at least one of a PDE1, PDE5 or PDE10 inhibitor. Known non-selective inhibitors of PDE2 include, but are not limited to, tofisopam (Emandaxin®, Grandaxin®) or its pharmaceutically acceptable salt. In some embodiments, the inhibitor comprises a PDE10 inhibitor alone or in combination with at least one of a PDE1, PDE2 or PDE5 inhibitor. Known non-selective inhibitors of PDE10 include, but are not limited to, ibudilast (Ketas®, Pinatos®), Eyevinal®) and tofisopam (Emandaxin®, Grandaxin®) as well as their pharmaceutically acceptable salts. Known selective inhibitors of PDE10 include, but are not limited to, papaverine (Pavabid®, Pavagen®) as well as its pharmaceutically acceptable salt.

As indicated herein, the inhibitor or the combination of inhibitors can be provided as a pharmaceutically acceptable salt. This expression refers to conventional acid-addition salts or base-addition salts that retain the biological effectiveness and properties of the therapeutic agent described herein. They are formed from suitable non-toxic organic or inorganic acids or organic or inorganic bases. Sample acid-addition salts include those derived from inorganic acids such as hydrochloric acid, hydrobromic acid, hydroiodic acid, citric acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium and, quaternary ammonium hydroxides, such as e.g., tetramethylammonium hydroxide. The chemical modification of an agent into a salt is a well-known technique which is used in attempting to improve properties involving physical or chemical stability, e.g., hygroscopicity, flowability or solubility of the inhibitor(s).

The inhibitor or combination of inhibitors is intended to be provided to the individual in a therapeutically effective amount. As used in the context of the present disclosure, the term “therapeutically effective amount” refers to a quantity of the one or more PDE inhibitor (i.e. a dose) that is effective in mitigating one or more symptom of FXS when administered to an individual in need thereof. It is also understood herein that a therapeutically effective amount of the one or more inhibitor may be administered in different dosage forms and by different routes, both alone or in combination with other therapeutic agents used to treat FXS symptoms (e.g. anti-anxiety medication, antiepileptic drugs, etc.).

The inhibitor or the combination of inhibitors can be provided as a pharmaceutical composition. When more than one inhibitor is used, the pharmaceutical composition can provide each individual inhibitor in a distinct dosage form or all inhibitors in a single dosage form. The expression “pharmaceutical composition” refers to therapeutically effective amounts (dose) of the inhibitor/combination of inhibitors together with pharmaceutically acceptable diluents, preservatives, solubilizers, emulsifiers, adjuvants and/or carriers.

The pharmaceutical composition can include one or more pharmaceutically acceptable carrier. This term refers to an acceptable carrier or adjuvant that may be administered to a patient, together with a compound of this disclosure, and which does not destroy the pharmacological activity thereof. Further, as used herein “pharmaceutically acceptable carrier” or “pharmaceutical carrier” are known in the art and include, but are not limited to, 0.01-0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

The dosage form of the PDE inhibitor or the combination of PDE inhibitors may be a tablet, a pill, a capsule, a syrup, a film, a liquid solution, a liquid suspension, a powder, a paste or an aerosol. The route of administration of the PDE, which will depend to a large extent on the dosage form, may be oral, sublingual, buccal, parenteral, topical, intranasal or ophthalmic.

In some embodiments, a therapeutically effective amount of at least two distinct phosphodiesterase inhibitors is administered in order to mitigate the one or more symptoms of FXS in the individual. It should be understood that the at least two or more phosphodiesterase inhibitors may be administered separately or in combination. Further, the at least two phosphodiesterase inhibitors may target the same PDE family or may target, whether selectively or non-selectively, different PDE families.

The PDE inhibitor or the combination of PDE inhibitors can be used to mitigate one or more FXS symptom in an “individual in need thereof”. The expression refers to an individual displaying one or more symptom associated with FXS. In some embodiments, the individual has been previously diagnosed with FXS before being administered with the PDE inhibitor or the combination of PDE inhibitors. Alternatively or in combination, the FXS symptoms of the individual in need thereof are measured before and/or after having been administered one or more dose of the PDE inhibitor or the combination of PDE inhibitors. In some embodiments, the individual is a human. In some embodiments, the individual is a child. In some embodiments, the individual is a baby. In some embodiments, the individual is a newborn.

The PDE inhibitor or the combination of PDE inhibitors are to mitigate at least one symptom of FXS. The expression “mitigation of at least one FXS symptom” refers to the ability of the method and/or the PDE inhibitors described herein to limit the development, progression and/or symptomology of FXS. The symptoms comprise any clinical symptoms, whether severe or mild, found in individuals with FXS. The symptoms associated with FXS include, but are not limited to: hyperactivity, male aggression, anxiety, a learning deficit (such as, for example, a reversal learning deficit and/or a cued & contextual fear conditioning), a memory deficit (such as, for example, a spatial memory deficit and/or a cued & contextual fear conditioning), a sensory deficit (such as, for example a sensorimotor skill deficit, a sensory sensitivity deficit and/or a startle response), sleep abnormalities and/or repetitive behavior. Individuals with FXS also display physical traits such as an elongated face, protruding ears and macroorchidism (enlarged testes) and exhibit stereotypic behavior, such as hand-flapping, and social anxiety. Moreover, almost half of all individuals with FXS have features associated with autism.

mGluR5 Receptor Blocking to Prevent or Treat FXS

The present disclosure provides an unprecedented understanding of how plasticity of GABAR synapses is disrupted in FXS. Experiments on WT and α3 KO mice revealed that small amplitude and slow decaying α3-containing GABARs are essential for promoting the synaptic connectivity of neurons, necessary for iLTP. Unexpectedly, the observations made herein included finding a modest but appreciable iLTD of the large amplitude and fast decaying α1-containing GABARs. α3-mediated GABAR synaptic strengthening is completely lost in Fmr1 KO mice whereas α1 GABAR-mediated iLTD is significantly enhanced. Importantly, inhibition of PDE5 to prolong the half-life of cGMP together with inhibition of mGluR5 receptor signaling completely prevent the exaggerated synaptic depression and restore normal iLTP synaptic strengthening. Taken together, the present findings provide insight into the specific profile that different subtypes of GABARs fulfill in the WT mouse and their dysfunction in central nervous system (CNS) disease.

### α3-Containing GABARs Promote Synaptic Strengthening by Occupying Silent Synapses

The present disclosure establishes that α3-containing GABARs are integrally involved in iLTP. The data presented herein shows that extrasynaptic NMDAR stimulation triggers the NO/cGMP signalling pathway to promote the selective insertion of small amplitude and slow decaying α3-containing GABAR mIPSCs. Minimal stimulation experiments described herein establish the proposal that α3-containing GABARs occupy silent inhibitory synaptic sites. The pathway activated by NMDARs is mediated by NO/cGMP signaling given the effectiveness of the pharmacological inhibition of PDE5 by sildenafil.

α1-Containing GABARs Undergo Synaptic Depression During iLTP

α1 GABAR synapses undergo synaptic depression or iLTD following induction after HFS. It was previously thought that glutamatergic transmission can promote iLTP via the insertion of α3-containing GABARs and it was assumed that α1 synapses remain unaffected. Surprisingly, the present disclosure demonstrated that 1 synapses are also dynamically regulated and can undergo activity-dependent iLTD. Accordingly, one of the observations of the results presented herein is that the loss of large amplitude and fast decaying events post-HFS is due to α1-containing GABARs.

In the molecular layer of the cerebellum, the majority of cell types express mostly α1, some α3, but α2 GABARs have also been reported. The role that α2-containing GABARs may be playing in synaptic strengthening or depression remains to be understood. Typically, α2-containing GABARs are largely enriched at the axonal initial segment (AIS) and act to control the excitability of the cell by regulating the generation of action potentials. In electrophysiology experiments, α2-containing GABARs exhibit quite similar characteristics to α1-containing GABARs, therefore it is possible that the iLTD observed in the present disclosure could involve α2 GABARs. Nonetheless, α1-containing GABARs are most commonly expressed in the cerebellum and it can be concluded that they are largely responsible for iLTD.

Fmr1KO Mice Experience mGluR Mediated Enhanced ILTD Post-HFS

Uncovering that Fmr1 KO mice are subject to enhanced iLTD is an unexpected finding. While LTP mechanisms for glutamatergic synapses in Fmr1 KO mice have been well documented, inhibitory LTP is much less discussed and researched. LTD is exaggerated in Fmr1 KO mice due to the overactivity of Group 1 (Gp1) mGluRs. During the inhibitory synaptic strengthening, Fmr1 KO mice experience enhanced synaptic depression mediated by mGluRs. First, there is a lack of iLTP in these mice, since a reduction in the failure rate was not observed, but rather a reduction in global synaptic activity was observed. This enhanced iLTD phenotype was able to be rescued with MPEP, confirming that Gp1 mGluRs are responsible for the depression of large amplitude synaptic events (see Example 2).

### Treatments for FXS Utilizing a Combinational Therapeutic Approach

Pharmacological block of mGluR5 activity in Fmr1 KO mice and its prevention of iLTD is a novel finding since mGluR-LTD has only been shown for excitatory glutamatergic systems. The present results provide a first demonstration of how Gp1 mGluR activity can affect GABAergic transmission and plasticity. Although MPEP was sufficient in blocking the enhanced iLTD in Fmr1 KO mice there are a few limitations when assessing the translational value of this therapeutic for humans. The mGluR theory of FXS has been well documented and described, and clinical trials were even tested in human patients. In recent years many mGluR5 antagonists have been studied in clinical trials on individuals with FXS, for example mavoglurant. Unfortunately, this trial ended due to negative results and limitations surrounding the drug's efficacy in FXS patients. There are certainly challenges when it comes to translating positive findings from a preclinical rodent model to the disease in humans. There are uncertainties about age of treatment onset, dosage and durations of treatment, differences in pharmacokinetics and pharmacodynamics, side effects, and biomarkers of CNS improvement. Therefore, no treatment has yet to be approved for FXS, because all approaches have been deemed ineffective in treating all the synaptic and behavioural deficits noticed in these individuals. While these failed clinical trials do not invalidate the mGluR theory, they argue that there is much more to learn about the pathology of FXS, in order to develop treatments that will actually be beneficial for patients.

Considering the extensive targets of FMRP, an important aspect has often been neglected when looking for a useful drug treatment. Current research has focused consistently on targeting only one pathway or receptor at a time. Although, targeting multiple pathways simultaneously, or a combination therapy, may be needed to adequately ameliorate FXS. For example, a combination therapy of two or more drugs may be a good way to combat shortcomings from individual drugs on their own.

The present disclosure provides, in some embodiments, treatment utilizing a blocking agent of the mGluR receptor such as MPEP and an inhibitor of phosphodiesterase such as sildenafil for the treatment or prevention of enhanced iLTD and/or for restoring iLTP. In this embodiment, the treatment or prevention method directly targets two defective bifurcating pathways in FXS. On its own, preventing excessive Gp1 mGluR activity, or using sildenafil can only rescue one aspect of the Fmr1 KO phenotype. Improved efficacy is achieved when combining both drugs (mGluR blocking agent and phosphodiesterase inhibitor) to augment synaptic strengthening in a subject in need thereof as demonstrated in Example 2 in the animal model.

As used herein, the term “blocking agent of mGluR” or “blocking agent” for short, refers to a small molecule or a biologic (e.g. antibody and its derivatives) that can inhibit the binding to mGluR such as mGluR5. In preferred embodiments, mGluR is mGluR5. The blocking agent can be an antagonist or a negative allosteric inhibitor. The blocking agent is preferably a negative allosteric inhibitor such as MPEP. In some embodiments, the blocking agent is able to mediate its therapeutic action in a neuron capable of expressing neuronal nitric oxide synthase. In some embodiments, the blocking agent (after having been administered to the individual) is capable of facilitating in at least one brain region (the cerebellum for example) the blocking of ILTD. This facilitation can be observed when comparing the same brain region in a control individual. This control individual may be the individual prior to treatment. The control individual may also be a distinct individual (or a population of distinct individuals) having been diagnosed with FXS but not having been administered with the blocking agent.

In some embodiments, the mGluR blocking agent is a mGluR5 blocking agent and is selected from the non-limitative example list of: 2-Methyl-6-(phenylethynyl)-pyridine (MPEP), methyl (3aR,4S,7aR)-4-hydroxy-4-[2-(3-methylphenyl)ethynyl]octahydro-1H-indole-1-carboxylate (mavoglurant). N-(3-Chlorophenyl)-N′-(1-methyl-4-oxo-4,5-dihydro-1H-imidazol-2-yl)urea (fenobam), 3-((2-Methyl-1,3-thiazol-4-yl)ethynyl)pyridine (MTEP), 6-methyl-2-(phenylazo)-3-pyridinol (SIB-1757), (E)-2-methyl-6-(2-phenylethenyl)pyridine (SIB-1893), basimglurant (2-chloro-4-{2-[1-(4-fluorophenyl)-2,5-dimethyl-1H-imidazol-4-6-Fluoro-2-(4-(pyridin-2-yl)but-3-yn-1-yl)imidazo(1,2-a)pyridine yl]ethynyl}pyridine), (dipraglurant), 3-fluoro-5-[3-(5-fluoropyridin-2-yl)-1,2,4-oxadiazol-5-yl]benzonitrile (AZD 9272), 2-[(3-Fluorophenyl)ethynyl]-4,6-dimethyl-3-pyridinamine (raseglurant), N-(5-Fluoropyridin-2-yl)-6-methyl-4-(pyrimidin-5-yloxy)picolinamide (VU0424238), GRN-529 ([4-(Difluoromethoxy)-3-[2-(2-pyridinyl)ethynyl]phenyl](5,7-dihydro-6H-pyrrolo[3,4-b]pyridin-6-yl)-(6-Bromopyrazolo[1,5-a]pyrimidin-2-yl)[(1R)-1-methyl-3,4-dihydro-2(1H)-methanone), isoquinolinyl]methanone (remeglurant), (2S)-2-Amino-2-[(1S,2S)-2-carboxycycloprop-1-yl]-3-(xanth-9-yl)propanoic acid (LY-341495). GET73 (4-methoxy-N-[4-(trifluoromethyl)phenyl]methyl]butanamide), arbaclofen ((3R)-4-amino-3-(4-chlorophenyl)butanoic acid), HTL-0014242 ((3-Chloro-5-[6-(5-fluoropyridin-2-yl)pyrimidin-4-yl]benzonitrile)), 2-chloro-N-[2-methoxy-4-(pyridin-2-yldiazenyl)phenyl]benzamide (Alloswitch1), PAM12,4-chloro-N-(6-(pyrimidin-5-yloxy)pyrazin-2-yl)picolinamide (VU-0431316), N-(4,4-dimethylcyclohexyl)pyrido[1′,2′:1,5]pyrazolo[4,3-d]pyrimidin-4-amine [VU-0467558), VU-0463841 (1-(5-chloropyridin-2-yl)-3-(3-cyano-5-fluorophenyl)urea), AP-612, LCGM-10, (3-fluorophenyl)[2-(5-fluoropyridin-2-yl)]-6,7-dihydoro[1,3]oxazolo[4,5-c]pyridin-5(4H)-yl]methanone (DSR-98776), EPX-105287, (as)-α-Amino-α-[(1R,2R)-2-carboxycyclopropyl]-9H-xanthene-9-propanoic acid (LY-344545), MRZ-8676 (6,6-dimethyl-2-(2-phenylethynyl)-7,8-dihydroquinolin-5-one), 3-((4-(4-chlorophenyl)-7-fluoroquinolin-3-yl)sulfonyl)benzonitrile (RGH-618), 5-(3-chlorophenyl)-3-[(1R)-1-[(4-methyl-5-pyridin-4-yl-1,2,4-triazol-3-yl)oxy]ethyl]-1,2-oxazole (AZD-2066), AZD-2516, AZD-6538 (6-[5-(3-cyano-5-fluorophenyl)-1,2,4-oxadiazol-3-yl]pyridine-3-carbonitrile), and (RS)-α-methyl-4-carboxyphenylglycine ((RS)-MCPG). Other mGluR5 can be used for example those described in US2010273772.

The blocking agent, can be formulated in combination with the phosphodiesterase inhibitor as a pharmaceutically acceptable salt. The blocking agent and the phosphodiesterase inhibitor can also be formulated in different pharmaceutical salts/compositions and administered separately to the subject in need thereof. The combination of mGluR blocking agent and phosphodiesterase inhibitor is provided to an individual in a therapeutically effective amount. The therapeutically effective amount of phosphodiesterase inhibitor can advantageously be lower in the combinatorial therapy with the blocking agent. Indeed, one of the advantage of the combination therapy is that each drug in the combination can be administered at a lower dose compared to the dosage for the drug alone while still obtaining an improved efficacy. The lower dose is advantageous because it reduces the risk of side effects for example.

The dosage form of the combination therapy may be one or more tablets, one or more pills, one or more capsules, one or more syrup, one or more films, one or more liquid solutions or suspensions, one or more powder, one or more pastes, one or more aerosols, or combinations thereof. The route of administration of the PDE and blocking agent will depend to a large extent on the dosage form, which may be oral, sublingual, buccal, parenteral, topical, intranasal or ophthalmic.

### Treatment of Other Conditions Similar to FXS

Fragile X syndrome (FXS), GRIN disorder, SynGAP1 intellectual disability and Phelan-McDermid syndrome are all neurodevelopmental disorders that share a number of clinical features, most notably deficits in an individual's intellectual ability. Despite this clinical overlap, all four disorders are due to different molecular deficits but nevertheless may be treated by the same combination of drugs.

FXS results from the silencing of the Fmr1 gene which encodes the RNA binding protein, Fragile X Messenger Ribonucleoprotein (FMRP). SynGAP1 disorder is caused by mutations in the gene SYNGAP1 which encodes the synaptic scaffolding protein, SynGAP1 (or Synaptic Ras GTPase-activating protein 1). GRIN disorder is caused by mutations in the genes that encode individual subunits of the neurotransmitter receptor protein, N-methyl-D-aspartate receptors (NMDARs). Some cases of Phelan-McDermid syndrome are due to pathogenic variants in the gene that encodes another synaptic protein: Shank3.

Despite the different molecular origins of each disorder, they all share the fact that the proteins involved (i.e. FMRP, SynGAP1, NMDAR and Shank3) are all abundantly found at glutamatergic synapses. Since the present disclosure has demonstrated the treatment of a mouse model of FXS having a profound deficit in signaling by NMDARs at glutamatergic synapses, then that treatment can be extended to treat the loss of SynGAP1, NMDAR subunits and Shank3 since they exhibit similar deficits as Fmrp1 and can be similarly rescued by inhibition of PDE5 with or without a mGluR (e.g. mGluR5) negative allosteric modulator.

SynGAP1 is a key protein that regulates the strengthening of glutamatergic synapses and therefore is likely to impact the NMDAR strengthening in GABAergic synapses, intrinsic excitability as well as regulating the vasodilatory ability of local blood vessels. Likewise, GRIN mutations that cause a reduced global expression of synaptic NMDARs will be expected to cause a complete loss or attenuation of GABAergic and intrinsic plasticity as well as appreciable deficits in neurovascular coupling. Finally, the loss of Shank3 also causes deficits in the morphology and the strength of signaling at glutamatergic synapses which would elicit similar deficits as those identified in the FXS mice.

Taken together, the lack of expression of FMRP, SynGAP1, NMDAR subunits and Shank3 would all be expected to give rise to similar phenotypes at the level of the glutamatergic synapse and, by extension, to an individual's learning ability and/or behavior. The present disclosure therefore provides an explanation for the apparent conundrum whereby all 4 neurodevelopmental disorders mentioned herein have similar clinical features but yet are due to different molecular defects. Therefore, all embodiments relating to Fmrp1 also apply to SynGAP1 ID, GRIN disorders and Phelan-McDermid syndrome and the same therapeutic treatment can be used for all of these disorders.

### Methods for Determining the Usefulness of a Test Agent for the Mitigation of FXS Symptoms

Another aspect of the present disclosure concerns a screening method for determining whether a test agent or a combination of test agents may be capable of mitigating one or more symptoms of FXS. The screening method comprises contacting the agent with a test cell, measuring a test level of nNOS activity in the test cell in the presence of the agent of interest, and determining the usefulness of the agent for the mitigation of one or more FXS. The determination is made by comparing the test level with a control level obtained from a control cell.

If the test agent or the combination of test agents is able to increase nNOS activity in the test cell (when compared to nNOS activity in the control cell), then the test agent or the combination of test agents is determined to be useful for the mitigation of one or more symptoms of FXS. If the test agent or the combination of test agents is not able to increase nNOS activity in the test cell (e.g., the test level is equal to or lower than the control level), then the test agent or the combination of test agents is determined not to be useful for the mitigation of one or more symptoms of FXS.

The term “test cell” as used herein refers to a brain cell that is capable of expressing neuronal nitric oxide synthase (nNOS). In some embodiments, the test cell is also capable of expressing endothelial nitric oxide synthase (eNOS) and/or NMDAR. In an embodiment, the brain cell is a neuron. Examples of neurons that are capable of expressing nNOS (and optionally NMDAR) include, but are not limited to molecular layer interneurons (stellate cells) and granule cells (GCs). Examples of brain cells that are capable of expressing eNOS (and optionally) include, but are not limited to, cells forming cerebral arteries (e.g., pericytes and/or endothelial cells). In some embodiments, the test cell and/or the control cell is derived from one or more individuals having FXS. In other embodiments, the test cell and/or the control cell is derived from one or more animal that is a model of FXS. In one set of embodiments, the test cell and/or the control cell is derived from an Fmr1 knock out mouse. The test cell and/or the control cell may be an in vitro or an ex vivo cell. The test cell and/or the control cell may be located within a brain sample (i.e. a test brain sample and/or a control brain sample) that is derived from an individual with FXS or from an animal model of FXS. In some embodiments, the test cell and/or the control cell is derived from the cerebellum of an individual with FXS or from an animal model of FXS. The test cell and/or the control cell may be located in vivo within a brain.

The term “control cell” as used herein may refer to a test cell before contacting the agent or the combination of agents. The control cell can also refer to a cell which is not placed in contact with the agent or the combination of agents and can instead be placed in contact with a control agent (an agent not capable of increasing nNOS activity such as, for example, a solution for diluting the test agent). In some embodiments, the control cell can be a brain cell that is capable of expressing nNOS, eNOS and/or NMDAR in the absence of the agent. The control cell may also be located in situ within a brain sample (ie. a control brain sample) that is derived from an individual with FXS, an animal model of FXS or a isogenic WT animal. In some embodiments, the control brain sample is derived from the same individual having FXS as the test cells. In some embodiments, the control brain sample is derived from the same animal model of FXS as the test cells. In one set of embodiments, the control cells are derived from an Fmr1 knock out mouse.

In the screening methods of the present disclosure, a test agent or a combination of test agents is contacted with the cell capable of expressing/expressing nNOS. The term “contacting” as used herein refers to putting the agent or the combination of agents of interest in physical contact with the cell or sample of interest by culturing, spraying, pouring, coating, rubbing or bathing the cell with the agent/combination of agents being screened.

The terms “test level of activity” or “test value” as used herein refers to a measurable phenotype that is associated with the activity of nNOS in the test cell or in the cells of a test brain sample. In some embodiments, the test level of activity being measured comprises the level of nNOS or eNOS activity in the test cell or in cells of the test brain sample. This can be obtained for example, by determining the amount of NO that is being produced in the presence of the test agent/the combination of test agents. This can further be obtained, for example, by determining the amount of cGMP produced by neighbouring smooth muscle cells in the test brain sample. This can also be obtained, for example, by determining the amount of the nNOS polypeptide and/or mRNA encoding the nNOS polypeptide in the test cell and/or test brain sample. In some embodiments, the test level of activity being measured comprises the level of NMDAR activity in the test cell or in cells of the test brain sample by determining for example, the level of Ca²⁺ transduced inside the test cell and/or the amount of NO being produced by the test cell. This can further be obtained, for example, by determining the amount of cGMP produced by neighbouring smooth muscle cells in the test brain sample. This can also be obtained, for example, by determining the amount of the NMDAR polypeptide and/or mRNA encoding the NMDAR polypeptide in the test cell and/or test brain sample.

The test level can further be obtained, for example, by determining if the test agent or the combination of test agents are capable of inhibiting the activity of a phosphodiesterase capable of hydrolyzing cGMP (and, optionally, also cAMP). In some embodiments, the method can include determining the test agent or the combination of test agents is capable of inhibiting at least one or more of PDE1, PDE2, PDE5 or PDE10. In some embodiments, the method can include determining the test agent or the combination of test agents is capable of inhibiting at least two or more of PDE1, PDE2, PDE5 or PDE10. In some embodiments, the method can include determining the test agent or the combination of test agents is capable of inhibiting at least three or more of PDE1, PDE2, PDE5 or PDE10. In some embodiments, the method can include determining the test agent or the combination of test agents is capable of inhibiting PDE1, PDE2, PDE5 and PDE10. In some embodiments, the method can screen for the usefulness of a test agent/combination of agents which is already known to inhibit a PDE capable of hydrolyzing cGMP.

The terms “control level of activity” or “control value” as used herein refers to a measurable phenotype that is associated with the activity of nNOS in the control cell or in the cells of a control brain sample. In some embodiments, the control level of activity being measured comprises the level of nNOS or eNOS activity in the control cell or in cells of the control brain sample. This can be obtained for example, by determining the amount of NO that is being produced in the control cell or the control brain sample. This can further be obtained, for example, by determining the amount of cGMP produced by neighbouring smooth muscle cells in the control brain sample. This can also be obtained, for example, by determining the amount of the nNOS polypeptide and/or mRNA encoding the nNOS polypeptide in the control cell and/or control brain sample. In some embodiments, the control level of activity being measured comprises the level of NMDAR activity in the control cell or in cells of the control brain sample by determining for example, the level of Ca²⁺ transduced inside the control cell and/or the amount of NO being by the control cell. This can further be obtained, for example, by determine the amount of cGMP produced by neighbouring smooth muscle cells in the control brain sample. This can also be obtained, for example, by determining the amount of the NMDAR polypeptide and/or mRNA encoding the NMDAR polypeptide in the control cell and/or control brain sample.

The control level can further be obtained, for example, by providing a control agent to the control cell, wherein the control agent lacks the ability of inhibiting the activity of a phosphodiesterase capable of hydrolyzing cGMP (and optionally cAMP).

In embodiments in which the method is practiced with a test brain sample, one or more test value concerning a measurement of the level of intrinsic plasticity via sodium channels, the degree of vasodilation, and/or the level of GABAergic inhibitory synaptic plasticity can be obtained from the test brain sample (and in some embodiments, from the control brain sample). The test agent or the combination of test agents can be considered useful if they increase in the test brain sample, when compared with the control brain sample, at least one of intrinsic plasticity via a sodium channel; a degree of vasodilation; or a level of GABAergic inhibitory synaptic plasticity. The test agent or the combination of test agents can be considered useful if they increase in the test brain sample, when compared with the control brain sample, at least two of intrinsic plasticity via a sodium channel; a degree of vasodilation; or a level of GABAergic inhibitory synaptic plasticity. The test agent or the combination of test agents can be considered useful if they increase in the test brain sample, when compared with the control brain sample, intrinsic plasticity via a sodium channel; a degree of vasodilation; and a level of GABAergic inhibitory synaptic plasticity. In embodiments in which the test agent cannot increase intrinsic plasticity via a sodium channel, a degree of vasodilation as well as a level of GABAergic inhibitory synaptic plasticity, the method can include combining such test agent with a further test agent to provide a combination of test agents to determine if the combination is able to increase intrinsic plasticity via a sodium channel, a degree of vasodilation as well as a level of GABAergic inhibitory synaptic plasticity. In some embodiments, the test agent or the combination of test agents is not considered useful if they fail to increase in the test brain sample, when compared with the control brain sample, at least one of intrinsic plasticity via a sodium channel; a degree of vasodilation; or a level of GABAergic inhibitory synaptic plasticity.

Measurements of the level of intrinsic plasticity via sodium channels can comprise determining the action current of cell-attached recordings in a test brain sample (and optionally in the control brain sample). Measurement of the degree of vasodilation can comprise determining the size (e.g., volume occupied by, level of vasoconstriction, level of vasodilatation, diameter, circumference, etc.) of cerebral blood vessels in a test brain sample (and optionally in the control brain sample). Measurements of the level of GABAergic synaptic plasticity can comprise current-clamp recordings of cells in test brain samples (and optionally of the control brain sample). Measurements of the level of GABAergic synaptic plasticity can comprise voltage-clamp recordings of cells in test brain samples (and optionally of the control brain sample).

### Example 1

It has been previously shown that Fragile X Syndrome (FXS) is characterized by deficits in a number of neurotransmitter systems, including signaling by glutamatergic and GABAergic neurotransmitter systems. In order to gain a better understanding of the FXS brain, it was tested whether glutamatergic signaling is intact in cerebellar stellate cells from Fmr1 KO mice, patch-clamping electrophysiology experiments were performed on acutely-isolated cerebellar brain slice tissue and stimulated parallel fibers (PFs) of cerebellar granule cells to evoke membrane current responses from stellate cell glutamatergic synapses of WT and Fmr1 KO mice.

### Methods

Animals: Wild-type mice with a C57BL/6J background were obtained from Charles River Laboratories (Wilmington, MA, USA) and maintained as a breeding colony at McGill University. Breeder pairs of Fmr1-KO mice and Gabra3 KO (1-Gabra3tm2Uru/Uru), C57BL/6 background, were kindly provided by Dr. Greenough (University of Illinois, Urbana-Champaign, IL 61801, USA) and Dr. Rudolph (Harvard Medical School, McLean Hospital, MA 02478, USA). Both male and female wild-type mice used for experiments ranged from postnatal days 21 to 35.

Cerebellum slice preparation: Mice (P21-35) were anesthetized with isoflurane and immediately decapitated. A block of cerebellar vermis was rapidly dissected from the mouse head and submerged in an ice-cold cutting solution perfused with carbogen gas (95% O₂, 5% CO₂). Cutting solution contains (in mM): 235 sucrose, 2.5 KCl, 1.25 NaH₂PO₄, 28 NaHCO₃, 0.5 CaCl₂, 7 MgCl₂, 28 D-glucose, 1 ascorbic acid, and 3 sodium pyruvate (pH 7.4; 305-315 mOsmol/L). The block of vermis was then fastened to a platform, transferred to the slicing chamber and again submerged in ice-cold cutting solution, bubbled with carbogen throughout the remainder of the procedure. Thin slices of cerebellar vermis (300 μm) were obtained with a vibrating tissue sectioner (Leica VT1200; Leica Instruments, Nussloch, Germany). The slices were transferred to oxygenated artificial cerebrospinal fluid (ACSF) and held at room temperature (21° C.-23° C.) for at least 1 h before recordings were performed. ACSF contained the following (in mM): 125 NaCl, 2.5 KCl, 1.25 NaH₂PO₄, 26 NaHCO₃, 2 CaCl₂), 1 MgCl₂, 25 D-glucose (pH of 7.4; 305-315 mOsmol/L).

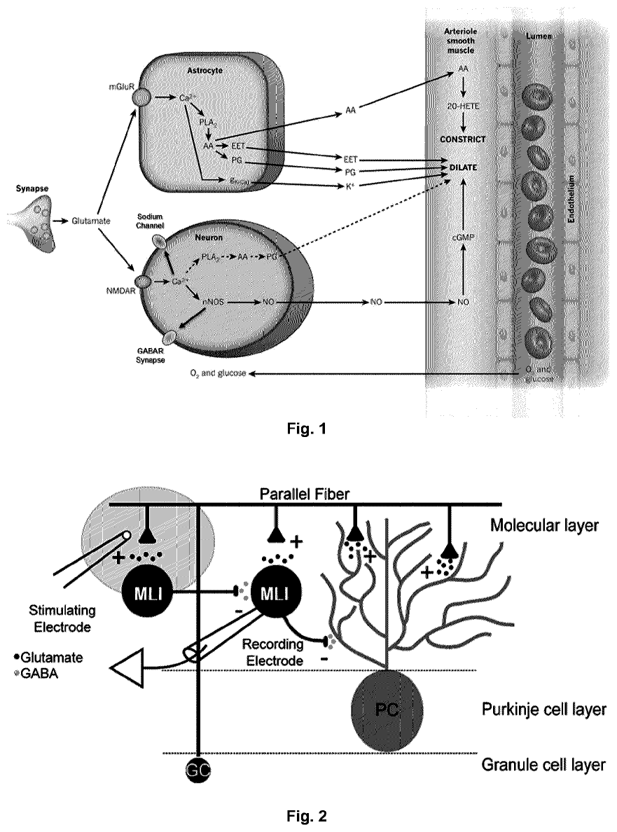

Electrophysiology and recording solutions: Whole-cell patch-clamp recordings were made from either visually-identified stellate cells in acute sagittal slices of cerebellar vermis using the arrangement of electrodes shown in FIG. 2. stellate cells were distinguished from misplaced or migrating granule cells by their small soma diameter (8-9 μm), location in the outer two-thirds of the molecular layer and whole-cell capacitance measurement (4-12 pF). Patch pipettes were prepared from thick-walled borosilicate glass and had open tip resistances of 4-7 MΩ when filled with an intracellular recording solution. Recordings were made with a Multiclamp 700A amplifier at a holding potential of −60 mV. Series resistance and whole-cell capacitance were estimated by cancelling the fast transients evoked at the onset and offset of a 10 ms, 5 mV voltage-command steps. Access resistance during whole cell recording (10-25 MΩ) was compensated between 60 and 80% and checked for stability throughout the experiments (˜15% tolerance). The bath was continuously perfused at room temperature (21-23° C.) with ACSF at a rate of 1-2 ml/min. Currents were filtered at 5 kHz with an eight-pole low-pass Bessel filter and digitized at 25 kHz with a Digidata 1322A data acquisition board and Clampex 10.1 software.

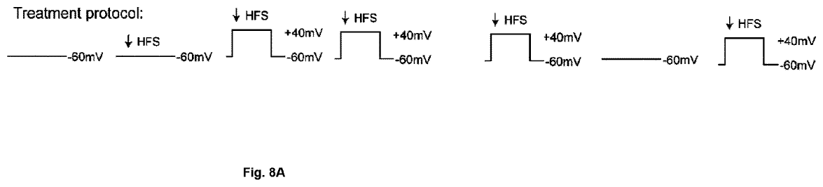

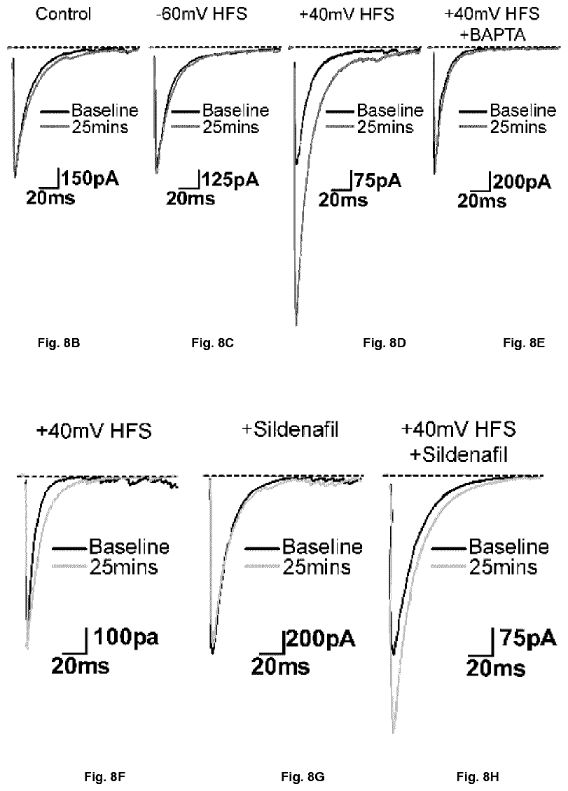

For extracellular stimulations, thin walled borosilicate glass electrodes (OD 1.65 mm, ID 1.15 mm; King Precision Glass Inc, Claremont, CA, USA) were used with a tip resistance of <3 MΩ when filled with ACSF. The ground electrode for the stimulation circuit was made with a platinum wire wrapped around the stimulation electrode. The stimulating electrode was positioned in the molecular layer at or just beneath the slice surface. Voltage pulses (10-25 V in amplitude, 200-400 μs in duration) were applied at low frequency stimulation (0.1 Hz) through the stimulating electrode. To minimize variability between responses, the stimulating electrode was positioned 50-100 μm away from the recorded cell. The stimulus voltage used during each experiment was at the lowest intensity to elicit the maximal eEPSP/IPSC response within the range described above. Stimulation strength and duration were kept constant throughout the experiment. For high frequency stimulation (HFS), trains of six stimuli were delivered at 100 Hz (inter-train interval of 20 s) as described previously (Larson et al, 2020). This HFS protocol has been used previously to potentiate inhibitory signaling through a ROS mediated pathway and mimics somatosensory stimulation patterns. The HFS protocol was performed every five minutes. During the voltage clamp experiments of evoked GABA currents (see FIGS. 8A-8I), the HFS protocol was performed at a holding potential of +40 mV to ensure relief of the Mg²⁺ block of NMDARs. The single stimulation recordings were performed at −60 mV to isolate the response from NMDA currents and GYKI 53655 was used to pharmacologically block AMPA currents. For all experiments which included perfusion of either pharmacological or peptide blocker compounds in the internal solution, the HFS induction protocol started after a 10-minute perfusion. Internal pipette solution for current-clamp experiments contained (in mM): 126 K-gluconate, 5 HEPES, 4 NaCl, 15 D-glucose, 0.05 CaCl₂, 1 MgSO₄, 0.15 K4-BAPTA, 3 Mg-ATP, 0.1 Na-GTP, 2 QX314 (adjusted to pH 7.4 with KOH, 300-310 mOsmol/L). Voltage-clamp recordings were made with an intracellular solution that contained (in mM): 140 CsCl, 4 NaCl, 0.5 CaCl₂), 10 HEPES, 5 EGTA, 2 Mg-ATP, 2 QX314 (pH 7.4 with CsOH, 300-310 mOsmol/L). For cell-attached experiments, internal solution contained (in mM): 125 NaCl, 10 HEPES, 40 D-Glucose, 2.5 MgCl₂ (adjusted to pH 7.4 with NaOH, 300-310 mOsmol/L).

Pharmacological compounds: NMDAR antagonist, APV (10 μM) and MK-801 (10 μM), AMPA receptor antagonist 1-(4-Aminophenyl)-3-methylcarbamyl-4-methyl-3,4-dihydro-7,8-methylenedioxy-5H-2,3-benzodiazepine hydrochloride (GYKI 53655; 10 μM), and the GABA-A receptor antagonist bicuculline (10 μM) were purchased from Tocris Bioscience (Ellisville, MO, USA). Stock solutions of these antagonists were prepared in water and were stored at −20° C. and working solutions were diluted with ACSF shortly before application to the bath. Phorbol 12-myristate 13-acetate (PMA, 100 nM. Tocris) was dissolved in DMSO and stored at −20° C. The final maximum DMSO concentration for all experiments (0.1% v/v).

Vascular reactivity: Middle or posterior cerebral arteries were isolated from both WT (n=9) and Fmr1-KO (n=4) mice and vessel diameter was measured using video microscopy (Living Systems Instrumental, Burlington, VT) as previously described. Blood vessels were cannulated in a closed sac preparation in 1×KREBS buffer, and gradually pressurized to 60 mmHg. Vasoconstriction was measured in response to extraluminal application of either thromboxane A2 receptor agonist U46619, NMDA, or acetylcholine (ACh) at increasing concentrations (10-9 to 10-3 mol/L, Tocris Bioscience Ellisville, MO, USA). Data is presented as a percentage change from the basal diameter.

Acute slice vascular reactivity: Slices of cerebellar vermis were prepared as described above (see Cerebellum Slice Preparation). Imaging experiments were performed on an Olympus BX51 upright microscope (Olympus, Southall, UK) equipped with infrared optics. Slices were continually perfused with oxygenated ACSF. Blood vessels were visually identified in the molecular layer and images were taken at 4 Hz with an Olympus XM10 camera. Baseline recordings were then conducted for 5 minutes to ensure stability which was followed by perfusion of the thromboxane A2 receptor agonist U46619 (75/150 nM) to saturation within 10 minutes. Upon saturation of U46619 (10 min), NMDA (50 μM) was washed into the slice chamber for 5 minutes while U46619 concentrations were maintained. Imaging then continued for 15 minutes after NMDA washout while slices were again perfused with U46619-containing ACSF. Blood vessel diameters were analyzed using a custom Matlab script kindly provided by Drs. Bruno Cauli (Sorbonne Université, France) and Elizabeth Hillman (Columbia University, USA).

Behavior experiments-prepulse inhibition: PPI of the acoustic startle response (ASR) was studied in post-pubertal (PD 56-90) animals using an SR-LAB system (San Diego Instruments, San Diego, CA, USA) comprising two sound-attenuating chambers, each equipped with a cylindrical Plexiglas animal enclosure (length 16 cm, inner diameter 8.2 cm). A speaker positioned 24 cm directly above the enclosure provided the broadband tone pulses. A piezoelectric accelerometer affixed to the animal enclosure frame was used to detect and transduce motion resulting from the animals' startle response. Tone pulse parameters were controlled by a microcomputer using the software package (SR-LAB) and interface assembly that also digitized (0-4095), rectified, and recorded stabilimeter readings.

All PPI studies were conducted between 09:00 and 17:00 h. Animals randomly received either saline or sildenafil (7.5 mg/kg i.p.) treatment. After a 10-min waiting period following treatment, saline-treated and drug-treated animals were placed in the Plexiglas enclosure and allowed to acclimatize to the environment with background noise of 70 dB for 5 min before being tested during 32 discrete trials. On the first two trials, the magnitude of the startle response to a 30 ms lasting 120 dB tone was measured. These first two startle tones were presented to habituate the animals to the testing procedure and thus were omitted from the data analysis. On the subsequent 30 trials, the startle tone was either presented alone or 100 ms after presentation of prepulses of 30 ms duration with intensities ranging from 6 dB to 15 dB above background noise (i.e. 76-85 dB) that varied randomly between the trials. ASR was measured at each of the four prepulse intensities on five trials; animals were randomly presented with the startle tone alone during the other 10 trials. The same stimulus condition was never presented on more than two consecutive trials. The interval between each trial was programmed to a variable time schedule with an average duration of 15 s (range 5-30 s). A measure of ASR amplitude was derived from the mean of 100 digitized data-points collected from stimulus onset at a rate of 1 KHz.

Behavior experiments-locomotor activity: The locomotor activity was measured in an environment (activity boxes) novel to the animals, as described previously. Briefly, mice were handled for about 5 minutes once a day for one week before the testing. On the day of testing, animals were brought in their home cages to the anteroom (a room adjacent to the testing room separated by a door) and kept there for 30 minutes before drug or vehicle administration. Animals of both genotypes were randomly divided into two groups. One group received an i.p. injection of sildenafil (7.5 mg/kg; drug dissolved in sterilized PBS with 2% DMSO) and the other group an i.p. injection of the vehicle. One hour after the drug or vehicle administrations, the animals were placed in individual activity boxes (AccuScan Instruments, Inc., Columbus, OH, USA) (L×W×H=17.5 cm×10 cm×26 cm) in a dimly lit testing room where their locomotor activity was monitored for 90 min. The activity boxes were equipped with infrared sensors; beam breaks by the animals were used to assess locomotor activity. Data were collected using the Versamax™ Software (version 4.0, 2004; AccuScan Instruments, Inc.). The total horizontal activity for the whole 90 min session was used in the analysis.

### Stellates Display Reduced NMDAR Responses in a Mouse Model of Fragile X Syndrome.

Stimulation of parallel fibers (PFs) in wild-type and Fmr1 KO cerebellar brain slices with a single stimulus was sufficient to activate synaptic AMPARs in both WT and Fmr1 KO stellate cells (FIGS. 3A-3B). In fact, a direct comparison of the amplitude and decay kinetics of the AMPAR response showed that these responses were indistinguishable in stellate cells from WT and Fmr1 KO mice (FIG. 3C). In contrast, when WT and Fmr1 KO brain slices were treated with 10 μM of GYKI 53655 (or GYKI for short) (to block AMPA currents) and PFs were subjected to high frequency stimulation, robust extrasynaptic NMDAR response was induced in WT stellate cells with a much weaker response in stellate cells from Fmr1 KO mice (FIGS. 3D-3E).

### Loss of Neuronal Firing in Fmr1 KO Mice Due to NMDAR Hypofunction

High frequency stimulation of PFs has been shown to induce a NMDAR-dependent intrinsic plasticity of Na⁺-channels that augments action potential firing rates for extended periods in cerebellar stellate cells. In order to assess the consequences of NMDAR hypofunction on the intrinsic excitability of stellate cells, action currents in cell-attached recordings from stellate cells isolated from WT and Fmr1 KO mice were measured after high frequency stimulation of PFs, in the presence of absence of the NMDAR antagonist APV (FIGS. 4A-4I). High frequency stimulation of PFs induced a long-term 2-fold increase in the basal firing rates of WT cerebellar stellate cells (FIGS. 4J and, 4M). In contrast, no long-term increase in basal firing rates was observed in WT stellate cells treated with APV or in stellate cells from Fmr1 KO (FIGS. 4K, 4L and 4M). Notably no significant difference was observed between the basal firing properties of the stellate cells from WT and Fmr1 KO mice (FIG. 4N). Together, this data indicates that the loss of intrinsic plasticity observed in Fmr1 KO mice is entirely explained by the significant deficit in the magnitude of their NMDAR responses.

### Loss of Vascular Reactivity in Fmr1 KO Mice Due to NMDAR Hypofunction

It has been previously shown that NMDARs expressed by stellate cells promote vasodilation of local cerebellar blood vessels by generating nitric oxide which stimulates guanylate cyclase to elevate cGMP. Therefore, the consequences of NMDAR hypofunction on vasodilation were investigated. Briefly, cerebellar brain slices from WT and Fmr1 KO mice were exposed to 75 nM of the thromboxane A2 agonist U46619, in order to induce vasoconstriction, and were subsequently subjected to a bath application of 50 μM NMDA for 5 mins.

Unexpectedly, constriction of blood vessels in the Fmr1 KO brain slices in response to treatment with 75 nM U46619 occurred much less frequently than it did in the WT brain slices (FIGS. 5A-5F and Table 1). While the frequency of vasoconstriction increased in both treatment groups, when the concentration of U44619 was increased to 150 nM, the Fmr1 KO brain slices still exhibited reduced vasoconstriction compared to WT (Table 1 and FIG. 5H). Separate vascular reactivity experiments were also carried out using middle (MCA) or posterior (PCA) cerebral arteries from WT and Fmr1 KO mice. Even under these conditions, vasoconstriction in response to various concentrations of U46619 was reduced in Fmr1 KO blood vessels compared to WT. This revealed an apparent dysfunction in thromboxane A2 receptor signaling in the Fmr1 KO mice.

TABLE 1
Summary of the proportion of blood vessels from WT and Fmr1 KO
mice that constricted in response to treatment with 75 nm or 150 nm
of U46619 compared to the proportion that did not respond

| | | [U46619] 75 nM | [U46619] 150 nM |
|---|---|---|---|
| WT | No response | 3 | 2 |
| | Response | 10 (77%) | 18 (90%) |
| Fmr1 KO | No response | 2 | 11 |
| | Response | 8 (80%) | 25 (69%) |

Subsequent bath application of NMDA (50 μM, 5 mins) to the WT brain slices caused vasodilation of blood vessels in close vicinity of stellate cells (FIGS. 5A-5F and 5H). The degree of vasodilation observed in the WT brain slices was proportional to the vasoconstriction elicited by 75 nM U46619 (FIG. 5I). Although U44619 was able to induce vasoconstriction in some blood vessels from Fmr1 mice (Table 1 and FIG. 5G), particularly when administered at a higher concentration, subsequent bath application of NMDA failed to promote blood vessel dilation (FIGS. 5H and 5I).

To better understand the defects in both vasoconstriction and vasodilation, a comparison of the properties of blood vessels in both the cerebellum and somatosensory cortex from wildtype (WT) and Fmr1 KO mice was performed (FIGS. 5J-5O). Measurement of the resting diameter of all blood vessels reveals that almost all the responses observed correspond to that of capillaries (FIG. 5J) and not arterioles or arteries which have a larger diameter. Although the measurements reveal a range of diameters, the mean value was close to 7 microns which corresponds to the diameter of capillary blood vessels in the mouse brain. Accordingly, it was concluded that the responsiveness of the blood vessels described in the experiments are primarily due to capillaries. Capillaries lack smooth muscle and therefore vascular reactivity is mediated primarily by pericytes. In WT tissue, bath application of the neurotoxin, tetrodotoxin (1 mM TTX), prior to the application of NMDA (50 μM, 5 mins) completely blocked vasodilation demonstrating that NMDA induces its effect by an action through neurons, and not astrocytes (FIG. 5K). As anticipated, bath application of NMDA failed to induce vasodilation in capillaries from both the cerebellum and cortex of Fmr1 KO mice (FIGS. 5L-5M). In each case, the robust vasodilation elicited by NMDA in both wildtype cerebellar and cortical blood vessels was lost in blood vessels taken from Fmr1 KO mice. Pre-incubation with sildenafil alone had little effect on the NMDA-induced vasodilation in the cerebellum but elicited vasodilation in some blood vessels. However, bath application of sildenafil with the antioxidant, N-acetylcysteine (NAC), almost completely recovered the NMDA induced vasodilation in blood vessels from fmr1 KO to levels seen in wild type tissue. Note that blood vessels in the cerebellum and context from Fmr1 KO mice responded to papaverine which has a direct action on the blood vessel. The positive responsiveness to papaverine reveals that the absence of vasodilation to NMDA in tissue from Fmr1 KO mice is not due to a defect in the blood vessel tissue. This finding demonstrates that the defect in neurovascular coupling is not unique to a single brain region but is likely pervasive throughout the mammalian CNS. Interestingly, pre-incubation with sildenafil (100 mM) had little or no effect on the NMDA response in blood vessels from the Fmr1 KO cerebellum but was able to rescue some of the deficits in, at least, some of blood vessels from the Fmr1 KO cortex (FIGS. 5L-5M). This observation suggests that there may be some biological differences in the two brain regions in their responsiveness to NMDA. Most importantly, co-application of sildenafil with the antioxidant. N-acetylcysteine (1 mM NAC) almost completely restored the ability of NMDA to induce vasodilation in blood vessels from Fmr1 KO mice (FIGS. 5L-5M). This finding reveals that tissue from fmr1 KO mice must have elevated levels of reactive oxygen species which can be mitigated by the addition of NAC. Accordingly, NAC or molecules with similar antioxidant properties should be considered as an additional therapeutic in the treatment of FXS. As a control, the actions of papaverine (100 mM) which has a direct action on blood vessels were tested. In both cases, papaverine was able to induce vasodilation in blood vessels from the cerebellum and cortex of Fmr1 KO mice (FIGS. 5L-5M) revealing that the capillary itself is not defective. Finally, the ability of U44619 to vasoconstrict blood vessels in WT and Fmr1 KO in all the conditions was similar (FIGS. 5N-5O) demonstrating that the inability of NMDA to induce vasodilation in Fmr1 KO mice is not due to variations in the effectiveness of U44619. Note that the degree of vasodilation was similar under all conditions demonstrating that the differences in vasodilation observed in FIGS. 5K-5M cannot be due to varying effects of the thromboxane A2 vasoconstrictor, U44619.

### NMDAR Signaling Deficits in Fmr1 KO Mice Reduces GABAR Plasticity

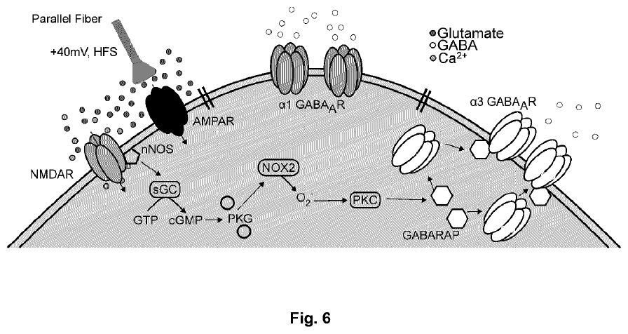

It was previously shown, from work involving HFS of PFs to activate extrasynaptic NMDARs, that NMDARs strengthen α3-containing GABAR synapses through a Ca²⁺/nNOS dependent pathway (summarized in FIG. 6). To determine whether NMDAR signaling deficits in Fmr1 KO mice impact nitride oxide (NO) signaling to GABAR synapses, separate current-clamp (FIGS. 7A-7E) and voltage-clamp (FIGS. 8A-8I) experiments were carried out.

For the current-clamp experiments, PFs in WT cerebellar brain slices and Fmr1 KO cerebellar brain slices were subject to high frequency stimulation. Current-clamp recordings of PF-evoked synaptic events induced a biphasic or dual synaptic response consisting of an initial depolarizing excitatory postsynaptic potential (EPSP) followed by a slower hyperpolarizing inhibitory postsynaptic potentials (IPSP) in both treatment groups (FIGS. 7A-7B). Additionally, in WT mice, a time dependent reduction in the EPSP amplitude was observed following HFS of PFs due to the strengthening of GABAR synapses (FIGS. 7A-7D). Strikingly, no time dependent reduction in the EPSP amplitude was observed following HFS of PFs from Fmr1 KO mice, suggesting that the strengthening of GABAR synapses did not occur in this group (FIGS. 7B, 7D. and 7E).

For the voltage-clamp experiments, wild-type stellate cells displayed a 2-fold increase in the peak amplitude of pharmacologically-isolated GABAR currents after 25 minutes, compared to the baseline, when the HFS protocol was paired with depolarization (FIGS. 8A-8E). No such increase in the peak amplitude of pharmaceutically-isolated GABAR currents was observed in Fmr1 KO stellate cells under the same conditions, which demonstrates that GABAR plasticity is absent in Fmr1 KO mice (FIGS. 8F-8I).

### The PDE5 Inhibitor Sildenafil can Rescue the Neuronal and Behavioural Deficits Identified in Fmr1 KO Mice

A series of experiments were conducted to determine if sildenafil could restore the deficits in NMDAR-mediated inhibitory synapse strengthening and intrinsic plasticity observed in Fmr1 KO mice.

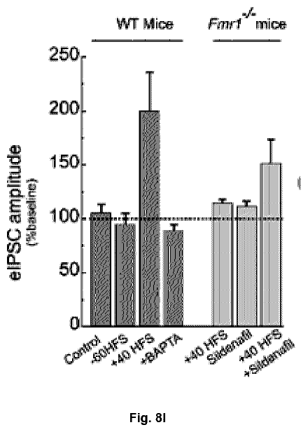

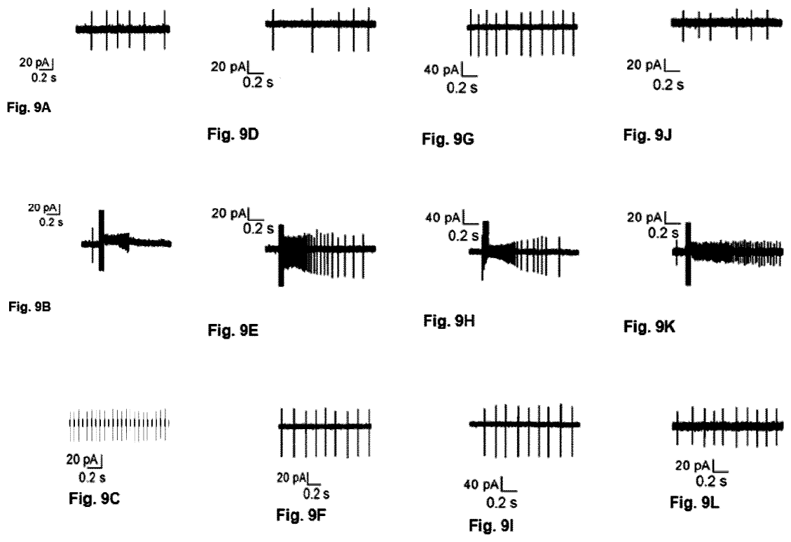

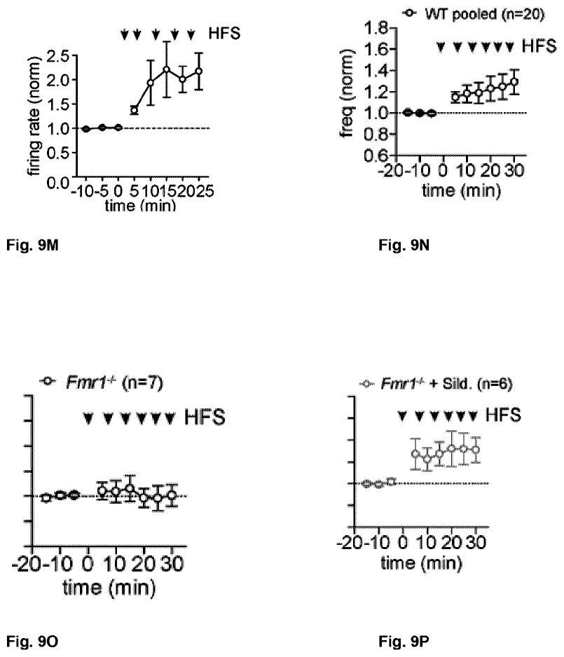

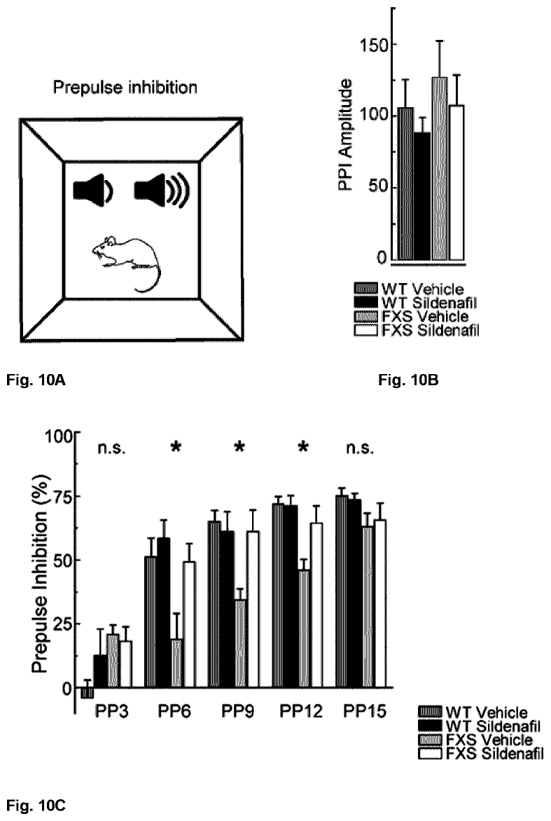

The ability of sildenafil to restore NMDAR-mediated inhibitory synapse strengthening in Fmr1 KO mice was investigated using current-clamp and voltage-clamp experiments. In both sets of experiments, treatment of cerebellar brain slices with 100 μM sildenafil rescued the complete loss of GABAR plasticity observed current-clamp and voltage-clamp recordings of Fmr1 KO stellate cells (FIGS. 7A-7E, 8F-8I). Sildenafil did not, however, affect baseline properties of these cells (data not shown). Likewise, pre-treatment with 100 μM sildenafil restored the long-term increase in baseline firing rates seen in WT stellate cells following HFS of PFs (FIGS. 9A-9P). Together, these observations demonstrated that sildenafil was able to rescue neuronal deficits identified in Fmr1 KO mice.

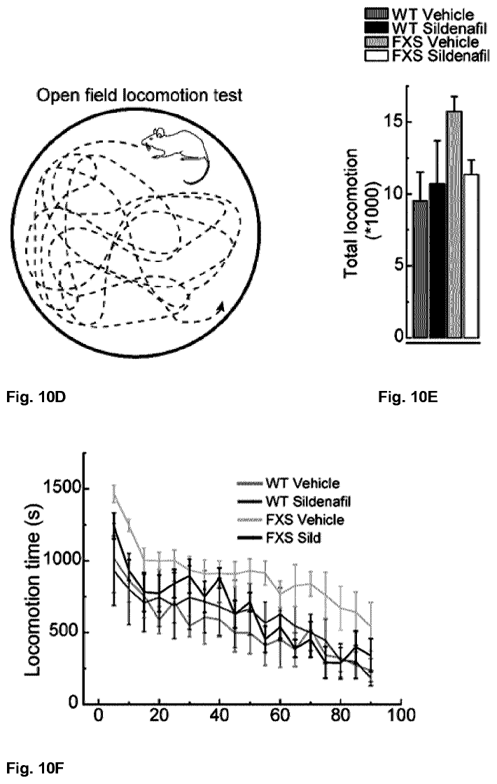

Certain behavior deficits are commonly observed in FXS patients and have also been described in Fmr1 KO mice and nNOS KO mice including: reduced prepulse inhibition, increased hyperactivity and increased anxiety (Table 2). In the next series of experiments, the ability of sildenafil to rescue some of these behavioral deficits in Fmr1 KO mice was investigated. Administration of a single dose of sildenafil (7.5 mg/kg) to Fmr1 KO mice was able to fully correct deficits in prepulse inhibition (PPI), a sensorimotor gating deficit found in FXS patients and in nitric oxide synthase 1 (NOS1) knockout mice (FIGS. 10A-10C). Administration of sildenafil also normalized the enhanced locomotor activity of Fmr1 KO mice, which is a putative correlate of the hyperactivity and anxiety seen in FXS patients (FIGS. 10D-10F). Taken together, this data suggests that the FXS brain exhibits hypofunction in nitric oxide signaling and that this defect may be treated by sildenafil and/or its analogs.

TABLE 2
Different behavioral deficits commonly exhibited by human
FXS patients and that are also frequently encountered in
Fmr1 KO mice and nNOS KO mice. Downwards-facing arrows
represent behaviors that are decreased in the FXS patients or
the Fmr1 and nNOS KO mice compared to control subjects.
Conversely, upwards-facing arrows represent those behaviors
that are increased in these groups compared to control.

| | Fmr1 | nNOS | |
|---|---|---|---|
| | KO | KO | FXS |
| BEHAVIORAL TEST | MICE | MICE | PATIENTS |
| PREPULSE INHIBITION | ↓ | Not | ↓ |
| | | determined | |
| HYPERACTIVITY | ↑ | ↑ | ↑ |
| CUED & CONTEXTUAL | ↓ | ↓ | ↓ |
| FEAR CONDITIONING | | | |
| STARTLE RESPONSE | ↓↑ | Not | ↑ |
| | | determined | |
| SOCIAL INTERACTION | ↓ | ↓ | ↓ |
| AGGRESSION | ↑ | ↑ | ↑ |
| ANXIETY | ↓↑ | ↑ | ↑ |
| OPEN FIELD (ANXIETY) | ↑ | ↑ | Not |
| | | | determined |
| REPETITIVE BEHAVIOR | ↑ | Not | ↑ |
| | | determined | |
| REVERSAL LEARNING | ↓ | ↓ | ↓ |
| SENSORY SENSITIVITY | ↑ | Not | ↑ |
| | | determined | |
| SPATIAL MEMORY | ↓ | ↓ | ↓ |

PDE1a & b. PDE 2a, PDE 5a and PDE 10a is Expressed in nNOS+ Neurons in Different Part of the Brain of WT Mice

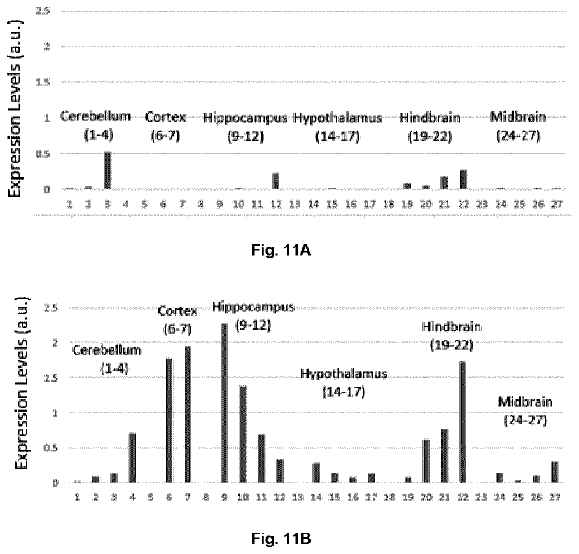

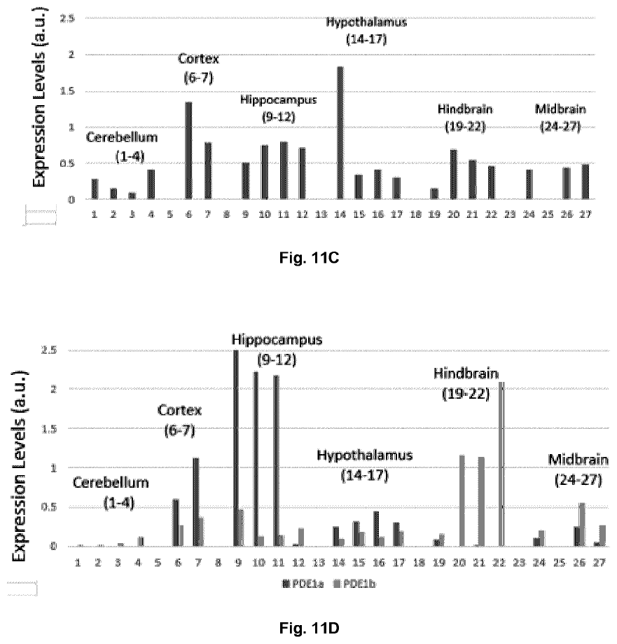

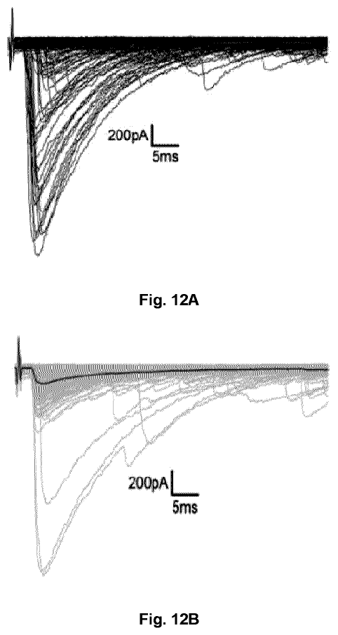

Using a published single-cell transcriptome atlas of WT mouse brain development (Zeisel et al., 2018 and mousebrain.org), the RNA sequencing data derived from nNOS+ neurons, from various parts of the brain, was obtained and the transcript levels corresponding to eleven PDE isoforms (PDE1-PDE11) in these neurons were analyzed (FIGS. 11A-11D). This data revealed that nNOS+ neurons localized in various regions of the brain of WT mice express PDE1a, PDE1b, PDE2a, and PDE10a, in addition to PDE5a. Transcripts corresponding to the other PDE isoforms were either expressed at significantly lower levels or were not detected at all in the nNOS+ neurons in the brains of WT mice. This suggests that inhibitors of PDE1, 2 and 10 may also be viable candidate molecules to be tested for their potential in treating symptoms related to FXS.

### Example 2

### Strengthening of Inhibitory Synapses is GABA Receptor Subunit Dependent

Immunohistochemical and targeted gene deletion studies have both demonstrated that α1-containing GABA receptors (GABARS) are the most abundant inhibitory neurotransmitter receptor in the mammalian brain. In contrast, α3-containing GABARs are thought to only play a minor role that diminishes throughout development. Under basal conditions, inhibitory synapses of stellate cells predominantly express α1-containing GABARs with lower contribution of α3-containing receptors. Interestingly, α3-containing GABARs play an important role but only following periods of sustained patterned activity that recruit α3-containing GABARs into inhibitory synapses. The mechanism of synapse strengthening is reliant on an increase in cytosolic reactive oxygen species (ROS). Interestingly, elevation in cytosolic ROS does not affect synapses containing α1 GABARs demonstrating that the plasticity mechanism is subunit specific. The inhibitory synapse strengthening of cerebellar granule cells is similarly subunit dependent. Like stellate cells, α1-containing GABAR synapses are the most dominant in basal conditions, however, ROS-dependent synapse strengthening is mediated by α6-containing GABARs. It was observed herein that cytosolic ROS can be elevated following activation of extrasynaptic NMDA-type ionotropic glutamate receptors (NMDARs). Extrasynaptic NMDARs are stimulated by the excitatory neurotransmitter, L-glutamic acid (L-Glu), which is released from presynaptic glutamatergic terminals of axons from cerebellar granule cells which together form parallel fibers (PF) (FIG. 6). Activation of NMDARs promotes the entry of extracellular Ca²⁺ which, in turn, stimulates the enzyme, neuronal nitric oxide synthase (nNOS), which catalyzes the synthesis of the gas, nitric oxide (NO), from the amino acid, arginine. The elevation in NO activates soluble guanylate cyclase (cGC) generates the second messenger signaling molecule, cyclic GMP (GMP), and triggers a cascade of signaling events starting with the sequential activation of protein kinase G (PKG), followed by NOX2 which elevates cytosolic ROS which, in turn, stimulates the activity of protein kinase C.

Through a mechanism not fully understood, the activation of PKC promotes the insertion of α3-containing GABARs into inhibitory synapses or inhibitory long-term potentiation (iLTP) through the scaffolding protein, GABARAP. This is in contrast to previous models that have been proposed for the strengthening of inhibitory synapses whereby iLTP reflects the accumulation of more GABARs into the same inhibitory synapses through the scaffolding protein, gephyrin. Taken together, the data of Examples 1 and 2 establish that under basal conditions, most inhibitory synapses contain α1-GABARs but that following activity dependent strengthening driven by NMDARs, α3-containing GABARs are recruited into synapses but to sites devoid of α1-GABARs (FIG. 6). The present disclosure therefore shows that stellate cells have “silent” GABAR synapses under basal conditions that become populated with α3-containing GABARs once the pathways that trigger recruitment are stimulated.

FIG. 6 schematically summarizes the main signaling events and molecules that lead to the selective recruitment of α3-containing GABARs into inhibitory synapses of cerebellar stellate cells. High frequency stimulation (HFS) of parallel fibers from granule cells stimulates extrasynaptic NMDARs of stellate cells activating nNOS through the influx of external Ca²⁺, nNOS generates NO, which acts on guanylate cyclase (sGC) to elevate cGMP which, in turn, stimulates PKG and NOX2. Without wishing to be bound by theory, the production of superoxide by NOX2 leads to the activation of PKC which then leads to the recruitment of GABARs via a GABARAP-dependent pathway. This signaling pathway selectively acts on α3-containing GABAARs and does not affect synapses containing α1-GABAARs.

### Methods

Animals: Wild-type mice with a C57BL/6J background were obtained from Jackson Laboratories (Bar Harbor, ME, USA) and maintained as a breeding colony at McGill University. Mice (male and female) used for the experiments ranged from 20 to 30 days old (P15-30). All experiments have been approved by the local authorities and were performed in accordance with the guidelines of the Canadian Council on Animal Care and were approved by the Animal Care Committee of McGill University. Breeder pairs of Fmr1 KO mice (C57BL/6 background) and Gabra3 KO (1-Gabra3tm2Uru/Uru), were kindly provided by Drs. Greenough (University of Illinois, Urbana-Champaign, IL 61801, USA) and Rudolph (Harvard Medical School, McLean Hospital, MA 02478, USA), respectively.

Cerebellar slice preparation: Mice were anaesthetized with isoflurane and immediately decapitated. The cerebellum was rapidly removed from the whole brain while submerged in oxygenated (95% O₂, 5% CO₂) ice-cold cutting solution (4° C.). Cutting solution contained (in mM): 235 sucrose, 2.5 KCl, 1.25 NaH₂PO₄, 28 NaHCO₃, 0.5 CaCl₂), 7 MgSO₄, 28 D-Glucose (pH of 7.4; 300-310 mOsmol/L). The tissue was maintained in ice-cold solution whilst sagittal slices of cerebellum (300 μm) were cut using a vibrating tissue slicer (Leica VT1200, Leica Instruments, Nussloch, Germany). The slices were transferred to oxygenated, room temperature (21-23° C.) artificial cerebrospinal fluid (aCSF) for at least 1 hr before recordings. aCSF contained (in mM): 125 NaCl, 2.5 KCl, 1.25 NaHaPO₄, 26 NaHCO₃, 2 CaCl₂), 1 MgSO₄, 25 D-Glucose (pH of 7.4; 300-310 mOsmol/L).

Electrophysiology: Slice experiments were performed on an Olympus BX51WI upright microscope (Olympus, Southall, UK) equipped with differential interference contrast/infrared optics. Whole-cell patch clamp recordings were made from cerebellar stellate cells. Stellate cells were distinguished from misplaced or migrating granule, glial, or basket cells by their small soma diameter (8-9 μm) and location in the outer two-thirds of the molecular layer. Voltage clamp recordings were made with patch pipettes prepared as described above but filled with an intracellular solution that contained (in mM): 140 CsCl, 4 NaCl, 0.5 CaCl₂), 10 HEPES, 5 EGTA, 2 Mg-ATP, 2 QX314 to block voltage-activated Na⁺ channels and 0.5 mg/ml 1 Lucifer Yellow as a post hoc dye indicator (pH 7.4 with CsOH, 300-310 mOsmol/L). Patch pipettes were prepared from thick-walled borosilicate glass (GC150F-10, OD 1.5 mm, ID 0.86 mm; Harvard Apparatus Ltd, Kent, UK) and had open tip resistances of 6-10 MΩ. Recordings were made with a Multiclamp 700B amplifier (Molecular Devices, Sunnyvale, CA, USA) at a holding potential of −60/−70 mV. Series resistance and whole-cell capacitance were estimated by cancelling the fast-current transients evoked at the onset and offset of brief (10 ms) 5 mV voltage-command steps. Series resistance during postsynaptic whole-cell recording (10-35 MΩ) was checked for stability throughout the experiments (<20% tolerance). The capacitance of the stellate cells was in the range of 5-14 pF. The bath was continuously perfused at room temperature (22-23° C.) with well-oxygenated aCSF at a rate of 1-2 mL/min. Currents were filtered at 5 kHz using an eight-pole low-pass Bessel filter (Frequency Devices, Haverhill, MA, USA) and digitized at 25 kHz with a Digidata 1322A data acquisition board and Clampex10 (Molecular Devices) software. Curve fitting and figure preparation of all electrophysiology data was performed using Origin 7.0 and Origin 2020 (OriginLab, Northampton, MA, USA), Microsoft Excel, and Clampfit 10 (Molecular Devices) software.

For extracellular stimulation, thin walled borosilicate glass electrodes (OD 1.65 mm, ID 1.15 mm; King Precision Glass Inc, Claremont, CA, USA) were used with a tip current of <3 MΩ when filled with aCSF. The ground electrode for the stimulation circuit was made with a platinum wire wrapped around the stimulation electrode. The stimulating electrode was positioned in the molecular layer at or just beneath the slice surface. Voltage pulses (1-5 V in amplitude, 200-400 μs in duration) were applied at low frequency stimulation (0.5 Hz) through the stimulating electrode. To minimize variability between responses, the stimulating electrode was positioned 50-100 μm away from the recorded cell. The stimulation intensity for minimal stimulation experiments was determined to be the minimal voltage to record a measurable eIPSC during 25-50% of the sweeps. Stimulation strength and duration were kept constant throughout the experiment. For high frequency stimulation (HFS), trains of six stimuli were delivered at 100 Hz (inter-train interval of 20 s) at the lowest intensity to elicit the maximal response (15-25V in amplitude). The HFS protocol has been previously shown to generate ROS and mimics somatosensory stimulation patterns. The HFS was performed every five minutes to ensure a continual accumulation of ROS. During the voltage clamp experiments of evoked GABA currents, the HFS protocol was performed at a holding potential of +40 mV to relieve Mg²⁺ of NMDAR. The single stimulation recordings were performed at −60 mV to isolate the response from NMDA currents and used NBQX to pharmacologically block AMPA currents.

Evoked inhibitory postsynaptic currents (eIPSCs) were recorded utilizing a low voltage stimulus (1-5 volts (V)). The frequency of stimulation was 0.5 Hz, indicating that a stimulus was given every 2 seconds, for a duration of 5 minutes, demonstrating 150 stimuli per recording. This was then followed by a HFS stimulation in which the cell was depolarized to +40 mv coupled with a high stimulus intensity of 15-20V for one minute. This was then followed by the MS paradigm again and repeated four times for a total duration of 25-30 minutes. This allowed to accurately assess the synaptic connectivity within a single cell over time from baseline to 25 minutes after the HFS protocol was induced.

### Minimal Stimulation Protocol Undercovers the Existence of Silent Inhibitory Synapses

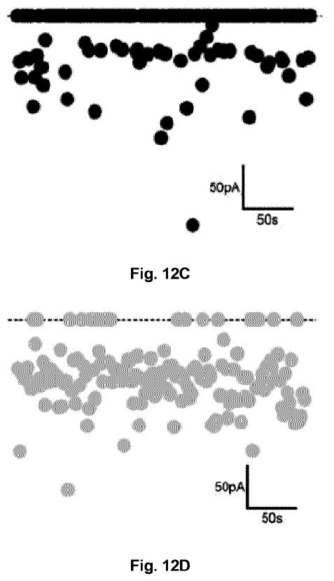

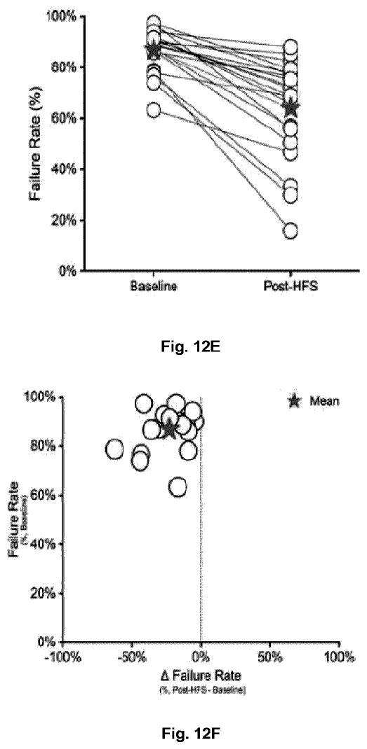

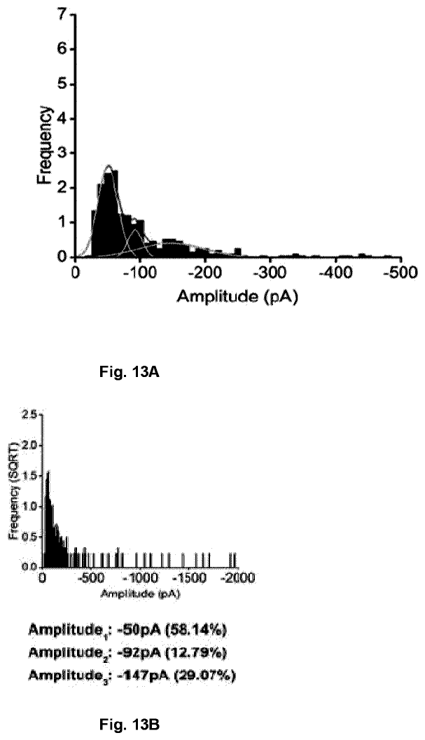

A stimulation protocol was designed to further test for the existence of silent GABAR synapses. By showing an increase in connectivity at inhibitory GABAergic synapses following activity dependent stimulation of NMDARs, the increase would provide evidence for the formation of new synapses. To do this, PFs were first stimulated in baseline conditions with a reduced voltage stimulus so that the probability of GABA release is low (FIGS. 12A and 12C). Using this minimal stimulation protocol, a high failure rate of GABAergic transmission was observed corresponding to 86.9+/−0.02% (Mean±s.e.m, n=20) of the time (FIG. 12E).

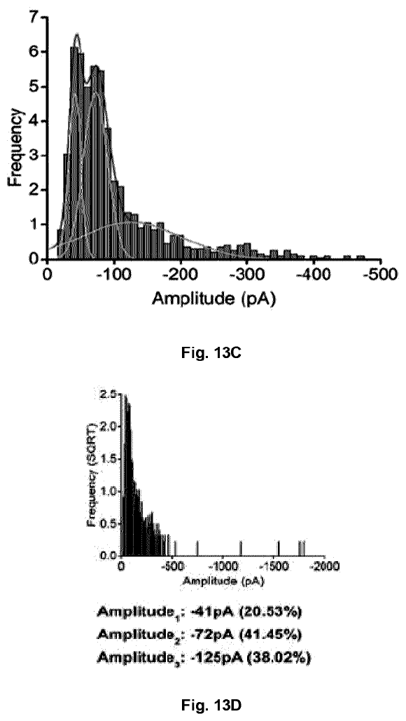

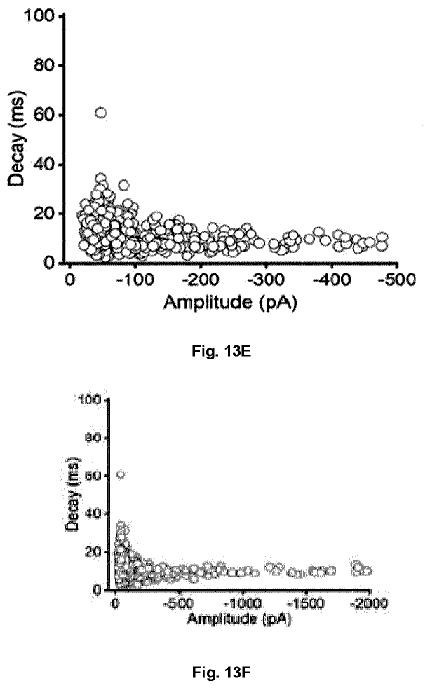

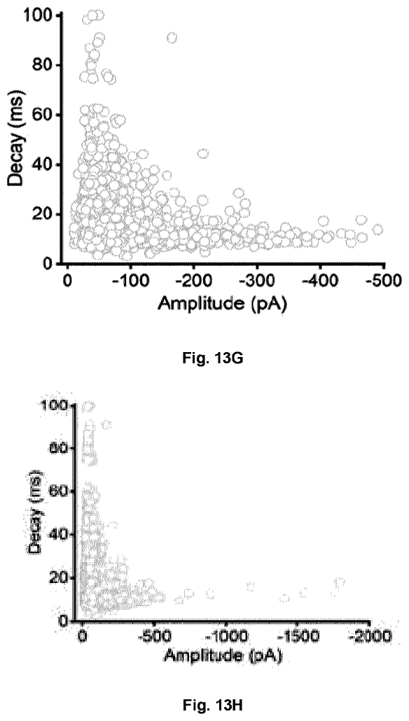

All synaptic events from every cell were plotted on a frequency histogram for both baseline (FIGS. 13A-13B) and post-HFS (FIGS. 13C-13D). FIGS. 13A and 13C illustrate the most commonly occurring events are under −100 pA. The amplitude data were fit with three Gaussian functions (separate fits are shown in white and the average of all three is shown in red). FIGS. 13B and 13D display the full range of amplitudes across all cells (up to −2000 pA).

Most of the evoked GABAergic events were less than −100 pA in amplitude (FIG. 13A), in remarkable agreement with known measurements of mini-IPSCs. The distribution of the peak response amplitudes was best fit with the sum of 3 Gaussian functions corresponding to −50 pA (58.14%), −92 pA (12.79%) and −147 pA (29.07%)(n=20) (FIG. 13A). Occasionally, much larger inhibitory synaptic responses were observed that exceeded more than −1 nA in amplitude but were too few to contribute to the overall Gaussian fit (FIG. 13B). In basal conditions, most of these events correspond to activity from inhibitory synapses containing α1 GABARs with a limited contribution of α3-containing GABARs. In agreement with this, the vast majority of evoked inhibitory events observed in baseline conditions exhibited relatively fast kinetics (T=11.36±0.73 ms, n=20 recordings) in good agreement with the kinetic properties of α1-containing GABARs which are significantly faster than GABARs containing α3 subunits.

The decay kinetics from all synaptic events were measured at baseline (FIGS. 13E-13F) and post-HFS (FIGS. 13G-13H) and were plotted against their amplitude. The bottom graph emphasizes the most commonly occurring events up to −500 pA. The inset in the upper right illustrates the full range of amplitudes and decay kinetics recorded. Illustrating an increase in the contribution of slow decaying events post-HFS.

High frequency stimulation (HFS) of parallel fibers was used to drive activity-dependent stimulation of extrasynaptic NMDARs invoking the nitric oxide/cGMP signaling pathway in stellate cells, as explained above (FIG. 6). Following HFS, the number of failed events decreased significantly in all recordings corresponding to a rate of 64+/−0.04% (n=20)(p=0.00, paired samples t-test) (FIGS. 12B, 12D, and 12E). Interestingly, the increase in synaptic connectivity was primarily mediated by smaller amplitude inhibitory events (FIGS. 13C-13D) that had slower decay kinetics (FIGS. 13E-13H), which is in agreement with the fact that strengthening of inhibitory synapses of cerebellar stellate cells is due to the insertion of slow-decaying, postsynaptic α3-containing GABARs. These findings also were consistent with a postsynaptic origin in inhibitory plasticity since it is difficult to explain how only small amplitude events with slow kinetics can be observed by an increase in the presynaptic release of GABA. Taken together, this data establishes that activity-dependent stimulation of NMDARs promotes an increase in connectivity of inhibitory synapses of stellate cells by the silencing of GABAergic synapses that putatively contain α3-GABARs.

### Time Latency Distributions Uncovers the Loss of Large Amplitude Synaptic Events

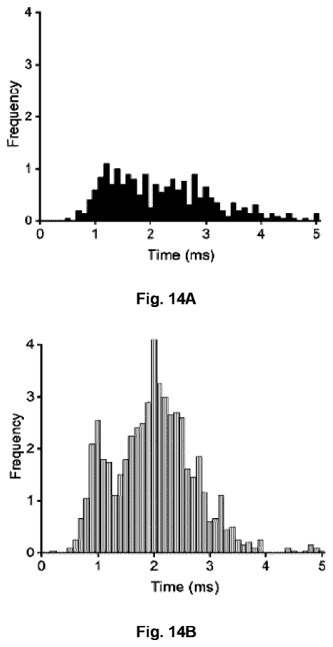

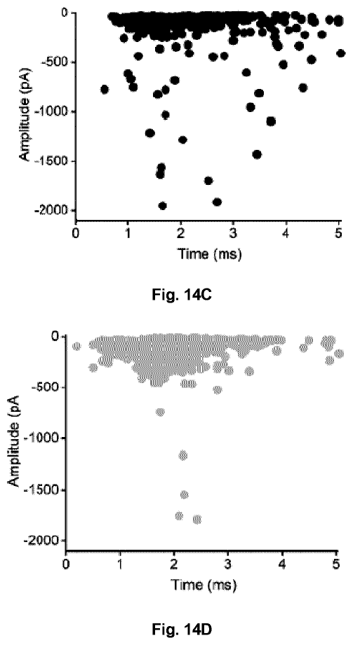

To better understand NMDAR-induced changes to GABAergic synapse plasticity, the latency and amplitude of evoked events was examined (FIGS. 14A-14D). The time latency distributions for inhibitory stellate cells tend to peak around 1-1.5 ms, but synaptic events could trail up to 2-3 ms after the presynaptic spike. Taking this into consideration, here we only events that occurred between 0.5-5.0 ms were included and analyzed after the stimulus was presented, since in the time window of the experiment appeared to represent truly evoked events.

Most of evoked events occurred within the first few milliseconds of stimulation in both baseline and post-HFS conditions (FIGS. 14A and 14B). Although, most events observed at baseline were small in amplitude, larger amplitude events were readily observed (FIG. 14C) which most likely corresponds to α1-containing GABARs. Following high frequency stimulation, the strengthening of inhibitory synapses is clearly mediated by events that are small amplitude, however a loss of the larger amplitude events was also observed. The increase in the occurrence of small amplitude events is consistent with the fact that inhibitory synapse long-term potentiation (or iLTP) is mediated by the selective insertion of α3-containing GABARs. The loss of the large amplitude events, presumably due to α1-containing GABARs, is unprecedented as it suggests that both iLTP and inhibitory synapse long-term depression (or iLTD) are simultaneously triggered in the same neuronal cell by the same stimulus. Taken together, the present findings on WT mice suggest that activity-dependent stimulation of parallel fibers triggers two distinct biochemical events in cerebellar stellate cells. L-Glu released from parallel fibers activates NMDARs and stimulates the NO/cGMP pathway triggering the selective insertion of α3-containing receptors via GABARAP into silent synaptic sites accounting for the reduction in synaptic failures. In contrast, there is also a concurrent presumptive iLTD of α1-containing GABARs accounting for the loss of large amplitude synaptic events observed following the HFS protocol (compare FIG. 14C with 14D). FIG. 15 is a schematic summarizing the presently identified working model of the separate occurrence of iLTP and iLTD at α3- and α1-GABAR synapses respectively in stellate cells of WT mice. Distinct synaptic sites for α1- and α3-containing GABAR are proposed based on the observation of silent synapses.

The present findings contrast with the conventional view of inhibitory synaptic plasticity which posits that GABA-A receptor synapses are strengthened by the accumulation of more receptors into pre-existing synaptic sites.

### α3KO Mice Lack Inhibitory LTP but Exhibit a Pronounced Inhibitory LTD

To test the veracity of the working model of iLTP and iLTD in cerebellar stellate cells, the same experiments were repeated but in mice lacking the α3-GABA receptor subunit (α3 KO mice). iLTP is lost in α3 KO mice. Accordingly, it was reasoned that if activity-dependent stimulation of glutamatergic synapses of stellate cells also induces iLTD, the depression of inhibitory synapses should be observed more clearly.

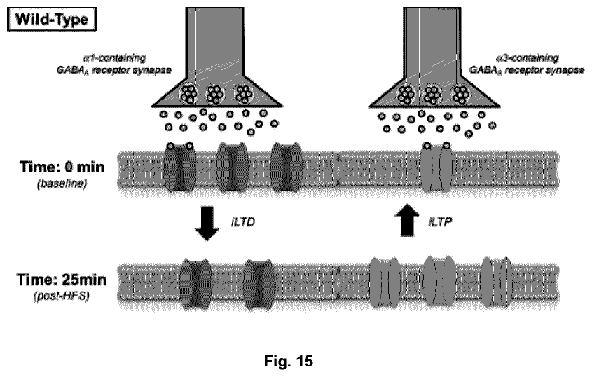

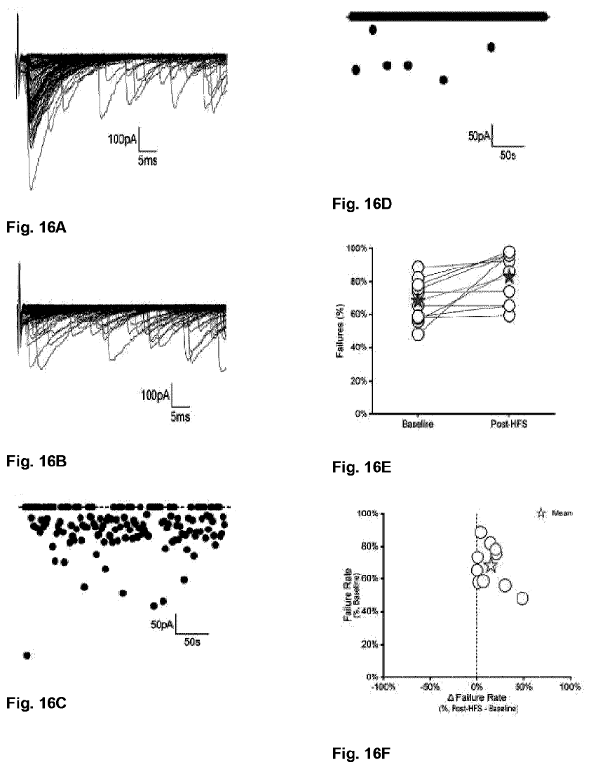

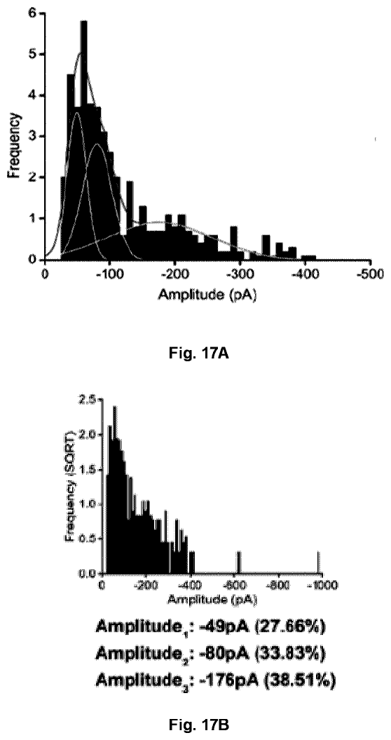

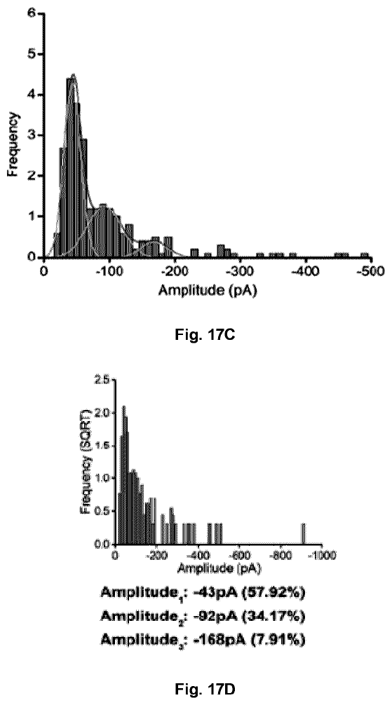

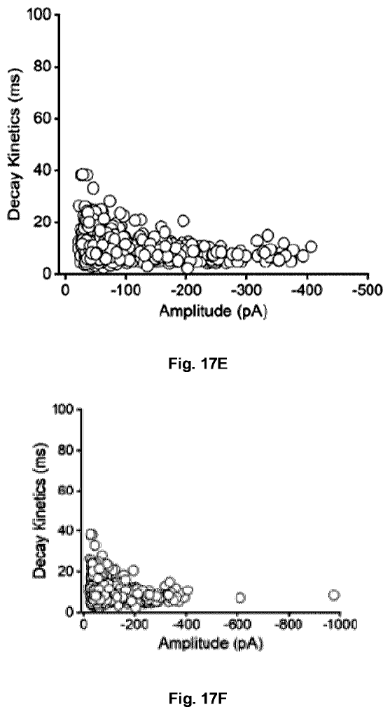

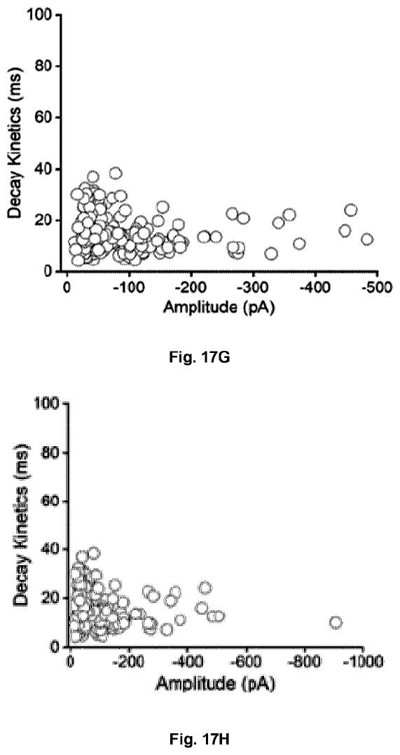

In keeping with this, the frequency (FIGS. 16A and 16C) and amplitude (FIGS. 17A-17H) of inhibitory GABAergic events recorded in α3 KO mice were similar to data from WT mice (cf FIGS. 12A-12F & 13A-13G), in agreement with the assertion that baseline synaptic transmission is primarily mediated by α1-containing GABARs. At baseline, the average failure rate was 68.33+/−4.14% (n=10), however, following the HFS protocol, rather than decreasing due to greater synapse connectivity, the failure rate increased to 82.93+/−4.85% (n=10, p=0.017, paired samples t-test) (FIGS. 16B, 16D, 16D, 16E, and 16F). As a consequence, activity dependent stimulation of glutamatergic synapses of stellate cells did not promote iLTP but rather triggered a pronounced ILTD (FIG. 16D). A comparison of the amplitude distribution of synaptic events prior to and following HFS revealed that there was a reduction in the total number of events of both small (i.e. <−100 pA) and large (>1 nA) amplitude events with little change in their decay kinetics, in contrast to the findings in WT mice.

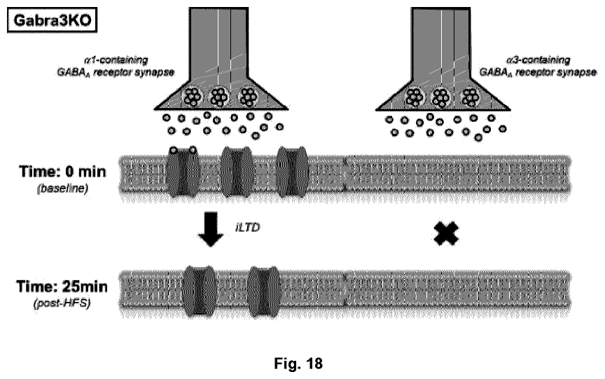

Taken together, these results support the working model of GABAergic synaptic plasticity in stellate cells whereby NMDARs are unable to promote the insertion of α3-containing GABARs into silent inhibitory synaptic sites (FIG. 18). The absence of iLTP, uncovers iLTD at α1-GABAR synapses which would normally be masked in WT mice due to dominance of iLTP.

FIG. 18 is a schematic summarizing the nature of inhibitory synaptic plasticity in mice lacking the α3-GABAR subunit. Genetic deletion of α3 subunit eliminates the ability of NMDARs expressed by stellate cells to induce iLTP via NO/cGMP signaling. The loss of iLTP uncovers the marked expression of iLTD at α1-containing GABAR synapses.

Fmr1 KO Mice Also Lack iLTP but Exhibit a More Enhanced Expression of ILTD

Observations made (data not shown) have demonstrated that in a preclinical model of the neurodevelopmental disorder, Fragile X syndrome (FXS), extrasynaptic NMDARs have a weak functional expression. FXS mice lack expression of the RNA binding protein, Fragile X messenger ribonucleoprotein (FMRP) due to the genetic deletion of the Fmr1 gene. In WT mice it was shown that iLTP in stellate cells relies on the robust activation of NMDARs to trigger NO/cGMP signaling promoting the insertion of α3-GABARs. Given this, it was hypothesized that Fmr1 KO mice would also lack iLTP, like α3-KO mice, but in this case, the absence of iLTP would be due to the hypofunction in extrasynaptic NMDARs. Whether the expression of iLTD is similarly dependent on NMDARs or mediated by another glutamatergic mechanism is still not clear.

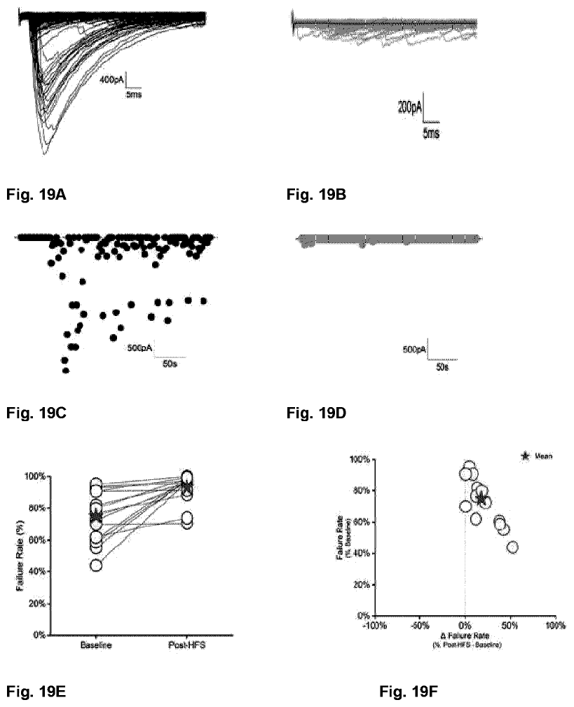

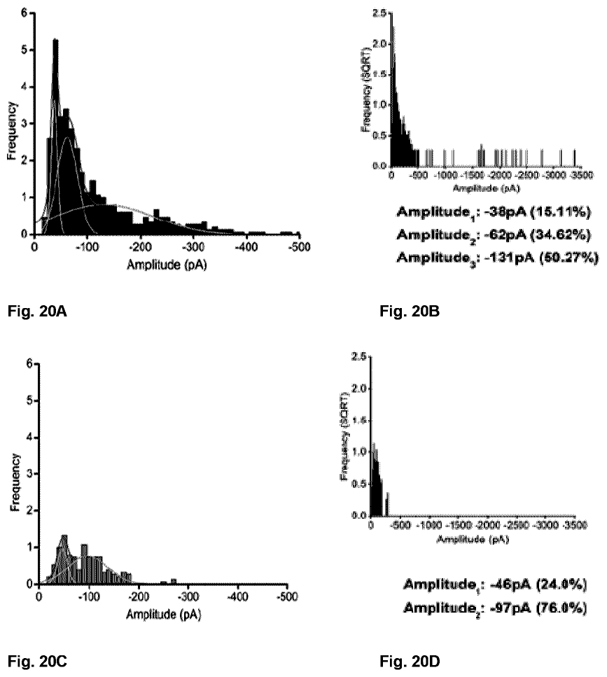

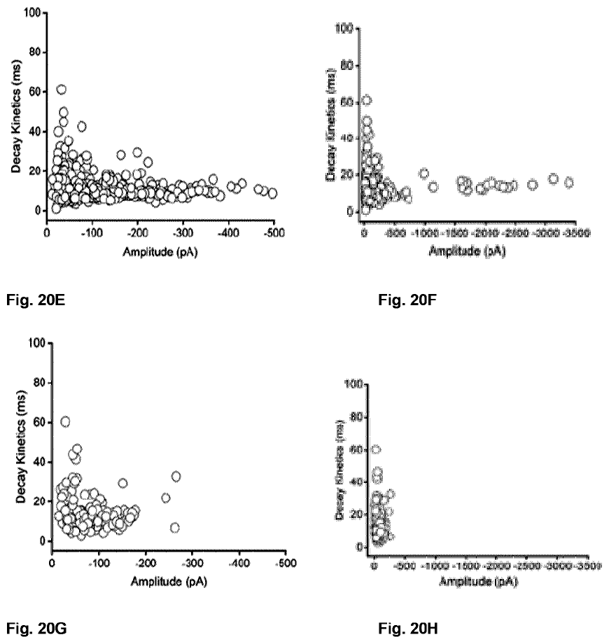

To examine these mechanisms, minimal stimulation experiments were performed on stellate cells from Fmr1 KO mice and failure rates were measured before and after HFS of parallel fibers (FIGS. 19A-19F). In baseline conditions, evoked GABAergic events in Fmr1 KO mice were similar in amplitude to inhibitory events recorded in WT mice (FIGS. 20A-20B) with a failure rate of 74.98+/−4.17% (n=15) (FIGS. 20A, 20B, 20E and 20F). HFS of parallel fibers failed to induce iLTP, as anticipated due to the hypofunction in NMDARs, but rather promoted an exaggerated form of iLTD (FIGS. 19B & 19D). The average failure rate significantly increased to 93.16+/−2.32% (n=15, p=0.001, paired samples t-test) after the HFS protocol representing a substantial decrease in synaptic connectivity and strength. Unexpectedly, HFS of glutamatergic synapses not only affected evoked GABAergic events but also diminished spontaneous GABAR events (FIG. 19B) which was not observed in α3 KO mice (FIG. 16B). Consequently, we concluded that the mechanism that induces iLTD in stellate cells from Fmr1 KO mice is much more exaggerated in nature as it has a global effect on GABAergic transmission. Finally, post-HFS the decay kinetics did not change (M=12.38+/−1.44 ms, n=15, p=0.514, paired samples t-test) (FIGS. 20C-20D and 20G-20H). Illustrating that, similar to the α3KO mice, there is no change in the occurrence of small amplitude or slow decaying synaptic events, but instead an overall reduction in all synaptic activity. Furthermore, the large amplitude and fast decaying events are almost completely eradicated following HFS, indicating an enhanced iLTD in these mice.

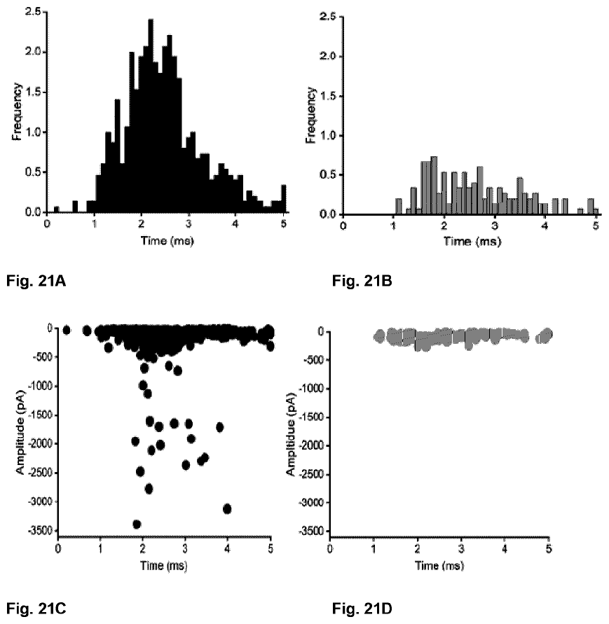

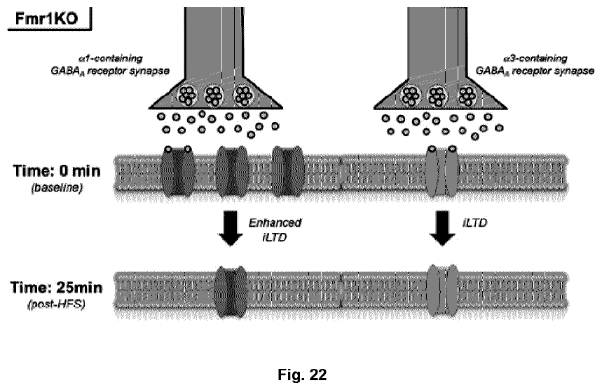

In keeping with this, plots of the latency time of the synaptic events revealed that both large and small amplitude synaptic were almost eliminated following HFS (FIGS. 21A-21D). Taken together, the data on Fmr1 KO mice reveals that the basal property of GABAergic transmission is similar in both WT and FXS mice. However, following activity-dependent stimulation of parallel fibers to promote the release of the neurotransmitter, L-Glu, rather than promoting iLTP and iLTD of GABARs synapses, an exaggerated form of iLTD only is observed. These findings reveal for the first time that the mechanisms that would normally lead to the strengthening of GABAergic synapses, not only fails to promote iLTP, but instead, triggers an exaggerated form of iLTD. The schematic shown in FIG. 22 which summarizes the salient points of these observations.

Overall, these results indicate two important findings in the Fmr1 KO mice. First, they display a lack of α3-mediated iLTP following HFS due to the hypofunction in NMDAs. Secondly, Fmr1 KO mice possess enhanced iLTD of α1 receptor synapses reflecting the substantial reduction in the large amplitude, fast decaying, and fast onset GABAergic events. The Fmr1 KO phenotype displays an even more robust synaptic depression than the 3 KO mice, since it is not only just the single synapse that is being weakened (the evoked events), but the entire cell (including spontaneous activity). These findings infer a consequential global double knock-on effect, whereby Fmr1 KO mice are not only losing the ability to strengthen GABAergic signaling, but that they are also losing the total number of GABARs from the cell surface. As a result, Fmr1 KO mice experience diminished inhibition and a lack of synapse connectivity and strength.

As a result, mechanisms that normally trigger the strengthening of inhibitory synapses instead promote a worsening of overall inhibitory signaling response due to two distinct processes as outlined in FIG. 22. Overall, these results show the decreased expression of GABAR subtypes (particularly α1 and α3) in the cortex of Fmr1 KO mice, which leads to a reduction in inhibitory strength. Therefore, the present findings explain why canonical forms of treatment utilizing benzodiazepines and other GABAergic drugs have limited efficacy in FXS, as there are not enough GABARs for the drug to fully exert its effect. The mechanism(s) that mediate α1-iLTD were next examined to identify the downstream signaling pathways that lead to this impairment.

Block of mGluR5 Signaling Attenuates Fmr1KO Enhanced iLTD

Patients with FXS and mice lacking FMRP, both display overactive signaling of metabotropic glutamate receptors (mGluRs), most importantly mGluR5. mGluR5 and mGluR1 both belong to the group 1 of mGluRs (Gp1) and have been implicated in mediating iLTD of excitatory synapses of the cerebellum and hippocampus. The hippocampal and cerebellar mGluR-LTD are altered in Fmr1 KO mice, since FMRP regulates mGluR-dependent protein synthesis and plasticity. Specifically, enhanced mGluR-LTD at the PF-PC synapse results from a loss of FMRP in postsynaptic Purkinje neurons and is associated with deficits in cerebellar-mediated learning, in both Fmr1 KO mice and FXS patients. However, most of the focus of previous research was on how mGluR-LTD affects excitatory transmission and AMPAR surface expression. Whether mGluR signaling also promotes LTD of inhibitory synapses had yet to be investigated.

At glutamatergic synapses, mGluR5 antagonists or genetic reduction of mGluR5 reverse multiple phenotypes in Fmr1 KO mice. Gp1 mGluRs are commonly linked to activation of phospholipase C (PLC), generation of inositol trisphosphate (IP3), release of Ca²⁺ from intracellular stores, and activation of PKC, which are all required for cerebellar mGluR-LTD. Therefore, the hypothesis that overactive signaling by Gp1 mGluRs could be responsible for the enhanced iLTD observed at inhibitory synapses of Fmr1 KO mice was tested. To do this, the mGluR5 negative allosteric modulator, MPEP, was included in the external aCSF solution to inhibit mGluR5 signaling and block the PLC/Gq/IP3/diacylglycerol (DAG) second messenger pathway.

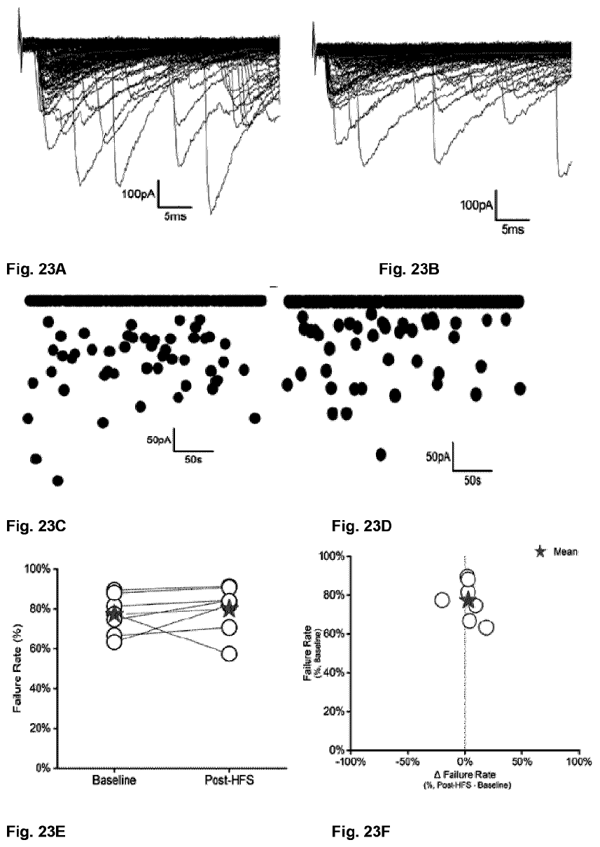

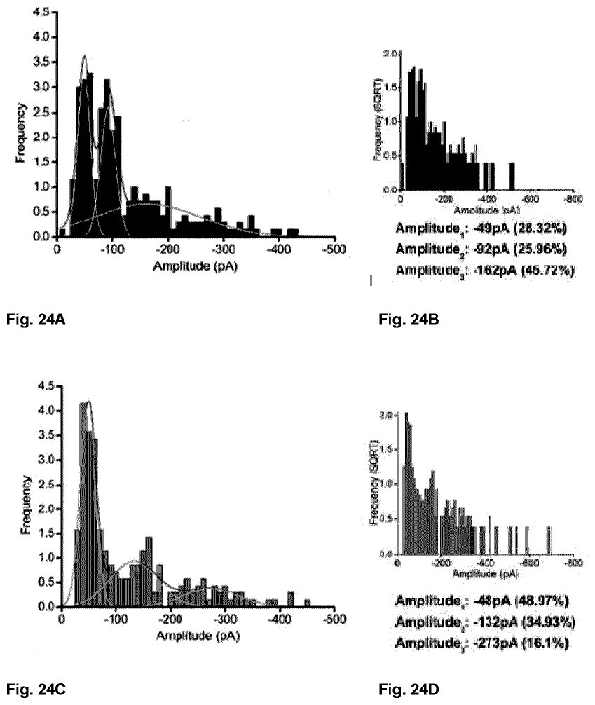

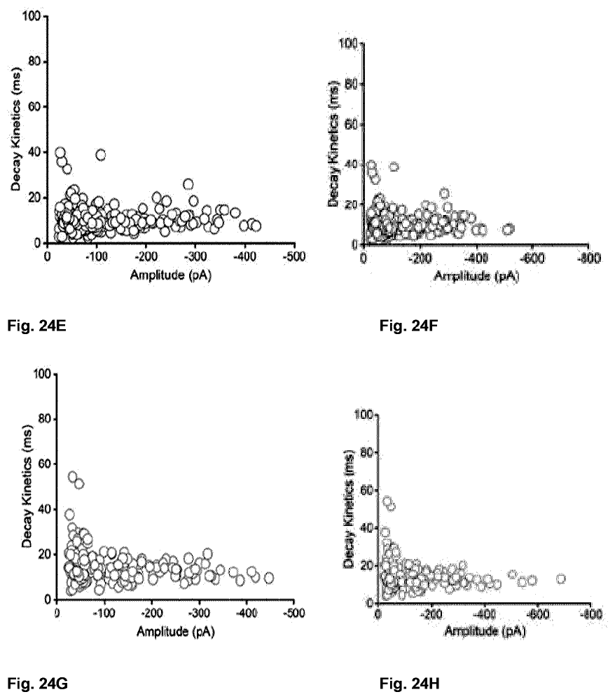

In the presence of 10 μM MPEP to block mGluR5 activity, both the failure rate of GABAergic transmission (Mean, 77.21+/−3.75%, n=7) (FIGS. 23A-23F) and amplitude of evoked synaptic events (FIGS. 24A-24H) were similar to baseline levels in the absence of MPEP suggesting that mGluR5 antagonism does not affect basal synaptic transmission. Following HFS of parallel fibers, the failure rate of GABA-evoked events (Mean, 79.98+/−4.58%, n=7, p=0.553, paired samples t-test), and distribution of event amplitudes were unchanged (FIGS. 24A-24F). The failure rate measurement was very consistent across all cells, such that none of the cells significantly changed their failure rate from baseline to post-HFS (FIGS. 24A-24F). These results demonstrate that blockage of mGluR5 signaling by MPEP prevents the onset of iLTD in cerebellar stellate cells from Fmr1 KO mice.

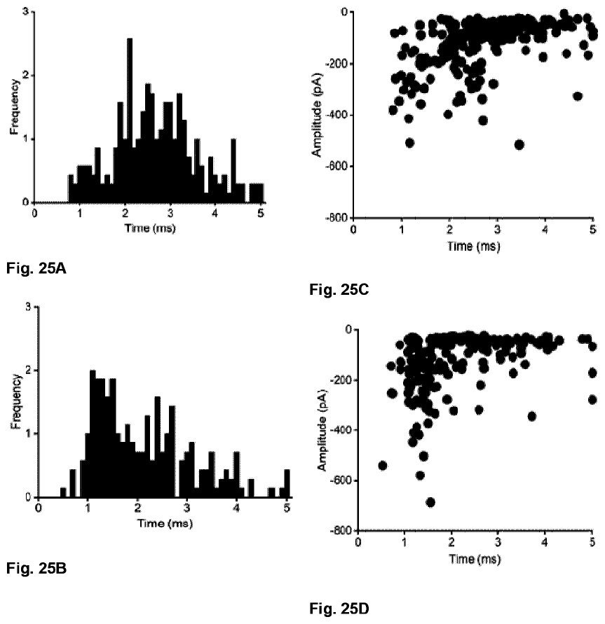

Lastly, the time latencies for all the synaptic events were measured and elucidated that during baseline the events appeared randomly (Mean=2.78+/−0.24 ms, n=7) (FIG. 24A), but post-HFS this got slightly faster (Mean=2.32+/−0.29 ms, n=7, p=0.136, paired samples t-test) (FIGS. 25A-25D). This revealed a rescue of the large amplitude and fast onset events. Based on these results, the profile of events that were rescued post-HFS matches the α1-mediated events. Therefore, here MPEP was beneficial in preventing the onset of iLTD.

Inhibition of PDE5 Restores α3-Mediated iLTP to Fmr1 KO Mice

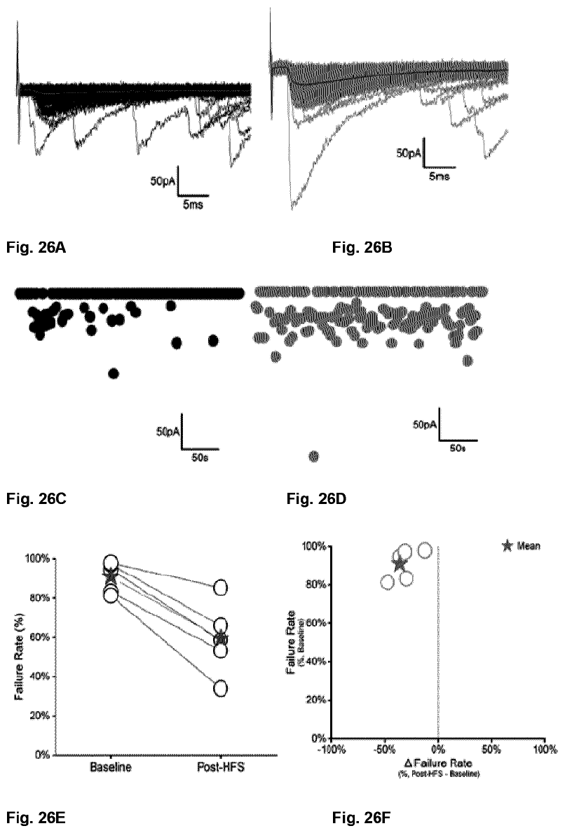

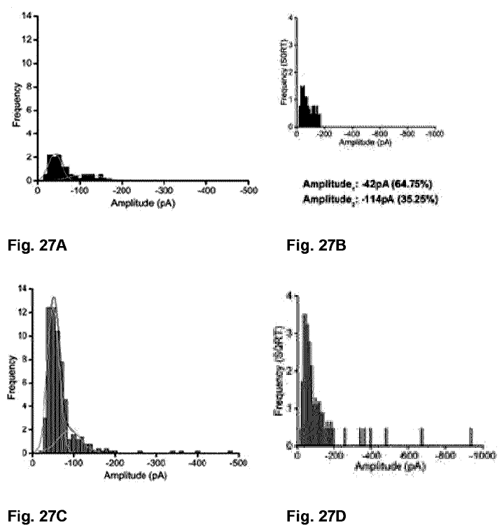

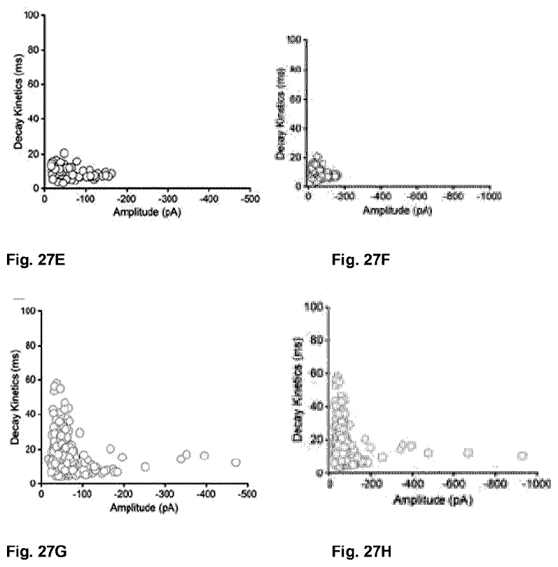

Fmr1 KO mice display deficits in attaining inhibitory LTP (iLTP) because the pathways that are involved in synapse strengthening are altered or depressed. Furthermore, NMDAR currents in Fmr1 KO mice have also been shown to be reduced, reflecting that these mice have fewer synaptic receptors to help mediate iLTP. However, there is currently a lack of evidence regarding how iLTP is mediated in Fmr1 KO mice. The NMDA response in Fmr1 KO mice is also reduced and this leads to a defect in GABAergie plasticity (data not shown). Through understanding how GABAR synapses strengthen in WT mice, utilizing NO and cGMP, a therapeutic drug was identified by targeting phosphodiesterase 5 (PDE5) for inhibition using sildenafil which prolongs the half-life of cytoplasmic cGMP. In baseline conditions, all cells exhibited a high rate of failure (Mean=90.9+/−3.57%, n=5) when 10−100 μM sildenafil was included in the patch electrode establishing that PDE5 inhibition does not affect the basal properties of inhibitory transmission (FIGS. 26A-26F), much like MPEP. The amplitude of evoked events were also similar to WT control conditions (FIGS. 27A-B & 27E-27F). Following HFS of parallel fibers, a remarkable restoration of iLTP was observed in stellate cells from Fmr1 KO whereby an increased in synapse connectivity was observed indicated by the occurrence of fewer failed events (Mean, 59.44+/−8.36%, n=5, p=0.005, paired samples t-test). In keeping with this, many more smaller amplitude evoked events were observed with slow decay kinetics (FIGS. 27C-D & 27G-27H), a characteristic feature of iLTP and indicative of the emergence of synapses containing α3-containing GABARs. Taken together, these results suggest that sildenafil can sufficiently augment α3 mediated iLTP.

Inhibitory Combination of Targeting PDE5 and mGluRs Enhances iLTP by Blocking iLTD

The present disclosure has established that inhibition of mGluR5 signaling prevents the exaggerated form of iLTD found in stellate cells (FIGS. 23A-23F, 24A-24H, 25A-25D) whereas experiments with the PDE5 inhibitor, sildenafil, reveals that prolongation of cGMP in stellate cells promotes α3-mediated iLTP (FIGS. 26A-26F and 27A-27H). Given that the exaggerated form of iLTD is presumably still present in experiments with sildenafil, the blocking both mGluR5 receptors and PDE5 was tested to determine whether it would elicit a greater iLTP response.

A bath incubation with MPEP and the inclusion of sildenafil in the patch electrode did not affect basal synaptic transmission with failure rates of 89.23+/−4.52% (n=9) (FIGS. 28A, 28C, and 28E) with synaptic events whose amplitude were similar to WT control (FIGS. 29A-29B & 29E-29F). Following HFS, however, the failure rates decreased significantly to 63.39+/−7.40% (n=9, p=0.002, paired samples t-test) (FIGS. 28B, 28D, and 28F) with a marked increase in the total number of events that include events of large amplitude (FIGS. 29C-29D & 29G-29H). Next, the averaged amplitudes from all cells started off small during baseline (M=−97.19+/−12.89 pA, n=9) (FIGS. 29A-29B). Furthermore, all cells displayed fast decay kinetics (M=9.47+/−0.91 ms, n=9) (FIGS. 29E-29F) at baseline that slowed significantly post-HFS (M=15.30+/−1.43 ms, n=9, p=0.003, paired samples t-test) (FIGS. 29G-29H). Lastly, the average time latency during baseline for all cells was (M=2.32+/−0.22 ms, n=9) (FIG. 30A), and this did not change too much post-HFS (M=1.94+/−0.15 ms, n=9, p=0.059, paired samples t-test) (FIGS. 30A-30D).

Overall, these results utilizing a combination of both MPEP and sildenafil show that they are very effective in correcting synaptic connectivity defects in cerebellar stellate cells. Sildenafil can promote the uncovering of silent α3-containing GABAR synapses that gives rise to iLTP. This plasticity mechanism is characterized by an increase in the number of small amplitude (under −100 pA) events that have slow decay kinetics. MPEP was able to prevent the exaggerated form of iLTD of the α1 synapses. The rescue in α1-mediated iLTD is characterized by the occurrence of the large amplitude events post-HFS. Therefore, taken together, this combination of drugs was sufficient to correct deficits in GABAergic transmission by targeting two bifurcating pathways. The first is implicated in α3 mediated iLTP and increases GABAergic synaptic strength. The second prevents α1-iLTD through blocking excessive activity of Gp1 mGluRs with MPEP. The drug combination is therefore a therapeutic strategy in the treatment of Fragile X syndrome.

Additional experiments (data not shown) were conducted using the HFS protocol on WT mice described above in the presence of APV to block NMDARs. Results showed that on average the failure rates of all cells post-HFS, did not change too much, but only slightly decreased by ˜5%. This finding indicates that antagonizing NMDARs was able to attenuate the profound iLTP normally observed in WT.

iLTP requires an elevation of cytosolic Ca²⁺. In keeping with this, the HFS protocol was performed on WT mice in the presence of a high BAPTA internal solution to chelate cytosolic Ca²⁺. The results confirmed a similar but more significant effect than APV, such that on average all cells, did not change their failure rate post-HFS (data not shown). This observation indicates that calcium is necessary for the induction of iLTP. To examine the role of PKC, the kinase inhibitor, Gö6983 was tested on WT mice. Again, the results indicated that in all cells, the failure rate from baseline to post-HFS did not change. The results suggest that inhibition of PKC eliminates the induction of iLTP by the HFS protocol. Thus, some essential proteins implicated in the iLTP pathway, demonstrate promising results resulting that the same sequential pathway is being stimulated by NMDARs.

To confirm that MPEP is mediating its effect by preventing iLTD of α1-containing GABARs, additional HFS protocol experiments were performed on α3 KO mice. The results show that on average all cells actually slightly decreased their failure rate (or increased their synaptic connectivity) (data not shown). Considering that α3 GABARs are absent in these mice, it suggests that the normal α1 iLTD that typically occurs during synaptic strengthening was rescued.

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Alexander R P D, Bowie D. Intrinsic plasticity of cerebellar stellate cells is mediated by NMDA receptor regulation of voltage-gated Nar channels. J Physiol. 2021 Jan; 599(2):647-665. doi; 10.1113/JP280627. Epub 2020 Nov. 16. PMID: 33146903
Domek-Łopacińska K, Strosznajder J B. Cyclic GMP metabolism and its role in brain physiology. J Physiol Pharmacol. 2005 Mar; 56 Suppl 2:15-34. PMID: 16077188.
Larson E A, Accardi M V, Wang Y, D'Antoni M, Karimi B, Siddiqui T J, Bowie D. Nitric Oxide Signaling Strengthens Inhibitory Synapses of Cerebellar Molecular Layer Interneurons through a GABARAP-Dependent Mechanism. J Neurosci. 2020 Apr. 22; 40(17):3348-3359. doi: 10.1523/JNEUROSCI.2211-19.2020. Epub 2020 Mar. 13. PMID: 32169968
Zeisel, A et al. (2018) Molecular Architecture of the Mouse Nervous System. Cell 174, 999-1014 e1022 and, also, mousebrain.org.

What is claimed is:

1. A method of mitigating at least one symptom of Fragile X syndrome in an individual in need thereof, the method comprising administering a therapeutically effective amount of one or more inhibitor of one or more phosphodiesterase to the individual to mitigate the at least one symptom, wherein the one or more phosphodiesterase is capable of hydrolyzing cGMP.

2. The method of claim 1, wherein the one or more phosphodiesterase comprises a cGMP-selective phosphodiesterase.

3. The method of claim 2, wherein the one or more phosphodiesterase comprises phosphodiesterase 5.

4. The method of claim 3, wherein the one or more inhibitor comprises sildenafil or a pharmaceutically acceptable salt thereof.

5. The method of claim 1 or 2, wherein the one or more phosphodiesterase is further capable of hydrolyzing cAMP.

6. The method of claim 5, wherein the one or more phosphodiesterase comprises phosphodiesterase 1, 2 and/or 10.

7. The method of any one of claims 1 to 6, further comprising administering a therapeutically effective amount of a mGluR5 blocking agent.

8. The method of claim 7, wherein the mGluR5 blocking agent is an antagonist of mGluR5 or a negative allosteric modulator of mGluR5.

9. The method of claim 7 or 8, wherein the blocking agent is selected from the group consisting of 2-Methyl-6-(phenylethynyl)-pyridine (MPEP), methyl (3aR,4S,7aR)-4-hydroxy-4-[2-(3-methylphenyl)ethynyl]octahydro-1H-indole-1-carboxylate (mavoglurant), N-(3-Chlorophenyl)-N′-[1-methyl-4-oxo-4,5-dihydro-1H-imidazol-2-yl)urea (fenobam), 3-((2-Methyl-1,3-thiazol-4-yl)ethynyl)pyridine (MTEP), 6-methyl-2-(phenylazo)-3-pyridinol (SIB-1757), (E)-2-methyl-6-(2-phenylethenyl)pyridine (SIB-1893), basimglurant (2-chloro-4-{2-[1-(4-fluorophenyl)-2,5-dimethyl-1H-imidazol-4-yl]ethynyl}pyridine), 6-Fluoro-2-(4-(pyridin-2-yl)but-3-yn-1-yl)imidazo(1,2-a)pyridine (dipraglurant), 3-fluoro-5-[3-(5-fluoropyridin-2-yl)-1,2,4-oxadiazol-5-yl]benzonitrile (AZD 9272), 2-[(3-Fluorophenyl)ethynyl]-4,6-dimethyl-3-pyridinamine (raseglurant), N-(5-Fluoropyridin-2-yl)-6-methyl-4-(pyrimidin-5-yloxy)picolinamide (VU0424238), GRN-529 ([4-(Difluoromethoxy)-3-[2-(2-pyridinyl)ethynyl]phenyl](5,7-dihydro-6H-pyrrolo[3,4-b]pyridin-6-yl)-methanone), (6-Bromopyrazolo[1,5-a]pyrimidin-2-yl)[(1R)-1-methyl-3,4-dihydro-2(1H)-isoquinolinyl]methanone (remeglurant), (2S)-2-Amino-2-[(1S,2S)-2-carboxycycloprop-1-yl]-3-(xanth-9-yl)propanoic acid (LY-341495), GET73 (4-methoxy-N-[[4-(trifluoromethyl)phenyl]methyl]butanamide), arbaclofen ((3R)-4-amino-3-(4-chlorophenyl)butanoic acid), HTL-0014242 ((3-Chloro-5-[6-(5-fluoropyridin-2-yl)pyrimidin-4-yl]benzonitrile)), 2-chloro-N-[2-methoxy-4-(pyridin-2-yldiazenyl)phenyl]benzamide (Alloswitch1), PAM12,4-chloro-N-(6-(pyrimidin-5-yloxy)pyrazin-2-yl)picolinamide (VU-0431316), N-(4,4-dimethylcyclohexyl)pyrido[1′,2′:1,5]pyrazolo[4,3-d]pyrimidin-4-amine (VU-0467558), VU-0463841 (1-(5-chloropyridin-2-yl)-3-(3-cyano-5-fluorophenyl)urea), AP-612, LCGM-10, (3-fluorophenyl)[2-(5-fluoropyridin-2-yl)]-6,7-dihydoro[1,3]oxazolo[4,5-c]pyridin-5(4H)-yl]methanone (DSR-98776), EPX-105287, (aS)-α-Amino-α-[(1R,2R)−2-carboxycyclopropyl]-9H-xanthene-9-propanoic acid (LY-344545), MRZ-8676 (6,6-dimethyl-2-(2-phenylethynyl)-7,8-dihydroquinolin-5-one), 3-((4-(4-chlorophenyl)-7-fluoroquinolin-3-yl)sulfonyl)benzonitrile (RGH-618), 5-(3-chlorophenyl)-3-[(1R)-1-[(4-methyl-5-pyridin-4-yl-1,2,4-triazol-3-yl)oxy]ethyl]-1,2-oxazole (AZD-2066), AZD-2516, AZD-6538 (6-[5-(3-cyano-5-fluorophenyl)-1,2,4-oxadiazol-3-yl]pyridine-3-carbonitrile), and (RS)-α-methyl-4-carboxyphenylglycine ((RS)-MCPG).

10. The method of any one of claims 1 to 9, wherein the one or more inhibitor after having been administered to the individual is capable of facilitating in at least one brain region, when compared to the corresponding at least one brain region in a control individual:
a) intrinsic plasticity via a sodium channel;
b) vasodilation; and/or
c) GABAergic inhibitory synaptic plasticity.

11. The method of claim 10, wherein the brain region is the cerebellum.

12. The method of any one of claims 1 to 11, wherein the individual is a human.

13. The method of any one of claims 1 to 12, wherein the individual is a child.

14. The method of any one of claims 1 to 13, wherein the individual is a baby.

15. The method of any one of claims 1 to 14, wherein the individual has been diagnosed with Fragile X Syndrome.

16. The method of any one of claims 1 to 15, wherein the at least one symptom comprises:
a) hyperactivity;
b) male aggression;
c) anxiety;
d) a learning deficit;
e) a memory deficit;
f) a sensory deficit;
g) a sleep abnormality and/or
h) a repetitive behavior.

17. The method of any one of claims 1 to 16 comprising administering an effective amount of at least two phosphodiesterase Inhibitors to the individual.

18. A method for determining the usefulness of a test agent in the mitigation of a symptom of Fragile X Syndrome (FXS), the method comprising contacting the test agent with a test cell capable of expressing neuronal nitric oxide synthase (nNOS), measuring a test level of activity of nNOS in the presence of the test agent and determining that the test agent is useful if the test level of activity of nNOS is higher than a control level of activity obtained from a control cell.

19. The method of claim 18, wherein the test agent is capable of inhibiting the activity of at least one phosphodiesterase capable of hydrolyzing cGMP.

20. The method of claim 19, wherein the at least one phosphodiesterase comprises a selective cGMP phosphodiesterase.

21. The method of claim 20, wherein the at least one phosphodiesterase comprises phosphodiesterase 5.

22. The method of claim 19, wherein the at least one phosphodiesterase is further capable of hydrolyzing CAMP.

23. The method of claim 22, wherein the at least one phosphodiesterase comprises phosphodiesterase 1, 2 or 10.

24. The method of any one of claims 18 to 23, wherein test cell is capable of expressing a N-methyl-D-aspartate receptor (NMDAR) and the method comprises measuring a test level of activity of NMDAR in the presence of the test agent and determining that the test agent is useful if the test level of activity of the NMDAR is lower than a control level of activity obtained from a control cell.

25. The method of any one of claims 18 to 23 further comprising:
a) contacting the test agent with a test brain sample comprising the test cell in order to obtain a treated brain sample;
b) measuring, in the treated brain sample, one or more of the following to obtain one or more test values:
i) intrinsic plasticity via a sodium channel;
ii) a degree of vasodilation; and/or
iii) a level of GABAergic inhibitory synaptic plasticity;
c) comparing the at least one test value obtained in (b) with a corresponding at least one control value obtained with a control brain sample comprising the control cell; and
d) determining that the test agent is useful if the one or more test value is increased with respect to the one or more control value;
wherein:
the test brain sample and the control brain sample are derived from an individual having fragile X syndrome (FXS) or an animal model of FXS.

26. The method of claim 25, wherein the animal model is a mouse model.

27. The method of claim 26, wherein the mouse model comprises a homozygous deletion of the Fmr1 gene.

28. The method of any one of claims 25 to 27, wherein the test brain sample and the control brain sample are derived from the same individual having FXS or the same animal model of FXS.

29. The method of any one of claims 25 to 28 comprising measuring intrinsic plasticity via a sodium channel by determining the action current of cell-attached recordings.

30. The method of any one of claims 25 to 29 comprising measuring the degree of vasodilation by determining the size and/or the volume of cerebral blood vessels.

31. The method of any one of claims 25 to 30 comprising measuring GABAergic inhibitory synaptic plasticity with current-clamp recordings.

32. The method of any one of claims 25 to 30 comprising measuring GABAergic inhibitory synaptic plasticity with voltage-clamp recordings.

33. A method of mitigating at least one symptom of Fragile X syndrome, GRIN disorder, SynGAP1 intellectual disability or Phelan-McDermid syndrome in an individual in need thereof, the method comprising administering a therapeutically effective amount of one or more inhibitor of one or more phosphodiesterase to the individual to mitigate the at least one symptom, wherein the one or more phosphodiesterase is capable of hydrolyzing cGMP.

34. The method of claim 33, wherein the one or more phosphodiesterase comprises a cGMP-selective phosphodiesterase.

35. The method of claim 34, wherein the one or more phosphodiesterase comprises phosphodiesterase 5.

36. The method of claim 35, wherein the one or more inhibitor comprises sildenafil or a pharmaceutically acceptable salt thereof.

37. The method of claim 33 or 34, wherein the one or more phosphodiesterase is further capable of hydrolyzing cAMP.

38. The method of claim 37, wherein the one or more phosphodiesterase comprises phosphodiesterase 1, 2 and/or 10.

39. The method of any one of claims 1 to 38, further comprising administering a therapeutically effective amount of a mGluR5 blocking agent.

40. The method of claim 39, wherein the mGluR5 blocking agent is an antagonist of mGluR5 or a negative allosteric modulator of mGluR5.

41. The method of claim 38 or 39, wherein the blocking agent is selected from the group consisting of 2-Methyl-6-(phenylethynyl)-pyridine (MPEP), methyl (3aR,4S,7aR)-4-hydroxy-4-[2-(3-methylphenyl)ethynyl]octahydro-1H-indole-1-carboxylate (mavoglurant), N-(3-Chlorophenyl)-N′-(1-methyl-4-oxo-4,5-dihydro-1H-imidazol-2-yl)urea (fenobam), 3-((2-Methyl-1,3-thiazol-4-yl)ethynyl)pyridine (MTEP), 6-methyl-2-(phenylazo)-3-pyridinol (SIB-1757), (E)-2-methyl-6-(2-phenylethenyl)pyridine (SIB-1893), basimglurant (2-chloro-4-{2-[1-(4-fluorophenyl)-2,5-dimethyl-1H-imidazol-4-yl]ethynyl}pyridine), 6-Fluoro-2-(4-(pyridin-2-yl)but-3-yn-1-yl)imidazo(1,2-a)pyridine (dipraglurant), 3-fluoro-5-[3-(5-fluoropyridin-2-yl)-1,2,4-oxadiazol-5-yl]benzonitrile (AZD 9272), 2-[(3-Fluorophenyl)ethynyl]-4,6-dimethyl-3-pyridinamine (raseglurant), N-(5-Fluoropyridin-2-yl)-6-methyl-4-(pyrimidin-5-yloxy)picolinamide (VU0424238), GRN-529 ([4-(Difluoromethoxy)-3-[2-(2-pyridinyl)ethynyl]phenyl](5,7-dihydro-6H-pyrrolo[3,4-b]pyridin-6-yl)-methanone), (6-Bromopyrazolo[1,5-a]pyrimidin-2-yl)[(1R)-1-methyl-3,4-dihydro-2(1H)-isoquinolinyl]methanone (remeglurant), (2S)-2-Amino-2-[(1S,2S)-2-carboxycycloprop-1-yl]-3-(xanth-9-yl)propanoic acid (LY-341495), GET73 (4-methoxy-N-[[4-(trifluoromethyl)phenyl]methyl]butanamide), arbaclofen ((3R)-4-amino-3-(4-chlorophenyl)butanoic acid), HTL-0014242 ((3-Chloro-5-[6-(5-fluoropyridin-2-yl)pyrimidin-4-yl]benzonitrile)), 2-chloro-N-[2-methoxy-4-(pyridin-2-yldiazenyl)phenyl]benzamide (Alloswitch1), PAM12,4-chloro-N-(6-(pyrimidin-5-yloxy)pyrazin-2-yl)picolinamide (VU-0431316), N-(4,4-dimethylcyclohexyl)pyrido[1′,2′:1,5]pyrazolo[4,3-d]pyrimidin-4-amine (VU-0467558), VU-0463841 (1-(5-chloropyridin-2-yl)-3-(3-cyano-5-fluorophenyl)urea), AP-612, LCGM-10, (3-fluorophenyl)[2-(5-fluoropyridin-2-yl)]-6,7-dihydoro[1,3]oxazolo[4,5-c]pyridin-5(4H)-yl]methanone (DSR-98776), 3-((4-(4-chlorophenyl)-7-fluoroquinolin-3-yl)sulfonyl)benzonitrile (RGH-618), 5-(3-chlorophenyl)-3-[(1R)-1-[(4-methyl-5-pyridin-4-yl-1,2,4-triazol-3-yl)oxy]ethyl]-1,2-oxazole (AZD-2066), AZD-2516, AZD-6538 (6-[5-(3-cyano-5-fluorophenyl)-1,2,4-oxadiazol-3-yl]pyridine-3-carbonitrile), and (RS)-α-methyl-4-carboxyphenylglycine ((RS)-MCPG).

42. A method of mitigating at least one symptom of Fragile X syndrome, GRIN disorder, SynGAP1 intellectual disability or Phelan-McDermid syndrome in an individual in need thereof, the method comprising administering a therapeutically effective amount of one or more inhibitor of one or more phosphodiesterase and a therapeutically effective amount of a mGluR5 blocking agent to the individual to mitigate the at least one symptom, wherein the one or more phosphodiesterase is capable of hydrolyzing cGMP.

43. A method of preventing onset of Fragile X syndrome, GRIN disorder, SynGAP1 intellectual disability or Phelan-McDermid syndrome in an individual in need thereof, the method comprising administering a therapeutically effective amount of one or more inhibitor of one or more phosphodiesterase and a therapeutically effective amount of a mGluR5 blocking agent to the individual to mitigate the at least one symptom, wherein the one or more phosphodiesterase is capable of hydrolyzing cGMP.

44. Use of one or more inhibitor of one or more phosphodiesterase in the treatment of Fragile X syndrome, GRIN disorder, SynGAP1 intellectual disability or Phelan-McDermid syndrome in an individual in need thereof.

45. Use of one or more inhibitor of one or more phosphodiesterase and a mGluR5 blocking agent in the treatment of Fragile X syndrome, GRIN disorder, SynGAP1 intellectual disability or Phelan-McDermid syndrome in an individual in need thereof.

46. Use of one or more inhibitor of one or more phosphodiesterase in the manufacture of a medicament for the treatment of Fragile X syndrome, GRIN disorder, SynGAP1 intellectual disability or Phelan-McDermid syndrome in an individual in need thereof.

47. Use of one or more inhibitor of one or more phosphodiesterase and a mGluR5 blocking agent in the manufacture of a medicament for the treatment of Fragile X syndrome, GRIN disorder, SynGAP1 intellectual disability or Phelan-McDermid syndrome in an individual in need thereof.

48. Use of one or more inhibitor of one or more phosphodiesterase for the alleviation of a symptom of Fragile X syndrome, GRIN disorder, SynGAP1 intellectual disability or Phelan-McDermid syndrome in an individual in need thereof.

49. Use of one or more inhibitor of one or more phosphodiesterase and a mGluR5 blocking agent for the alleviation of a symptom of Fragile X syndrome, GRIN disorder, SynGAP1 intellectual disability or Phelan-McDermid syndrome in an individual in need thereof.

50. Use of one or more inhibitor of one or more phosphodiesterase for in the manufacture of a medicament for the alleviation of a symptom of Fragile X syndrome, GRIN disorder, SynGAP1 intellectual disability or Phelan-McDermid syndrome in an individual in need thereof.

51. Use of one or more inhibitor of one or more phosphodiesterase and a mGluR5 blocking agent in the manufacture of a medicament for the alleviation of a symptom of Fragile X syndrome, GRIN disorder, SynGAP1 intellectual disability or Phelan-McDermid syndrome in an individual in need thereof.

52. Use of one or more inhibitor of one or more phosphodiesterase in the prevention of Fragile X syndrome, GRIN disorder, SynGAP1 intellectual disability or Phelan-McDermid syndrome in an individual in need thereof.

53. Use of one or more inhibitor of one or more phosphodiesterase and a mGluR5 blocking agent in the prevention of Fragile X syndrome, GRIN disorder, SynGAP1 intellectual disability or Phelan-McDermid syndrome in an individual in need thereof.

54. Use of one or more inhibitor of one or more phosphodiesterase in the manufacture of a medicament for the prevention of Fragile X syndrome, GRIN disorder, SynGAP1 intellectual disability or Phelan-McDermid syndrome in an individual in need thereof.

55. Use of one or more inhibitor of one or more phosphodiesterase and a mGluR5 blocking agent in the manufacture of a medicament for the prevention of Fragile X syndrome, GRIN disorder, SynGAP1 intellectual disability or Phelan-McDermid syndrome in an individual in need thereof.

56. A phosphodiesterase inhibitor for use in the prevention, treatment or the alleviation of a symptom of Fragile X syndrome, GRIN disorder, SynGAP1 intellectual disability or Phelan-McDermid syndrome in an individual in need thereof.

57. A phosphodiesterase inhibitor for use in the manufacture of a medicament for the prevention, treatment or the alleviation of a symptom of Fragile X syndrome, GRIN disorder, SynGAP1 intellectual disability or Phelan-McDermid syndrome in an individual in need thereof.
